(19) United States
(12) Patent Application Publication
(10) Pub. No.: US 2024/0101970 A1
(43) Pub. Date: Mar. 28, 2024

(54) AAV-MEDIATED DELIVERY OF THERAPEUTIC ANTIBODIES TO THE INNER EAR

(71) Applicant: Akouos, Inc., Boston, MA (US)

(72) Inventors: Emmanuel John Simons, Brookline, MA (US); Robert Ng, Newton, MA (US); Michael McKenna, Boston, MA (US)

(21) Appl. No.: 18/209,041

(22) Filed: Jun. 13, 2023

Related U.S. Application Data
(63) Continuation of application No. 16/955,715, filed on Jun. 18, 2020, now abandoned.
(60) Provisional application No. 62/607,665, filed on Dec. 19, 2017.

(51) Int. Cl. C12N 7/00; A61K 9/00; A61K 48/00; A61P 27/16; C07K 16/22

(52) U.S. Cl. CPC C12N 7/00; A61K 9/0046; A61K 48/005; A61P 27/16; C07K 16/22; C12N 2750/14143

(57) ABSTRACT

Provided herein are methods that include introducing into an inner ear of a mammal a therapeutically effective amount of an adeno-associated virus (AAV) vector that includes a nucleotide sequence encoding (a) a polypeptide including an antibody heavy chain variable domain operably linked to a signal peptide and a polypeptide including an antibody light chain variable domain operably linked to a signal peptide; (b) a polypeptide including an antigen-binding antibody fragment operably linked to a signal peptide; or (c) a soluble vascular endothelial growth factor receptor operably linked to a signal peptide.

### CROSS-REFERENCE TO RELATED APPLICATIONS

This application claims priority to U.S. Provisional Patent Application Ser. No. 62/607,665, filed Dec. 19, 2017; the entire contents of which are herein incorporated by reference.

### TECHNICAL FIELD

The present disclosure relates generally to the use of nucleic acids to treat hearing loss in a human subject.

### BACKGROUND OF THE INVENTION

Sensorinerual hearing loss is hearing loss that is caused by a malfunction of the cells (e.g., hair cells) in an inner ear of a mammal. Non-limiting causes of sensorineural hearing loss include exposure to loud noise, head trauma, viral infection, automimmune inner ear disease, genetic hearing loss, aging, malformations in the inner ear, Meniere's disease, otosclerosis, and tumors.

### SUMMARY

The present invention relates to methods that include introducing into an inner ear of a mammal (e.g., a human) a therapeutically effective amount of any adeno-associated virus (AAV) vector that includes a nucleotide sequence encoding: (a) a polypeptide including an antibody heavy chain variable domain operably linked to a signal peptide and a polypeptide including an antibody light chain variable domain operably linked to a signal peptide; or (b) a polypeptide including an antigen-binding antibody fragment operably linked to a signal peptide.

Provided herein are methods for increasing the level of an antibody or an antigen-binding antibody fragment in an inner ear of a mammal in need thereof that include: introducing into the inner ear of the mammal a therapeutically effective amount of an AAV vector that includes a nucleotide sequence encoding (a) a polypeptide including an antibody heavy chain variable domain operably linked to a signal peptide and a polypeptide including an antibody light chain variable domain operably linked to a signal peptide; or (b) a polypeptide including an antigen-binding antibody fragment linked to a signal peptide; wherein the introducing results in an increase in the level of the antibody or the antigen-binding antibody fragment in the inner ear of the mammal.

In some embodiments, the antibody or the antigen-binding antibody fragment binds specifically to vascular endothelial growth factor (VEGF). In some embodiments, the antibody or antigen-binding antibody fragment decreases VEGF activity. In some embodiments of any of the methods described herein, the AAV vector further includes one or both of a promoter and a Kozak sequence that are operably linked to the sequence encoding the antibody or the antigen-binding antibody fragment.

In some embodiments, the AAV vector includes a promoter selected from the group consisting of: an inducible promoter, a constitutive promoter, and a tissue-specific promoter. In some embodiments of any of the methods described herein, the AAV vector further includes a polyadenylation signal sequence.

In some embodiments of any of the methods described herein, the mammal is a human. In some embodiments of any of the methods described herein, the mammal has been identified as having an inner ear disorder. In some embodiments of any of the methods described herein, the mammal has been diagnosed as having an inner ear disorder.

In some embodiments of any of the methods described herein, the AAV vector includes a nucleic acid sequence encoding a polypeptide including an antibody heavy chain variable domain operably linked to a signal peptide and a polypeptide including an antibody light chain variable domain operably linked to a signal peptide.

In some embodiments of any of the methods described herein, the AAV vector includes a nucleic acid sequence encoding a polypeptide including an antigen-binding antibody fragment operably linked to a signal.

Also provided herein are methods for treating an inner ear disorder in a mammal in need thereof that include: introducing into the inner ear of the mammal a therapeutically effective amount of an AAV vector that includes a nucleotide sequence encoding: (a) a polypeptide including an antibody heavy chain variable domain operably linked to a signal peptide and a polypeptide including an antibody light chain variable domain operably linked to a signal peptide; or (b) a polypeptide including an antigen-binding antibody fragment linked to a signal peptide; wherein the introducing results in the treatment of the inner ear disorder in the mammal.

In some embodiments of any of the methods described herein, the AAV vector further includes one or both of a promoter and a Kozak sequence that are operably linked to the sequence encoding the antibody or the antigen-binding antibody fragment.

In some embodiments, the AAV vector includes a promoter selected from the group consisting of: an inducible promoter, a constitutive promoter, and a tissue-specific promoter.

In some embodiments of any of the methods described herein, the AAV vector further includes a polyadenylation signal sequence.

In some embodiments of any of the methods described herein, the mammal is a human. In some embodiments of any of the methods described herein, the mammal has been identified as having an inner ear disorder. In some embodiments of any of the methods described herein, the mammal has been diagnosed as having an inner ear disorder.

In some embodiments of any of the methods described herein, the AAV vector includes a nucleic acid sequence encoding a polypeptide including an antibody heavy chain variable domain operably linked to a signal peptide and a polypeptide including an antibody light chain variable domain operably linked to a signal peptide.

In some embodiments of any of the methods described herein, the AAV vector includes a nucleic acid sequence encoding a polypeptide including an antigen-binding antibody fragment operably linked to a signal.

Also provided herein are methods of reducing VEGF activity in an inner ear of a mammal in need thereof that include: introducing into the inner ear of the mammal a therapeutically effective amount of an AAV vector that includes a nucleotide sequence encoding (a) a polypeptide including an antibody heavy chain variable domain operably linked to a signal peptide and a polypeptide including an antibody light chain variable domain operably linked to a signal peptide; or (b) a polypeptide including an antigen-binding antibody fragment linked to a signal peptide; wherein the polypeptide of (a) encodes an antibody that binds specifically to VEGF and reduces VEGF activity, the polypeptide of (b) encodes an antigen-binding antibody fragment that binds specifically to VEGF and reduces VEGF activity; wherein the introducing results in a reduction in VEGF activity in the inner ear of the mammal.

In some embodiments of any of the methods described herein, the AAV vector further includes one or both of a promoter and a Kozak sequence that are operably linked to the sequence encoding the antibody or the antigen-binding antibody fragment.

In some embodiments, the AAV vector includes a promoter selected from the group consisting of: an inducible promoter, a constitutive promoter, and a tissue-specific promoter.

In some embodiments of any of the methods described herein, the AAV vector further includes a polyadenylation signal sequence. In some embodiments of any of the methods described herein, the mammal is a human.

In some embodiments of any of the methods described herein, the mammal has been identified or diagnosed as having an acoustic neuroma. In some embodiments, the mammal has been identified or diagnosed as having a vestibular schwannoma. In some embodiments of any of the methods described herein, the mammal has been identified or diagnosed as having a neurofibromatosis type 2.

In some embodiments of any of the methods described herein, the AAV vector includes a nucleic acid sequence encoding a polypeptide including an antibody heavy chain variable domain operably linked to a signal peptide and a polypeptide including an antibody light chain variable domain operably linked to a signal peptide.

In some embodiments of any of the methods described herein, the AAV vector includes a nucleic acid sequence encoding a polypeptide including an antigen-binding antibody fragment operably linked to a signal peptide.

Also provided herein are methods of treating acoustic neuroma, vestibular schwannoma, or neurofibromatosis type 2 in an inner ear of a mammal that include: introducing into the inner ear of the mammal a therapeutically effective amount of an AAV vector that includes a nucleotide sequence encoding (a) a polypeptide including an antibody heavy chain variable domain operably linked to a signal peptide and a polypeptide including an antibody light chain variable domain operably linked to a signal peptide; or (b) a polypeptide including an antigen-binding antibody fragment linked to a signal peptide; wherein the polypeptide of (a) encodes an antibody that binds specifically to VEGF and reduces VEGF activity, the polypeptide of (b) encodes an antigen-binding antibody fragment that binds specifically to VEGF and reduces VEGF activity; wherein the introducing results in treatment of acoustic neuroma, vestibular schwannoma, or neurofibromatosis type II, respectively, in the inner ear of the mammal.

In some embodiments of any of the methods described herein, the AAV vector further includes one or both of a promoter and a Kozak sequence that are operably linked to the sequence encoding the antibody or the antigen-binding antibody fragment.

In some embodiments of any of the methods described herein, the AAV vector includes a promoter selected from the group consisting of: an inducible promoter, a constitutive promoter, and a tissue-specific promoter.

In some embodiments of any of the methods described herein, the AAV vector further includes a polyadenylation signal sequence.

In some embodiments of any of the methods described herein, the mammal is a human. In some embodiments of any of the methods described herein, the mammal has been identified or diagnosed as having an acoustic neuroma. In some embodiments of any of the methods described herein, the mammal has been identified or diagnosed as having a vestibular schwannoma. In some embodiments of any of the methods described herein, the mammal has been identified or diagnosed as having neurofibromatosis type 2.

In some embodiments of any of the methods described herein, the AAV vector includes a nucleic acid sequence encoding a polypeptide including an antibody heavy chain variable domain operably linked to a signal peptide and a polypeptide including an antibody light chain variable domain operably linked to a signal peptide.

In some embodiments of any of the methods described herein, the AAV vector includes a nucleic acid sequence encoding a polypeptide including an antigen-binding antibody fragment operably linked to a signal peptide.

In some embodiments of any of the methods described herein, the antibody includes a Fc region that includes one or more amino acid substitutions that decreases the half-life of the antibody in a mammal as compared to a control antibody; or the antigen-binding antibody fragment thereof has a decreased in vivo half-life as compared to a control antigen-binding antibody fragment.

Also provided herein are methods that include introducing into an inner ear of a mammal a therapeutically effective amount of an adeno-associated virus (AAV) vector that includes a nucleotide sequence encoding a soluble vascular endothelial growth factor (VEGF) receptor operably linked to a signal peptide.

Also provided herein are methods for increasing the level of a soluble vascular endothelial growth factor (VEGF) receptor in an inner ear of a mammal in need thereof that include: introducing into the inner ear of the mammal a therapeutically effective amount of an AAV vector that includes a nucleotide sequence encoding a soluble VEGF receptor operably linked to a signal peptide; where the introducing results in an increase in the level of the soluble VEGF receptor in the inner ear of the mammal.

In some embodiments of any of the methods described herein, the soluble VEGF receptor includes a portion of an extracellular region of VEGF receptor-1 (VEGFR-1). In some embodiments of any of the methods described herein, the portion of the extracellular region of VEGFR-1 includes a contiguous sequence from wildtype human VEGFR-1. In some embodiments of any of the methods described herein, the portion of the extracellular region of VEGFR-1 includes one or more immunoglobulin-like domains in the extracellular region from wildtype human VEGFR-1. In some embodiments of any of the methods described herein, the portion of the extracellular region of VEGFR-1 includes a sequence that is at least 90% identical to a contiguous sequence from wildtype human VEGFR-1.

In some embodiments of any of the methods described herein, the soluble VEGF receptor includes a portion of an extracellular region of VEGF receptor-2 (VEGFR-2). In some embodiments of any of the methods described herein, the portion of the extracellular region of VEGFR-2 includes a contiguous sequence from wildtype human VEGFR-2. In some embodiments of any of the methods described herein, the portion of the extracellular region of VEGFR-2 includes one or more immunoglobulin-like domains in the extracellular region from wildtype human VEGFR-2. In some embodiments of any of the methods described herein, the portion of the extracellular region of VEGFR-2 includes a sequence that is at least 90% identical to a contiguous sequence from wildtype human VEGFR-2.

In some embodiments of any of the methods described herein, the soluble VEGF receptor includes a portion of an extracellular region of VEGFR-1 and a portion of an extracellular region of VEGFR-2. In some embodiments of any of the methods described herein, the portion of the extracellular region of VEGFR-1 includes one or more immunoglobulin-like domains in the extracellular region from wildtype human VEGFR-1; and the portion of the extracellular region of VEGFR-2 includes one or more immunoglobulin-like domains in the extracellular region from wildtype human VEGFR-2. In some embodiments of any of the methods described herein, the soluble VEGF receptor is aflibercept.

In some embodiments of any of the methods described herein, the soluble VEGF receptor includes a portion of an extracellular region of VEGF receptor-3 (VEGFR-3). In some embodiments of any of the methods described herein, the portion of the extracellular region of VEGFR-3 includes a contiguous sequence from wildtype human VEGFR-3. In some embodiments of any of the methods described herein, the portion of the extracellular region of VEGFR-3 includes one or more immunoglobulin-like domains in the extracellular region from wildtype human VEGFR-3. In some embodiments of any of the methods described herein, the portion of the extracellular region of VEGFR-3 includes a sequence that is at least 90% identical to a contiguous sequence from wildtype human VEGFR-3. In some embodiments of any of the methods described herein, the soluble VEGF receptor comprises a Fc domain. In some embodiments of any of the methods described herein, the Fc domain is an IgG1 Fc domain. In some embodiments of any of the methods described herein, the IgG1 Fc domain is a human wildtype IgG1 Fc domain.

In some embodiments of any of the methods described herein, the soluble VEGF receptor decreases the ability of a VEGF to bind to one or more of VEGFR-1, VEGFR-2, and VEGFR-3.

In some embodiments of any of the methods described herein, the AAV vector further includes one or both of a promoter and a Kozak sequence that are operably linked to the sequence encoding the soluble VEGF receptor. In some embodiments of any of the methods described herein, the AAV vector includes a promoter selected from the group of: an inducible promoter, a constitutive promoter, and a tissue-specific promoter. In some embodiments of any of the methods described herein, the AAV vector further includes a polyadenylation signal sequence.

In some embodiments of any of the methods described herein, the mammal is a human. In some embodiments of any of the methods described herein, the mammal has been identified as having an inner ear disorder. In some embodiments of any of the methods described herein, the mammal has been diagnosed as having an inner ear disorder.

Also provided herein are methods for treating an inner ear disorder in a mammal in need thereof that include: introducing into the inner ear of the mammal a therapeutically effective amount of an AAV vector that includes a nucleotide sequence encoding a soluble vascular endothelial growth factor (VEGF) receptor operably linked to a signal peptide; where the introducing results in the treatment of the inner ear disorder in the mammal.

In some embodiments of any of the methods described herein, the AAV vector further includes one or both of a promoter and a Kozak sequence that are operably linked to the sequence encoding the soluble VEGF receptor. In some embodiments of any of the methods described herein, the AAV vector includes a promoter selected from the group of: an inducible promoter, a constitutive promoter, and a tissue-specific promoter. In some embodiments of any of the methods described herein, the AAV vector further includes a polyadenylation signal sequence.

In some embodiments of any of the methods described herein, the mammal is a human. In some embodiments of any of the methods described herein, the mammal has been identified as having an inner ear disorder. In some embodiments of any of the methods described herein, the mammal has been diagnosed as having an inner ear disorder.

Also provided herein are methods of reducing a VEGF activity in an inner ear of a mammal in need thereof that include: introducing into the inner ear of the mammal a therapeutically effective amount of an AAV vector that includes a nucleotide sequence encoding a soluble vascular endothelial growth factor (VEGF) receptor operably linked to a signal peptide; where the introducing results in a reduction in the VEGF activity in the inner ear of the mammal.

In some embodiments of any of the methods described herein, the AAV vector further includes one or both of a promoter and a Kozak sequence that are operably linked to the sequence encoding the soluble VEGF receptor. In some embodiments of any of the methods described herein, the AAV vector includes a promoter selected from the group of: an inducible promoter, a constitutive promoter, and a tissue-specific promoter. In some embodiments of any of the methods described herein, the AAV vector further includes a polyadenylation signal sequence.

In some embodiments of any of the methods described herein, the mammal is a human. In some embodiments of any of the methods described herein, the mammal has been identified or diagnosed as having an acoustic neuroma. In some embodiments of any of the methods described herein, the mammal has been identified or diagnosed as having a vestibular schwannoma. In some embodiments of any of the methods described herein, the mammal has been identified or diagnosed as having a neurofibromatosis type 2.

Also provided herein are methods of treating acoustic neuroma, vestibular schwannoma, or neurofibromatosis type 2 in an inner ear of a mammal that include: introducing into the inner ear of the mammal a therapeutically effective amount of an AAV vector that includes a nucleotide sequence encoding a nucleotide sequence encoding a soluble vascular endothelial growth factor (VEGF) receptor operably linked to a signal peptide; where the introducing results in treatment of acoustic neuroma, vestibular schwannoma, or neurofibromatosis type II, respectively, in the inner ear of the mammal.

In some embodiments of any of the methods described herein, the AAV vector further includes one or both of a promoter and a Kozak sequence that are operably linked to the sequence encoding the soluble VEGF receptor. In some embodiments of any of the methods described herein, the AAV vector includes a promoter selected from the group of: an inducible promoter, a constitutive promoter, and a tissue-specific promoter. In some embodiments of any of the methods described herein, the AAV vector further includes a polyadenylation signal sequence.

In some embodiments of any of the methods described herein, the mammal is a human. In some embodiments of any of the methods described herein, the mammal has been identified or diagnosed as having an acoustic neuroma. In some embodiments of any of the methods described herein, the mammal has been identified or diagnosed as having a vestibular schwannoma. In some embodiments of any of the methods described herein, the mammal has been identified or diagnosed as having neurofibromatosis type 2.

In some embodiments of any of the methods described herein, the soluble VEGF receptor includes a portion of an extracellular region of VEGF receptor-1 (VEGFR-1). In some embodiments of any of the methods described herein, the portion of the extracellular region of VEGFR-1 includes a contiguous sequence from wildtype human VEGFR-1. In some embodiments of any of the methods described herein, the portion of the extracellular region of VEGFR-1 includes one or more immunoglobulin-like domains in the extracellular region from wildtype human VEGFR-1. In some embodiments of any of the methods described herein, the portion of the extracellular region of VEGFR-1 includes a sequence that is at least 90% identical to a contiguous sequence from wildtype human VEGFR-1.

In some embodiments of any of the methods described herein, the soluble VEGF receptor includes a portion of an extracellular region of VEGF receptor-2 (VEGFR-2). In some embodiments of any of the methods described herein, the portion of the extracellular region of VEGFR-2 includes a contiguous sequence from wildtype human VEGFR-2. In some embodiments of any of the methods described herein, the portion of the extracellular region of VEGFR-2 includes one or more immunoglobulin-like domains in the extracellular region from wildtype human VEGFR-2. In some embodiments of any of the methods described herein, the portion of the extracellular region of VEGFR-2 includes a sequence that is at least 90% identical to a contiguous sequence from wildtype human VEGFR-2.

In some embodiments of any of the methods described herein, the soluble VEGF receptor includes a portion of an extracellular region of VEGFR-1 and a portion of an extracellular region of VEGFR-2. In some embodiments of any of the methods described herein, the portion of the extracellular region of VEGFR-1 includes one or more immunoglobulin-like domains in the extracellular region from wildtype human VEGFR-1; and the portion of the extracellular region of VEGFR-2 includes one or more immunoglobulin-like domains in the extracellular region from wildtype human VEGFR-2. In some embodiments of any of the methods described herein, the soluble VEGF receptor is aflibercept.

In some embodiments of any of the methods described herein, the soluble VEGF receptor includes a portion of an extracellular region of VEGF receptor-3 (VEGFR-3). In some embodiments of any of the methods described herein, the portion of the extracellular region of VEGFR-3 includes a contiguous sequence from wildtype human VEGFR-3. In some embodiments of any of the methods described herein, the portion of the extracellular region of VEGFR-3 includes one or more immunoglobulin-like domains in the extracellular region from wildtype human VEGFR-3. In some embodiments of any of the methods described herein, the portion of the extracellular region of VEGFR-3 includes a sequence that is at least 90% identical to a contiguous sequence from wildtype human VEGFR-3.

In some embodiments of any of the methods described herein, the soluble VEGF receptor comprises a Fc domain. In some embodiments of any of the methods described herein, the Fc domain is an IgG1 Fc domain. In some embodiments of any of the methods described herein, the IgG1 Fc domain is a human wildtype IgG1 Fc domain.

In some embodiments of any of the methods described herein, the soluble VEGF receptor decreases the ability of a VEGF to bind to one or more of VEGFR-1, VEGFR-2, and VEGFR-3. In some embodiments of any of the methods described herein, the AAV vector further includes a secretion sequence.

Unless otherwise specified, a “nucleotide sequence encoding an amino acid sequence” includes all nucleotide sequences that are degenerate versions of each other and thus encode the same amino acid sequence.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “transfected,” “transformed,” or “transduced” refers to a process by which exogenous nucleic acid is transferred or introduced into a cell. A “transfected,” “transformed,” or “transduced” mammalian cell is one that has been transfected, transformed, or transduced with exogenous nucleic acid.

The term “expression” refers to the transcription and/or translation of a particular nucleotide sequence encoding a protein.

The term “transient expression” refers to the expression of a non-integrated coding sequence for a short period of time (e.g., hours or days). The coding sequence that is transiently expressed in a cell (e.g., a mammalian cell) is lost upon multiple rounds of cell division.

The term “subject” is intended to include any mammal. In some embodiments, the subject is a rodent (e.g., a rat or mouse), a rabbit, a sheep, a goat, a pig, a dog, a cat, a non-human primate, or a human. In some embodiments, the subject has or is at risk of developing non-syndromic deafness. In some embodiments, the subject has been previously identified as having an inner ear disorder. In some embodiments, the subject has previously been diagnosed as having an inner ear disorder. In some embodiments, the subject has been identified as having drug-induced hearing loss. In some embodiments, the subject is an infant (e.g., a human infant).

A treatment is “therapeutically effective” when it results in a reduction in one or more of the number, severity, and frequency of one or more symptoms of a disease (e.g., non-symptomatic sensorineural hearing loss) in a subject (e.g., a human).

The term “nucleic acid” or “polynucleotide” refers to deoxyribonucleic acid (DNA) or ribonucleic acid (RNA), or a combination thereof, in either single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

The term “signal peptide” refers to a sequence present on the N-terminus of a nascent secreted protein but is absent in the naturally-occurring mature protein. A “signal peptide” is cleaved by a protease (e.g., a signal peptidase) after the signal peptide is translated. Signal peptides are known in the art. Non-limiting examples of signal peptides include: MEFFKKTALAALVMGFSGAALA (SEQ ID NO: 9) and MKYLLPTAAAGLLLLAAQPAMA (SEQ ID NO: 10).

The term “inner ear disorder” refers to a disorder caused by malfunction of the cells (e.g., hair cells, supporting cells, spiral ganglion neurons, macrophages, or schwann cells) in or around the inner ear of a mammal. Non-limiting examples of inner ear disorders include, e.g., sensorineural hearing loss (SNHL), noise-induced hearing loss, drug-induced hearing loss, age-related hearing loss, acoustic neuroma, neurofibromatosis type 2, auditory neuropathy, noise-induced cochlear synaptopathy without hair cell loss, age-related cochlear synaptopathy, acquired sensorineural hearing loss, and vestibular schwannoma. See, e.g., Kujawa et al., Hear Res 330(0 0): 191-199, 2015; and Suzuki et al., Scientific Reports 6: 24907. Non-limiting examples of inner ear disorders are described herein and additional examples of inner ear disorders are known in the art.

The term “antibody” means a complex of two or more single polypeptide chains that interact to form at least one antigen-binding domain. Non-limiting examples of an antibody include monoclonal antibodies (for example, full length or intact monoclonal antibodies), polyclonal antibodies, multivalent antibodies, multispecific antibodies (e.g., bispecific, trispecific, etc. antibodies so long as they exhibit the desired biological activity). An antibody can be human, humanized, and/or affinity-matured.

The term “antigen-binding antibody fragment” is a single polypeptide that includes all the amino acids that make up at least one antigen-binding domain (e.g., an scFv).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al, Proc. Natl. Acad. Sci. USA 81:6851-6855 (1984)).

An “antigen-binding domain” is one or more protein domain(s) (e.g., formed from amino acids from a single polypeptide or formed from amino acids from two or more polypeptides (e.g., the same or different polypeptides) that is capable of specifically binding to one or more different antigens. In some examples, an antigen-binding domain can bind to an antigen or epitope with specificity and affinity similar to that of naturally-occurring antibodies. In some embodiments, an antigen-binding domain can include an alternative scaffold. Non-limiting examples of antigen-binding domains are described herein. Additional examples of antigen-binding domains are known in the art. In some examples, an antigen-binding domain can bind to a single antigen.

“Affinity” refers to the strength of the sum total of non-covalent interactions between an antigen-binding site and its binding partner (e.g., an antigen or epitope). Unless indicated otherwise, as used herein, “affinity” refers to intrinsic binding affinity, which reflects a 1:1 interaction between members of an antigen-binding domain and an antigen or epitope. The affinity of a molecule X for its partner Y can be represented by the dissociation equilibrium constant (K_{D}). Affinity can be measured by common methods known in the art, including those described herein. Affinity can be determined, for example, using surface plasmon resonance (SPR) technology (e.g., BIACORE®) or biolayer interferometry (e.g., FORTEBIO®). Additional methods for determining the affinity between an antigen-binding domain and its corresponding antigen or epitope are known in the art.

The phrase “half-life” refers to the half-life of an antibody, an antigen-binding antibody fragment thereof, or a soluble VEGF receptor in circulation (e.g., blood) of a mammal (e.g., any of the mammals described herein) and is represented by the time required for 50% of an antibody, an antigen-binding antibody fragment thereof, or soluble VEGF receptor to be cleared from the circulation. In some embodiments, an alteration in half-life (e.g., a decrease in half-life of an antibody, an antigen-binding antibody fragment thereof, or soluble VEGF receptor) is determined by comparing the half-life of an antibody, an antigen-binding antibody fragment, or a soluble VEGF receptor in a subject to the half-life of a control antibody, control antigen-binding antibody fragment, or control soluble VEGF receptor in a similar mammal.

In some embodiments, the half-life of an antibody, antigen-binding antibody fragment thereof, or soluble VEGF receptor in a mammal is determined by measuring the level of the antibody, antigen-binding antibody fragment thereof, or soluble VEGF receptor in samples obtained from a subject (e.g., a blood sample) at different time points following systemic administration (e.g., intravenous) administration of any of the AAV vectors described herein. In some embodiments, the level of the antibody, antigen-binding antibody fragment thereof, or soluble VEGF receptor present in samples obtained from a mammal is determined using enzyme-linked immunosorbent assay (ELISA) or another assay known to the art, and the determined level of the antibody, antigen-binding antibody fragment thereof, or soluble VEGF receptor present in the samples is plotted as a function of time using a software program (e.g., GraphPad Prism).

The term “VEGF activity” refers to one or more known activities of a VEGF protein. For example, one activity of a VEGF protein is the ability to bind to one or more VEGF receptors. In another example, one activity of a VEGF protein is the ability of a VEGF to trigger downstream signal transduction pathway(s) in a mammalian cell expressing a VEGF receptor. Methods for detecting one or more activities of VEGF are known in the art.

The term “soluble VEGF receptor” refers to a polypeptide that includes a portion of an extracellular region of one or more mammalian VEGF receptor(s) (e.g., VEGFR-1, VEGFR-2, and VEGFR-3) operably linked to a signal peptide, where the soluble VEGF receptor is capable of specifically binding to one or more mammalian VEGF proteins (e.g., one or more of VEGF-A, VEGF-B, VEGF-C, and VEGF-D). In some examples, a soluble VEGF receptor includes a portion of an extracellular region of VEGFR-1 (e.g., a contiguous sequence from wildtype human VEGFR-1 (e.g., one or more immunoglobulin-like domains in the extracellular region from wildtype human VEGFR-1) or a sequence that is at least 90% identical to a contiguous sequence from wildtype human VEGFR-1). In some examples, a soluble VEGF receptor includes a portion of an extracellular region of VEGFR-2 (e.g., a contiguous sequence from wildtype human VEGFR-2 (e.g., one or more immunoglobulin-like domains in the extracellular region from wildtype human VEGFR-2) or a sequence that is at least 90% identical to a contiguous sequence from wildtype human VEGFR-2). In some examples, a soluble VEGF receptor includes a portion of an extracellular region of VEGFR-1 and a portion of an extracellular region of VEGFR-2 (e.g., one or more immunoglobulin-like domains in the extracellular region from wildtype human VEGFR-1 and one or more immunoglobulin-like domains in the extracellular region from wildtype human VEGFR-2) (e.g., aflibercept). In some examples, a soluble VEGF receptor includes a portion of an extracellular region of VEGFR-3 (e.g., a contiguous sequence from wildtype human VEGFR-3 (e.g., one or more immunoglobulin-like domains in the extracellular region from wildtype human VEGFR-3) or a sequence that is at least 90% identical to a contiguous sequence from wildtype human VEGFR-3).

In some examples, a soluble VEGF receptor can further include a stabilizing domain (e.g., a Fc domain, such as an IgG1 Fc domain (e.g., a human wildtype IgG1 Fc domain). In some examples, the soluble VEGF receptor decreases the ability of a VEGF to bind to one or more (e.g., two or three) of VEGFR-1, VEGFR-2, and VEGFR-3.Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF DRAWINGS

FIG. 1A is an exemplary AAV vector of 4474 bp that includes a sequence encoding bevacizumab (Avastin®).

FIG. 1B is an exemplary AAV vector of 3814 bp that includes a sequence encoding ranibizumab (Lucentis®).

FIG. 1C is an exemplary AAV vector of 4573 bp that includes a sequence encoding ranibizumab and green fluorescent protein (GFP).

FIG. 1D is an exemplary AAV vector of 3631 bp that includes a sequence encoding aflibercept (Eylea®).

FIG. 2 is a Western blot showing HEK cell expression of different anti-VEGF antibodies or antigen-binding antibody fragments, or soluble VEGF receptors using exemplary AAV vectors described herein. Lane 1: pre-stained PageRuler™ protein ladder. Lane 2: untransfected/ negative control. Lane 3: tranfection with the AAV vector shown in FIG. 1A. Lane 4: transfection with the AAV vector shown in FIG. 1C. Lane 5: transfection with the AAV vector shown in FIG. 1B. Lane 6: transfection with the AAV vector shown in FIG. 1A with an multiplicity of infection (MOI) of 7.5×10⁴. Lane 7: transfection with the AAV vector shown in FIG. 1A with an MOI of 2.2×10⁵. Lane 8: transfection with the AAV vector shown in FIG. 1A with an MOI of 5.5×10⁵. Lane 9: prestained PageRuler™ protein ladder. Lane 10: untransfected/negative control. Lane 11: tranfection with the AAV vector shown in FIG. 1A. Lane 12: transfection with the AAV vector shown in FIG. 1C. Lane 13: transfection with the AAV vector shown in FIG. 1B. Lane 14: transfection with the AAV vector shown in FIG. 1A with an multiplicity of infection (MOI) of 7.5×10⁴. Lane 15: transfection with the AAV vector shown in FIG. 1A with an MOI of 2.2×10⁵. Lane 16: transfection with the AAV vector shown in FIG. 1A with an MOI of 5.5×10⁵. Lanes 2-8 contain reduced proteins. Lanes 10-16 contain non-reduced proteins.

FIG. 3A is a graph showing the affinity of a control mouse anti-human VEGF monoclonal antibody (anti-hVEGF MmAb) in a buffer using recombinant human VEGF as the binding agent, as measured by Octet® HTX biosensor instrument using the Octet® analysis software, Data Analysis HT10.0.

FIG. 3B is a graph showing the affinity of a control anti-hVEGF MmAb in conditioned media (CM) samples using recombinant human VEGF as the binding agent, as measured by Octet® HTX biosensor instrument using the Octet® analysis software, Data Analysis HT10.0. *: anti-hVEGF MmAb was prepared in CM at 100 μg/mL, then diluted to a final concentration of 10 μg/mL in 1×kinetics buffer.

FIG. 4A is a graph showing the affinity of conditioned medium using recombinant human VEGF as the binding agent, as measured by Octet® HTX biosensor instrument using the Octet® analysis software, Data Analysis HT10.0.

FIG. 4B is a graph showing the affinity of culture medium from HEK cells transfected with the AAV vector shown in FIG. 1A using recombinant human VEGF as the binding agent, using by Octet® HTX biosensor instrument using the Octet® analysis software, Data Analysis HT10.0.

FIG. 4C is a table showing the equilibrium dissociation constant (K_{D}) determined from the data shown in FIGS. 3A, 3B, 4A, and 4B (going from the top to the bottom of the table).

### DETAILED DESCRIPTION

Provided herein are methods that include introducing into an inner ear of a mammal a therapeutically effective amount of an adeno-associated virus (AAV) vector that includes a nucleotide sequence encoding: (a) a polypeptide including an antibody heavy chain variable domain operably linked to a signal peptide and a polypeptide including an antibody light chain variable domain operably linked to a signal peptide; or (b) a polypeptide including an antigen-binding antibody fragment (e.g., a Fab or a scFv)operably linked to a signal peptide.

Also provided herein are methods for increasing the level of an antibody or an antigen-binding antibody fragment in an inner ear of a mammal in need thereof, that include: introducing into the inner ear of the mammal a therapeutically effective amount of an AAV vector that includes a nucleotide sequence encoding: (a) a polypeptide including an antibody heavy chain variable domain operably linked to a signal peptide and a polypeptide including an antibody light chain variable domain operably linked to a signal peptide; or (b) a polypeptide including an antigen-binding antibody fragment (e.g., a Fab or a scFv) operably linked to a signal peptide; wherein the introducing results in an increase in the level of the antibody or the antigen-binding antibody fragment in the inner ear of the mammal.

Also provided are methods for treating an inner ear disorder in a mammal in need thereof that include introducing into the inner ear of the mammal a therapeutically effective amount of an AAV vector that includes a nucleotide sequence encoding: (a) a polypeptide including an antibody heavy chain variable domain operably linked to a signal peptide and a polypeptide comprising an antibody light chain variable domain operably linked to a signal peptide; or (b) a polypeptide comprising an antigen-binding antibody fragment linked to a signal peptide; where the introducing results in the treatment of the inner ear disorder in the mammal.

Also provided herein are methods of reducing VEGF activity in an inner ear of a mammal in need thereof that include: introducing into the inner ear of the mammal a therapeutically effective amount of an AAV vector that includes a nucleotide sequence encoding (a) a polypeptide including an antibody heavy chain variable domain operably linked to a signal peptide and a polypeptide including an antibody light chain variable domain operably linked to a signal peptide; or (b) a polypeptide including an antigen-binding antibody fragment (e.g., a Fab or a scFv) operably linked to a signal peptide; wherein the polypeptide of (a) encodes an antibody that binds specifically to VEGF and reduces VEGF activity, the polypeptide of (b) encodes an antigen-binding antibody fragment that binds specifically to VEGF and reduces VEGF activity; and wherein the introducing results in a reduction in VEGF activity in the inner ear of the mammal.

Also provided herein are methods of treating acoustic neuroma, vestibular schwannoma, or neurofibromatosis type II in an inner ear of a mammal that include: introducing into the inner ear of the mammal a therapeutically effective amount of an AAV vector that includes a nucleotide sequence encoding (a) a polypeptide including an antibody heavy chain variable domain operably linked to a signal peptide and a polypeptide including an antibody light chain variable domain operably linked to a signal peptide; or (b) a polypeptide including an antigen-binding antibody fragment (e.g., a Fab or a scFv) operably linked to a signal peptide; wherein the polypeptide of (a) encodes an antibody that binds specifically to VEGF and reduces VEGF activity, the polypeptide of (b) encodes an antigen-binding antibody fragment that binds specifically to VEGF and reduces VEGF activity; and wherein the introducing results in treatment of acoustic neuroma or vestibular schwannoma in the inner ear of the mammal.

Also provided herein are methods that include introducing into an inner ear of a mammal a therapeutically effective amount of an adeno-associated virus (AAV) vector that include a nucleotide sequence encoding a soluble vascular endothelial growth factor (VEGF) receptor operably linked to a signal peptide.

Also provided herein are methods for increasing the level of a soluble vascular endothelial growth factor (VEGF) receptor in an inner ear of a mammal in need thereof that include introducing into the inner ear of the mammal a therapeutically effective amount of an AAV vector that includes a nucleotide sequence encoding a soluble VEGF receptor operably linked to a signal peptide; where the introducing results in an increase in the level of the soluble VEGF receptor in the inner ear of the mammal.

Also provided herein are methods for treating an inner ear disorder in a mammal in need thereof that include introducing into the inner ear of the mammal a therapeutically effective amount of an AAV vector that includes a nucleotide sequence encoding a soluble vascular endothelial growth factor (VEGF) receptor operably linked to a signal peptide; where the introducing results in the treatment of the inner ear disorder in the mammal.

Also provided herein are methods of reducing a VEGF activity in an inner ear of a mammal in need thereof that include introducing into the inner ear of the mammal a therapeutically effective amount of an AAV vector that includes a nucleotide sequence encoding a soluble vascular endothelial growth factor (VEGF) receptor operably linked to a signal peptide; where the introducing results in a reduction in the VEGF activity in the inner ear of the mammal.

Also provided herein are methods of treating acoustic neuroma, vestibular schwannoma, or neurofibromatosis type 2 in an inner ear of a mammal that include introducing into the inner ear of the mammal a therapeutically effective amount of an AAV vector that includes a nucleotide sequence encoding a nucleotide sequence encoding a soluble vascular endothelial growth factor (VEGF) receptor operably linked to a signal peptide; where the introducing results in treatment of acoustic neuroma, vestibular schwannoma, or neurofibromatosis type II, respectively, in the inner ear of the mammal.

Also provided are kits that include any of the AAV vectors described herein.

Additional non-limiting aspects of the compositions, kits, and methods are described herein and can be used in any combination without limitation.

### Antibodies and Antigen-Binding Antibody Fragments

In some embodiments, the antibody can be a humanized antibody, a chimeric antibody, or a multivalent antibody. In some embodiments, an antibody or an antigen-binding antibody fragment can be a scFv-Fc, a V_{H}H domain, a V_{NAR} domain, a (scFv)₂, a minibody, or a BiTE. In some embodiments, an antibody or an antigen-binding antibody fragment can be a DVD-Ig, and a dual-affinity re-targeting antibody (DART), a triomab, kih IgG with a common LC, a crossmab, an ortho-Fab IgG, a 2-in-1-IgG, IgG-ScFv, scFv₂-Fc, a bi-nanobody, tanden antibody, a DART-Fc, a scFv-HAS-scFv, DNL-Fab3, DAF (two-in-one or four-in-one), DutaMab, DT-IgG, knobs-in-holes common LC, knobs-in-holes assembly, charge pair antibody, Fab-arm exchange antibody, SEEDbody, Triomab, LUZ-Y, Fcab, kλ-body, orthogonal Fab, DVD-IgG, IgG(H)-scFv, scFv-(H)IgG, IgG(L)-scFv, scFv-(L)-IgG, IgG (L,H)-Fc, IgG(H)-V, V(H)-IgG, IgG(L)-V, V(L)-IgG, KIH IgG-scFab, 2scFv-IgG, IgG-2scFv, scFv4-Ig, Zybody, DVI-IgG, nanobody, nanobody-HSA, a diabody, a TandAb, scDiabody, scDiabody-CH3, Diabody-CH3, Triple Body, miniantibody, minibody, TriBi minibody, scFv-CH3 KIH, Fab-scFv, scFv-CH-CL-scFv, F(ab′)₂-scFV₂, scFv-KIH, Fab-scFv-Fc, tetravalent HCAb, scDiabody-Fc, diabody-Fc, tandem scFv-Fc, intrabody, dock and lock bispecific antibody, ImmTAC, HSAbody, scDiabody-HAS, tandem scFv, IgG-IgG, Cov-X-Body, and seFv1-PEG-seFv2.

Additional examples of an antibody or an antigen-binding antibody fragment include an Fv fragment, a Fab fragment, a F(ab′)₂ fragment, and a Fab′ fragment. Additional examples of an antibody or an antigen-binding antibody fragment include an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

Any of the antibodies or antigen-binding antibody fragments described herein can bind specifically to VEGF.

A V_{H}H domain is a single monomeric variable antibody domain that can be found in camelids. A V_{NAR} domain is a single monomeric variable antibody domain that can be found in cartilaginous fish. Non-limiting aspects of V_{H}H domains and V_{NAR} domains are described in, e.g., Cromie et al., Curr. Top. Med. Chem. 15:2543- 2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr. Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

A “Fv” fragment includes a non-covalently-linked dimer of one heavy chain variable domain and one light chain variable domain.

A “Fab” fragment includes, the constant domain of the light chain and the first constant domain (C_{H1}) of the heavy chain, in addition to the heavy and light chain variable domains of the Fv fragment.

A “F(ab′)₂” fragment includes two Fab fragments joined, near the hinge region, by disulfide bonds.

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

DARTs are described in, e.g., Garber, Nature Reviews Drug Discovery 13:799-801, 2014.

Additional aspects of antibodies and antigen-binding antibody fragments are known in the art.

In some embodiments, any of the antibodies or antigen-binding antibody fragments described herein has a dissociation constant (K_{D}) of less than 1×10⁻⁵ M (e.g., less than 0.5×10⁻⁵ M, less than 1×10⁻⁶ M, less than 0.5×10⁻⁶ M, less than 1×10⁻⁷ M, less than 0.5×10⁻⁷ M, less than 1×10⁻⁸ M, less than 0.5×10⁻⁸ M, less than 1×10⁻⁹ M, less than 0.5×10⁻⁹ M, less than 1×10⁻¹⁰ M, less than 0.5×10⁻¹⁰ M, less than 1×10⁻¹¹ M, less than 0.5×10⁻¹¹ M, or less than 1×10⁻¹² M), e.g., as measured in phosphate buffered saline using surface plasmon resonance (SPR) for a VEGF protein (e.g., any of the VEGF proteins described herein, e.g., one or more of mature human VEGF-A, mature human VEGF-B, mature human VEGF-C, and mature human VEGF-D).

In some embodiments, any of the antibodies or antigen-binding antibody fragments described herein has a K_{D} of about 1×10⁻¹² M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, about 0.5×10⁻¹⁰ M, about 1×10⁻¹¹ M, or about 0.5×10⁻¹¹ M (inclusive); about 0.5×10⁻¹¹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, about 0.5×10¹⁰ M, or about 1×10⁻¹¹ M (inclusive); about 1×10⁻¹¹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, or about 0.5×10⁻¹⁰ M (inclusive); about 0.5×10⁻¹⁰ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, or about 1×10⁻¹⁰ M (inclusive); about 1×10⁻¹⁰ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, or about 0.5×10⁻⁹ M (inclusive); about 0.5×10⁻⁹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, or about 1×10⁻⁹ M (inclusive); about 1×10⁻⁹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, or about 0.5×10⁻⁸ M (inclusive); about 0.5×10⁻⁸ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, or about 1×10⁻⁸ M (inclusive); about 1×10⁻⁸ M to about 1×10⁻⁵ M, about 0.5×10⁻⁵ M, about 1×10⁻⁶ M, about 0.5×10⁻⁶ M, about 1×10⁻⁷ M, or about 0.5×10⁻⁷ M (inclusive); about 0.5×10⁻⁷ M to about 1×10⁻⁵ M, about 0.5×10⁻⁵ M, about 1×10⁻⁶ M, about 0.5×10⁻⁶ M, or about 1×10⁻⁷ M (inclusive); about 1×10⁻⁷ M to about 1×10⁻⁵ M, about 0.5×10⁻⁵ M, about 1×10⁻⁶ M, or about 0.5×10⁻⁶ M (inclusive); about 0.5×10⁻⁶ M to about 1×10⁻⁵ M, about 0.5×10⁻⁵ M, or about 1×10⁻⁶ M (inclusive); about 1×10⁻⁶ M to about 1×10⁻⁵ M or about 0.5×10⁻⁵ M (inclusive); or about 0.5×10⁻⁵ M to about 1×10⁻⁵ M (inclusive), e.g., as measured in phosphate buffered saline using surface plasmon resonance (SPR), for a VEGF protein (e.g., any of the VEGF proteins described herein, e.g., one or more of mature human VEGF-A, mature human VEGF-B, mature human VEGF-C, and mature human VEGF-D).

A variety of different methods known in the art can be used to determine the K_{D} values of any of the antibodies or antigen-binding antibody fragments described herein (e.g., an electrophoretic mobility shift assay, a filter binding assay, surface plasmon resonance, and a biomolecular binding kinetics assay, etc.).

In some embodiments of any of the antibodies and/or antigen-binding antibody fragments described herein, the half-life of the antibody and/or the antigen-binding antibody fragment in a subject (e.g., a human) is decreased about 0.5-fold to about 4-fold (e.g., about 0.5-fold to about 3.5-fold, about 0.5-fold to about 3-fold, about 0.5-fold to about 2.5-fold, about 0.5-fold to about 2-fold, about 0.5-fold to about 1.5-fold, about 0.5-fold to about 1-fold, about 1-fold to about 4-fold, about 1-fold to about 3.5-fold, about 1-fold to about 3-fold, about 1-fold to about 2.5-fold, about 1-fold to about 2-fold, about 1.5-fold to about 4-fold, about 1.5-fold to about 3.5-fold, about 1.5-fold to about 3-fold, about 1.5-fold to about 2.5-fold, about 1.5-fold to about 2-fold, about 2-fold to about 4-fold, about 2-fold to about 3.5-fold, about 2-fold to about 3-fold, about 2-fold to about 2.5-fold, about 2.5-fold to about 4-fold, about 2.5-fold to about 3.5-fold, about 2.5-fold to about 3-fold, about 3-fold to about 4-fold, about 3-fold to about 3.5-fold, or about 3.5-fold to about 4-fold) as compared to the half-life of a control antibody and/or a control antigen-binding antibody fragment (e.g., any of the control antibodies and control antigen-binding antibody fragments described herein) in a similar subject. See, e.g., Leabman et al., MAbs. 5(6): 896-903, 2013. In some embodiments, an antibody or antigen-binding antibody fragment described herein has one or more amino acid substitutions in the Fc region that decrease its half-life in a mammal, and a control antibody lacks at least one (e.g., lacks all) of these one or more amino acid substitutions in the Fc region.

### VEGF

The VEGF gene encodes vascular endothelial growth factor (VEGF), formerly known as fms-like tyrosine kinase (Flt-1). The VEGF protein is a heparin-biding protein that induces migration and proliferation of vascular endothelial cells.

Non-limiting examples of protein and nucleotide sequences encoding a wildtype VEGF protein are shown below.

Human VEGF Transcript Variant 1 Protein Sequence
(SEQ ID NO: 1)
MTDRQTDTAPSPSYHLLPGRRRTVDAAASRGQGPEPAPGGGVEGVGARGV
ALKLFVQLLGCSRFGGAVVRAGEAEPSGAARSASSGREEPQPEEGEEEEE
KEEERGPQWRLGARKPGSWTGEAAVCADSAPAARAPQALARASGRGGRVA
RRGAEESGPPHSPSRRGSASRAGPGRASETMNFLLSWVHWSLALLLYLHH
AKWSQAAPMAEGGGQNHHEVVKFMDVYQRSYCHPIETLVDIFQEYPDEIE
YIFKPSCVPLMRCGGCCNDEGLECVPTEESNITMQIMRIKPHQGQHIGEM
SFLQHNKCECRPKKDRARQEKKSVRGKGKGQKRKRKKSRYKSWSVYVGAR
CCLMPWSLPGPHPCGPCSERRKHLFVQDPQTCKCSCKNTDSRCKARQLEL
NERTCRCDKPRR
Human VEGF Transcript Variant 1 cDNA
(SEQ ID NO: 2)
ct gacggacaga cagacagaca ccgcccccag ccccagctac
cacctcctcc ccggccggcg gcggacagtg gacgcggcgg
cgagccgcgg gcaggggccg gagcccgcgc ccggaggcgg
ggtggagggg gtcggggctc gcggcgtcgc actgaaactt
ttcgtccaac ttctgggctg ttctcgcttc ggaggagccg
tggtccgcgc gggggaagccgagccgagcg gagccgcgag
aagtgctagc tcgggccggg aggagccgca gccggaggag
ggggaggagg aagaagagaa ggaagaggag agggggccgc
agtggcgact cggcgctcgg aagccgggct catggacggg
tgaggcggcg gtgtgcgcag acagtgctcc agccgcgcgc
gctccccagg ccctggcccg ggcctcgggc cggggaggaa
gagtagctcg ccgaggcgcc gaggagagcg ggccgcccca
cagcccgagc cggagaggga gcgcgagccg cgccggcccc
ggtcgggcct ccgaaaccat gaactttctg ctgtcttggg
tgcattggag ccttgccttgctgctctacc tccaccatgc
caagtggtcc caggctgcac ccatggcaga aggaggaggg
cagaatcatc acgaagtggt gaagttcatg gatgtctatc
agcgcagcta ctgccatcca atcgagaccc tggtggacat
cttccaggag taccctgatg agatcgagta catcttcaag
ccatcctgtg tgcccctgat gcgatgcggg ggctgctgca
atgacgaggg cctggagtgt gtgcccactg aggagtccaa
catcaccatg cagattatgc ggatcaaacc tcaccaaggc
cagcacatag gagagatgag cttcctacag cacaacaaat
gtgaatgcag accaaagaaa gatagagcaa gacaagaaaa
aaaatcagtt cgaggaaagg gaaaggggca aaaacgaaag
cgcaagaaat cccggtataa gtcctggagc gtgtacgttg
gtgcccgctg ctgtctaatg ccctggagcc tccctggccc
ccatccctgt gggccttgct cagagcggag aaagcatttg
tttgtacaag atccgcagac gtgtaaatgt tcctgcaaaa
acacagactc gcgttgcaag gcgaggcagc ttgagttaaa
cgaacgtact tgcagatgtg acaagccgag gcggtga
Human VEGF Transcript Variant 3 Protein Sequence
(SEQ ID NO: 3)
MTDRQTDTAPSPSYHLLPGRRRTVDAAASRGQGPEPAPGGGVEGVGARGV
ALKLFVQLLGCSRFGGAVVRAGEAEPSGAARSASSGREEPQPEEGEEEEE
KEEERGPQWRLGARKPGSWTGEAAVCADSAPAARAPQALARASGRGGRVA
RRGAEESGPPHSPSRRGSASRAGPGRASETMNFLLSWVHWSLALLLYLHH
AKWSQAAPMAEGGGQNHHEVVKFMDVYQRSYCHPIETLVDIFQEYPDEIE
YIFKPSCVPLMRCGGCCNDEGLECVPTEESNITMQIMRIKPHQGQHIGEM
SFLQHNKCECRPKKDRARQEKKSVRGKGKGQKRKRKKSRPCGPCSERRKH
LFVQDPQTCKCSCKNTDSRCKARQLELNERTCRCDKPRR
Human VEGF Transcript Variant 3 cDNA
(SEQ ID NO: 4)
ct gacggacaga cagacagaca ccgcccccag ccccagctac
cacctcctcc ccggccggcg gcggacagtg gacgcggcgg
cgagccgcgg gcaggggccg gagcccgcgc ccggaggcgg
ggtggagggg gtcggggctc gcggcgtcgc actgaaactt
ttcgtccaac ttctgggctg ttctcgcttc ggaggagccg
tggtccgcgc gggggaagccgagccgagcg gagccgcgag
aagtgctagc tcgggccggg aggagccgca gccggaggag
ggggaggagg aagaagagaa ggaagaggag agggggccgc
agtggcgact cggcgctcgg aagccgggct catggacggg
tgaggcggcg gtgtgcgcag acagtgctcc agccgcgcgc
gctccccagg ccctggcccg ggcctcgggc cggggaggaa
gagtagctcg ccgaggcgcc gaggagagcg ggccgcccca
cagcccgagc cggagaggga gcgcgagccg cgccggcccc
ggtcgggcct ccgaaaccat gaactttctg ctgtcttggg
tgcattggag ccttgccttgctgctctacc tccaccatgc
caagtggtcc caggctgcac ccatggcaga aggaggaggg
cagaatcatc acgaagtggt gaagttcatg gatgtctatc
agcgcagcta ctgccatcca atcgagaccc tggtggacat
cttccaggag taccctgatg agatcgagta catcttcaag
ccatcctgtg tgcccctgat gcgatgcggg ggctgctgca
atgacgaggg cctggagtgt gtgcccactg aggagtccaa
catcaccatg cagattatgc ggatcaaacc tcaccaaggc
cagcacatag gagagatgag cttcctacag cacaacaaat
gtgaatgcag accaaagaaa gatagagcaa gacaagaaaa
aaaatcagtt cgaggaaagg gaaaggggca aaaacgaaag
cgcaagaaat cccgtccctg tgggccttgc tcagagcgga
gaaagcattt gtttgtacaa gatccgcaga cgtgtaaatg
ttcctgcaaa aacacagact cgcgttgcaa ggcgaggcag
cttgagttaa acgaacgtac ttgcagatgt gacaagccga
ggcggtga
Mature Human VEGF-A
(SEQ ID NO: 13)
apma egggqnhhev vkfmdvyqrs ychpietlvd
ifqeypdeie yifkpscvpl mrcggccnde glecvptees
nitmqimrik phqgqhigem sflghnkcec rpkkdrarqe
kksvrgkgkg qkrkrkksry kswsvyvgar cclmpwslpg
phpcgpcser rkhlfvqdpq tckcsckntd srckarqlel
nertcrcdkp rr
Mature Human VEGF-B
(SEQ ID NO: 14)
pvsqpdapg hqrkvvswid vytratcqpr evvvpltvel
mgtvakqlvp scvtvqrcgg ccpddglecv ptgqhqvrmq
ilmirypssq lgemsleehs qcecrpkkkd savkpdraat
phhrpqprsv pgwdsapgap spadithptp apgpsahaap
sttsaltpgp aaaaadaaas svakgga
Mature Human VEGF-C
(SEQ ID NO: 15)
Ahynteilk sidnewrktq cmprevcidv gkefgvatnt
ffkppcvsvy rcggccnseg lqcmntstsy lsktifeitv
plsqgpkpvt isfanhtscr cmskldvyrq vhsiirr
Mature Human VEGF-D
(SEQ ID NO: 16)
fa atfydietlk videewqrtq cspretcvev aselgkstnt
ffkppcvnvf rcggccnees licmntstsy iskqlfeisv
pltsvpelvp vkvanhtgck clptaprhpy siirr

In some examples of any of the antibodies and antigen-binding fragments thereof described herein, the antibody and antigen-binding fragment can bind to a VEGF antigen (e.g., any of the exemplary VEGF proteins described herein, e.g., one or more of mature human VEGF-A, mature human VEGF-B, mature human VEGF-C, and mature human VEGF-D) (e.g., any of the binding affinities described herein).

In some embodiments described herein, an antibody or antigen-binding antibody fragment can decrease an activity of a VEGF (e.g., one or more of any of the exemplary VEGF proteins described herein, e.g., one or more of mature human VEGF-A, mature human VEGF-B, mature human VEGF-C, and mature human VEGF-D). In some embodiments, an antibody or antigen-binding antibody fragment can block a VEGF (e.g., one or more of any of the exemplary VEGF proteins described herein, e.g., one or more of mature human VEGF-A, mature human VEGF-B, mature human VEGF-C, and mature human VEGF-D) from binding to one or more of its receptors (e.g., one or more VEGF receptors) See, e.g., WO 1998/045331, U.S. Pat. No. 9,079,953, U.S. 2015/0147317, U.S. 2016/0289314, Plotkin et al., Otology & Neurotology 33: 1046-1052 (2012); and Ferrara et al. (2005) Biochem Biophys Res Commun 333(2): 328-335. In some embodiments, an antibody or antigen-binding antibody can decrease downstream signaling (e.g., signaling downstream of a VEGF receptor, e.g., one or more of any of the exemplary VEGF receptors described herein, e.g., one or more of human VEGFR-1, human VEGFR-2, and human VEGFR-3). In some embodiments, a decrease in an activity of a VEGF can be detected indirectly, e.g., through an increase in hearing (e.g., a 1% to about 400% increase (or any of the subranges of this range described herein) in hearing) or a decrease (e.g., a 1% to 99%, a 1% to 95%, a 1% to 90%, a 1% to 85%, a 1% to 80%, a 1% to 75%, a 1% to 70%, a 1% to 65%, a 1% to 60%, a 1% to 55%, a 1% to 50%, a 1% to 45%, a 1% to 40%, a 1% to 35%, a 1% to 30%, a 1% to 25%, a 1% to 20%, a 1% to 15%, a 1% to 10%, a 1% to 5%, a 5% to 99%, a 5% to 95%, a 5% to 90%, a 5% to 85%, a 5% to 80%, a 5% to 75%, a 5% to 70%, a 5% to 65%, a 5% to 60%, a 5% to 55%, a 5% to 50%, a 5% to 45%, a 5% to 40%, a 5% to 35%, a 5% to 30%, a 5% to 25%, a 5% to 20%, a 5% to 15%, a 5% to 10%, a 10% to 99%, a 10% to 95%, a 10% to 90%, a 10% to 85%, a 10% to 80%, a 10% to 75%, a 10% to 70%, a 10% to 65%, a 10% to 60%, a 10% to 55%, a 10% to 50%, a 10% to 45%, a 10% to 40%, a 10% to 35%, a 10% to 30%, a 10% to 25%, a 10% to 20%, a 10% to 15%, a 15% to 99%, a 15% to 95%, a 15% to 90%, a 15% to 85%, a 15% to 80%, a 15% to 75%, a 15% to 70%, a 15% to 65%, a 15% to 60%, a 15% to 55%, a 15% to 50%, a 15% to 45%, a 15% to 40%, a 15% to 35%, a 15% to 30%, a 15% to 25%, a 15% to 20%, a 20% to 99%, a 20% to 95%, a 20% to 90%, a 20% to 85%, a 20% to 80%, a 20% to 75%, a 20% to 70%, a 20% to 65%, a 20% to 60%, a 20% to 55%, a 20% to 50%, a 20% to 45%, a 20% to 40%, a 20% to 35%, a 20% to 30%, a 20% to 25%, a 25% to 99%, a 25% to 95%, a 25% to 90%, a 25% to 85%, a 25% to 80%, a 25% to 75%, a 25% to 70%, a 25% to 65%, a 25% to 60%, a 25% to 55%, a 25% to 50%, a 25% to 45%, a 25% to 40%, a 25% to 35%, a 25% to 30%, a 30% to 99%, a 30% to 95%, a 30% to 90%, a 30% to 85%, a 30% to 80%, a 30% to 75%, a 30% to 70%, a 30% to 65%, a 30% to 60%, a 30% to 55%, a 30% to 50%, a 30% to 45%, a 30% to 40%, a 30% to 35%, a 35% to 99%, a 35% to 95%, a 35% to 90%, a 35% to 85%, a 35% to 80%, a 35% to 75%, a 35% to 70%, a 35% to 65%, a 35% to 60%, a 35% to 55%, a 35% to 50%, a 35% to 45%, a 35% to 40%, a 40% to 99%, a 40% to 95%, a 40% to 90%, a 40% to 85%, a 40% to 80%, a 40% to 75%, a 40% to 70%, a 40% to 65%, a 40% to 60%, a 40% to 55%, a 40% to 50%, a 40% to 45%, a 45% to 99%, a 45% to 95%, a 45% to 90%, a 45% to 85%, a 45% to 80%, a 45% to 75%, a 45% to 70%, a 45% to 65%, a 45% to 60%, a 45% to 55%, a 45% to 50%, a 50% to 99%, a 50% to 95%, a 50% to 90%, a 50% to 85%, a 50% to 80%, a 50% to 75%, a 50% to 70%, a 50% to 65%, a 50% to 60%, a 50% to 55%, a 55% to 99%, a 55% to 95%, a 55% to 90%, a 55% to 85%, a 55% to 80%, a 55% to 75%, a 55% to 70%, a 55% to 65%, a 55% to 60%, a 60% to 99%, a 60% to 95%, a 60% to 90%, a 60% to 85%, a 60% to 80%, a 60% to 75%, a 60% to 70%, a 60% to 65%, a 65% to 99%, a 65% to 95%, a 65% to 90%, a 65% to 85%, a 65% to 80%, a 65% to 75%, a 65% to 70%, a 70% to 99%, a 70% to 95%, a 70% to 90%, a 70% to 85%, a 70% to 80%, a 70% to 75%, a 75% to 99%, a 75% to 95%, a 75% to 90%, a 75% to 85%, a 75% to 80%, a 80% to 99%, a 80% to 95%, a 80% to 90%, a 80% to 85%, a 85% to 99%, a 85% to 95%, a 85% to 90%, a 90% to 99%, a 90% to 95%, or a 95% to 99% decrease) in the size or the severity of one or more symptoms of an acoustic neuroma, vestibular schwannoma, or neurofibromatosis type II in a mammal as compared to the level of hearing or size of an acoustic neuroma, vestibular schwannoma, or neurofibromatosis type II in the mammal, respectively, before administration of any of the AAV vectors described herein. In some embodiments, a decrease in a VEGF activity can be detected in an in vitro assay.

In some embodiments, the antibody that specifically binds to a VEGF is bevacizumab (Avastatin®) or an antigen-binding fragment thereof. Bevacizumab (full size antibody ˜150 kDa) inhibits all isoforms of VEGF-A. Bevacizumab received Food and Drug administration (FDA) approval in 2004 for colon cancer for intravenous (IV) dose of 4.0-7.5 mg/kg at 2-3 weeks (plasmatic half life 21 days), for intravetrial (IVT) dose 1.25 mg in 0.05 mL (half-life 5.6 days). Bevacizumab has a K_{D} for VEGF 165 (VEGF-A) of 58 pM. See, e.g., WO 2017/050825. In some embodiments, the antibody that specifically binds to a VEGF is ranibizumab (Lucentis®), or an antigen-binding fragment thereof. Ranibizumab (˜50 kDa) inhibits all isoforms of VEGF-A. Ranibizumab received FDA approval in 2006 for ocular use for intravenous (IV) dose of 4.0-7.5 mg/kg at 2-3 weeks (plasma half life of 0.5 days), for intravetrial (IVT) dose 0.5 mg in 0.05 mL (half-life of 3.2 days). Ranibizumab has a K_{D} for VEGF 165 (VEGF-A) of 46 pM. See, e.g., WO 2014/178078. In some embodiments, the antibody that specifically binds to VEGF is sevacizumab (APX003/SIM-BD0801), or an antigen-binding fragment thereof

Amino Acid Encoding Light Chain of
Bevacizumab
(SEQ ID NO: 5)
DIQMTQSPSSLSASVGDRVTITCSASQDISNYLNWYQQKPGKAPKVLIYF
TSSLHSGVPSRFSGSGSGTDFTLTISSLQPEDFATYYCQQYSTVPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Amino Acid Encoding Heavy Chain of
Bevacizumab
(SEQ ID NO: 6)
EVQLVESGGGLVQPGGSLRLSCAASGYTFTNYGMNWVRQAPGKGLEWVGW
INTYTGEPTYAADFKRRFTFSLDTSKSTAYLQMNSLRAEDTAVYYCAKYP
HYYGSSHWYFDVWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK

In some embodiments of the antibodies that specifically bind to VEGF and antigen-binding fragments thereof described herein, the antibody or antigen-binding fragments thereof includes a variable light chain domain that is or includes a sequence that is at least 80% identical (e.g., at least 82%, at least 84%, at least 86%, at least 88%, at least 90%, at least 92%, at least 94%, at least 96%, at least 98%, or at least 99%) identical to the variable light chain domain of bevacizumab, and/or includes a variable heavy chain domain that is or includes a sequence that is at least 80% identical (e.g., at least 82%, at least 84%, at least 86%, at least 88%, at least 90%, at least 92%, at least 94%, at least 96%, at least 98%, or at least 99%) identical to the variable heavy chain domain of bevacizumab.

In some embodiments of the antibodies that specifically bind to VEGF and antigen-binding fragments thereof described herein, the antibody or antigen-binding fragments thereof includes a variable light chain domain that is or includes the variable light chain domain of bevacizumab, and/or a variable heavy chain domain that is or includes the variable heavy chain domain of bevacizumab. In some embodiments of the antibodies that specifically bind to VEGF and antigen-binding fragments thereof described herein, the antibody or antigen-binding fragments thereof includes a variable light chain domain that is or includes the sequence of variable light chain domain of bevacizumab, except that it includes one, two, three, four, five, six, seven, eight, nine, ten, eleven, twelve, thirteen, fourteen, or fifteen amino acid substitutions, and/or includes a variable heavy chain domain that is or includes the sequence of variable heavy chain of bevacizumab, except that it includes one, two, three, four, five, six, seven, eight, nine, ten, eleven, twelve, thirteen, fourteen, or fifteen amino acid substitutions. In some embodiments the first antigen-binding domain includes the three CDRs in the light chain variable domain of bevacizumab, and/or the three CDRs in the heavy chain variable domain of bevacizumab.

Amino Acid Encoding Light Chain of
Ranibizumab
(SEQ ID NO: 7)
DIQLTQSPSSLSASVGDRVTITCSASQDISNYLNWYQQKPGKAPKVLIYF
TSSLHSGVPSRFSGSGSGTDFTLTISSLQPEDFATYYCQQYSTVPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Amino Acid Encoding Heavy Chain of
Ranibizumab
(SEQ ID NO: 8)
EVQLVESGGGLVQPGGSLRLSCAASGYDFTHYGMNWVRQAPGKGLEWVGW
INTYTGEPTYAADFKRRFTFSLDTSKSTAYLQMNSLRAEDTAVYYCAKYP
YYYGTSHWYFDVWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHL

In some embodiments of the antibodies that specifically bind to VEGF and antigen-binding fragments thereof described herein, the antibody or antigen-binding fragments thereof includes a variable light chain domain that is or includes a sequence that is at least 80% identical (e.g., at least 82%, at least 84%, at least 86%, at least 88%, at least 90%, at least 92%, at least 94%, at least 96%, at least 98%, or at least 99%) identical to the variable light chain domain of ranibizumab, and/or includes a variable heavy chain domain that is or includes a sequence that is at least 80% identical (e.g., at least 82%, at least 84%, at least 86%, at least 88%, at least 90%, at least 92%, at least 94%, at least 96%, at least 98%, or at least 99%) identical to the variable heavy chain domain of ranibizumab.

In some embodiments of the antibodies that specifically bind to VEGF and antigen-binding fragments thereof described herein, the antibody or antigen-binding fragments thereof includes a variable light chain domain that is or includes the variable light chain domain of ranibizumab, and/or a variable heavy chain domain that is or includes the variable heavy chain domain of ranibizumab. In some embodiments of the antibodies that specifically bind to VEGF and antigen-binding fragments thereof described herein, the antibody or antigen-binding fragments thereof includes a variable light chain domain that is or includes the sequence of variable light chain domain of ranibizumab, except that it includes one, two, three, four, five, six, seven, eight, nine, ten, eleven, twelve, thirteen, fourteen, or fifteen amino acid substitutions, and/or includes a variable heavy chain domain that is or includes the sequence of variable heavy chain of ranibizumab, except that it includes one, two, three, four, five, six, seven, eight, nine, ten, eleven, twelve, thirteen, fourteen, or fifteen amino acid substitutions. In some embodiments the first antigen-binding domain includes the three CDRs in the light chain variable domain of ranibizumab, and/or the three CDRs in the heavy chain variable domain of ranibizumab.

### Soluble VEGF Receptors

A soluble VEGF receptor is a polypeptide that includes a portion of an extracellular region of one or more (e.g., two or three) mammalian VEGF receptor(s) (e.g., one or more of VEGFR-1, VEGFR-2, and VEGFR-3) operably linked to a signal peptide (e.g., any of the exemplary signal peptides described herein), where the soluble VEGF receptor is capable of specifically binding to one or more mammalian VEGF protein(s) (e.g., one or more (e.g., two, three, or four) of VEGF-A, VEGF-B, VEGF-C, and VEGF-D, e.g., one or more (e.g., two, three, or four) of human wildtype VEGF-A, human wildtype VEGF-B, human wildtype VEGF-C, and human wildtype VEGF-D).

In some examples, a soluble VEGF receptor includes a portion (e.g., about 10 amino acids to about 732 amino acids, about 10 amino acids to about 700 amino acids, about 10 amino acids to about 650 amino acids, about 10 amino acids to about 600 amino acids, about 10 amino acids to about 550 amino acids, about 10 amino acids to about 500 amino acids, about 10 amino acids to about 450 amino acids, about 10 amino acids to about 400 amino acids, about 10 amino acids to about 350 amino acids, about 10 amino acids to about 300 amino acids, about 10 amino acids to about 250 amino acids, about 10 amino acids to about 200 amino acids, about 10 amino acids to about 150 amino acids, about 10 amino acids to about 100 amino acids, about 10 amino acids to about 50 amino acids, about 50 amino acids to about 732 amino acids, about 50 amino acids to about 700 amino acids, about 50 amino acids to about 650 amino acids, about 50 amino acids to about 600 amino acids, about 50 amino acids to about 550 amino acids, about 50 amino acids to about 500 amino acids, about 50 amino acids to about 450 amino acids, about 50 amino acids to about 400 amino acids, about 50 amino acids to about 350 amino acids, about 50 amino acids to about 300 amino acids, about 50 amino acids to about 250 amino acids, about 50 amino acids to about 200 amino acids, about 50 amino acids to about 150 amino acids, about 50 amino acids to about 100 amino acids, about 100 amino acids to about 732 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 450 amino acids, about 100 amino acids to about 400 amino acids, about 100 amino acids to about 350 amino acids, about 100 amino acids to about 300 amino acids, about 100 amino acids to about 250 amino acids, about 100 amino acids to about 200 amino acids, about 100 amino acids to about 150 amino acids, about 150 amino acids to about 732 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 450 amino acids, about 150 amino acids to about 400 amino acids, about 150 amino acids to about 350 amino acids, about 150 amino acids to about 300 amino acids, about 150 amino acids to about 250 amino acids, about 150 amino acids to about 200 amino acids, about 200 amino acids to about 732 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 450 amino acids, about 200 amino acids to about 400 amino acids, about 200 amino acids to about 350 amino acids, about 200 amino acids to about 300 amino acids, about 200 amino acids to about 250 amino acids, about 250 amino acids to about 732 amino acids, about 250 amino acids to about 700 amino acids, about 250 amino acids to about 650 amino acids, about 250 amino acids to about 600 amino acids, about 250 amino acids to about 550 amino acids, about 250 amino acids to about 500 amino acids, about 250 amino acids to about 450 amino acids, about 250 amino acids to about 400 amino acids, about 250 amino acids to about 350 amino acids, about 250 amino acids to about 300 amino acids, about 300 amino acids to about 732 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 450 amino acids, about 300 amino acids to about 400 amino acids, about 300 amino acids to about 350 amino acids, about 350 amino acids to about 732 amino acids, about 350 amino acids to about 700 amino acids, about 350 amino acids to about 650 amino acids, about 350 amino acids to about 600 amino acids, about 350 amino acids to about 550 amino acids, about 350 amino acids to about 500 amino acids, about 350 amino acids to about 450 amino acids, about 350 amino acids to about 400 amino acids, about 400 amino acids to about 732 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 450 amino acids, about 450 amino acids to about 732 amino acids, about 450 amino acids to about 700 amino acids, about 450 amino acids to about 650 amino acids, about 450 amino acids to about 600 amino acids, about 450 amino acids to about 550 amino acids, about 450 amino acids to about 500 amino acids, about 500 amino acids to about 732 amino acids, about 500 amino acids to about 700 amino acids, about 500 amino acids to about 650 amino acids, about 500 amino acids to about 600 amino acids, about 500 amino acids to about 550 amino acids, about 550 amino acids to about 732 amino acids, about 550 amino acids to about 700 amino acids, about 550 amino acids to about 650 amino acids, about 550 amino acids to about 600 amino acids, about 600 amino acids to about 732 amino acids, about 600 amino acids to about 700 amino acids, about 600 amino acids to about 650 amino acids, about 650 amino acids to about 732 amino acids, about 650 amino acids to about 700 amino acids, or about 700 amino acids to about 732 amino acids) of an extracellular region of VEGFR-1 (e.g., a contiguous sequence from wildtype human VEGFR-1 (e.g., a contiguous sequence including one or more (e.g., one, two, three, four, five, six, or seven) immunoglobulin-like domains in the extracellular region from wildtype human VEGFR-1 (e.g., SEQ ID NO: 23) or a sequence that is at least 80% (e.g., at least 82%, at least 84%, at least 86%, at least 88%, at least 90%, at least 92%, at least 94%, at least 96%, at least 98%, or at least 99%) identical to a contiguous sequence from wildtype human VEGFR-1, e.g., a sequence that is at least 80% (e.g., least 82%, at least 84%, at least 86%, at least 88%, at least 90%, least 92%, at least 94%, at least 96%, at least 98%, or at least 99%) identical to a contiguous sequence in SEQ ID NO: 23).

In some examples, a soluble VEGF receptor includes a portion (e.g., about 20 amino acids to about 745 amino acids, or any of the subranges of this range described herein) of an extracellular region of VEGFR-2 (e.g., a contiguous sequence from wildtype human VEGFR-2 (e.g., a contiguous sequence including one or more (e.g., one, two, three, four, five, six, or seven) immunoglobulin-like domains in the extracellular region from wildtype human VEGFR-2 (e.g., SEQ ID NO: 26) or a sequence that is at least 80% (e.g., at least 82%, at least 84%, at least 86%, at least 88%, at least 90%, at least 92%, at least 94%, at least 96%, at least 98%, or at least 99%) identical to a contiguous sequence from wildtype human VEGFR-2, e.g., a sequence that is at least 80% (e.g., at least 82%, at least 84%, at least 86%, at least 88%, at least 90%, at least 92%, at least 94%, at least 96%, at least 98%, or at least 99%) identical to a contiguous sequence in SEQ ID NO: 26).

In some examples, a soluble VEGF receptor includes a portion of an extracellular region of VEGFR-1 (e.g., any of the portions of an extracellular region of VEGFR-1 described herein) and a portion of an extracellular region of VEGFR-2 (e.g., any of the portions of an extracellular region of VEGFR-2 described herein). For example, a soluble VEGF receptor can include one or more (e.g., two, three, four, five, six, or seven) immunoglobulin-like domains in the extracellular region from wildtype human VEGFR-1 and one or more (e.g., two, three, four, five, six, or seven) immunoglobulin-like domains in the extracellular region from wildtype human VEGFR-2 (e.g., aflibercept).

In some examples, a soluble VEGF receptor includes a portion (e.g., about 20 amino acids to about 751 amino acids, or any of the subranges of this range described herein) of an extracellular region of VEGFR-3 (e.g., a contiguous sequence from wildtype human VEGFR-3 (e.g., a contiguous sequence including one or more (e.g., one, two, three, four, five, six, or seven) immunoglobulin-like domains in the extracellular region from wildtype human VEGFR-3 (e.g., SEQ ID NO: 29) or a sequence that is at least 80% (e.g., at least 82%, at least 84%, at least 86%, at least 88%, at least 90%, at least 92%, at least 94%, at least 96%, at least 98%, or at least 99%) identical to a contiguous sequence from wildtype human VEGFR-3, e.g., a sequence that is at least 80% (e.g., at least 82%, at least 84%, at least 86%, at least 88%, at least 90%, at least 92%, at least 94%, at least 96%, at least 98%, or at least 99%) identical to a contiguous sequence in SEQ ID NO: 29).

Non-limiting examples of extracellular regions of different mammalian VEGFR-1, different mammalian VEGFR-2, and different mammalian VEGFR-3 are described herein. Non-limiting examples of protein and nucleotide sequences encoding a wildtype VEGF receptor protein are shown below. As one skilled in the art can appreciate, a substitution in an amino acid that is conserved between species is more likely to result in a change in the function of a protein, while a substitution in an amino acid position that is not converted between species is less likely to have an affect on the function of a protein.

Human VEGF Receptor 1 Isoform 2 Protein
Sequence
(SEQ ID NO: 17)
MVSYWDTGVLLCALLSCLLLTGSSSGSKLKDPELSLKGTQHIMQAGQTLH
LQCRGEAAHKWSLPEMVSKESERLSITKSACGRNGKQFCSTLTLNTAQAN
HTGFYSCKYLAVPTSKKKETESAIYIFISDTGRPFVEMYSEIPEIIHMTE
GRELVIPCRVTSPNITVTLKKFPLDTLIPDGKRIIWDSRKGFIISNATYK
EIGLLTCEATVNGHLYKTNYLTHRQTNTIIDVQISTPRPVKLLRGHTLVL
NCTATTPLNTRVQMTWSYPDEKNKRASVRRRIDQSNSHANIFYSVLTIDK
MQNKDKGLYTCRVRSGPSFKSVNTSVHIYDKAFITVKHRKQQVLETVAGK
RSYRLSMKVKAFPSPEVVWLKDGLPATEKSARYLTRGYSLIIKDVTEEDA
GNYTILLSIKQSNVFKNLTATLIVNVKPQIYEKAVSSFPDPALYPLGSRQ
ILTCTAYGIPQPTIKWFWHPCNHNHSEARCDFCSNNEESFILDADSNMGN
RIESITQRMAIIEGKNKMASTLVVADSRISGIYICIASNKVGTVGRNISF
YITDVPNGFHVNLEKMPTEGEDLKLSCTVNKFLYRDVTWILLRTVNNRTM
HYSISKQKMAITKEHSITLNLTIMNVSLQDSGTYACRARNVYTGEEILQK
KEITIRGEHCNKKAVFSRISKFKSTRNDCTTQSNVKH
Human VEGF Receptor 1 Isoform 2 cDNA
(SEQ ID NO: 18)
ATGGTCAGCTACTGGGACACCGGGGTCCTGCTGTGCGCGCTGCTCAGCTG
TCTGCTTCTCACAGGATCTAGTTCAGGTTCAAAATTAAAAGATCCTGAAC
TGAGTTTAAAAGGCACCCAGCACATCATGCAAGCAGGCCAGACACTGCAT
CTCCAATGCAGGGGGGAAGCAGCCCATAAATGGTCTTTGCCTGAAATGGT
GAGTAAGGAAAGCGAAAGGCTGAGCATAACTAAATCTGCCTGTGGAAGAA
ATGGCAAACAATTCTGCAGTACTTTAACCTTGAACACAGCTCAAGCAAAC
CACACTGGCTTCTACAGCTGCAAATATCTAGCTGTACCTACTTCAAAGAA
GAAGGAAACAGAATCTGCAATCTATATATTTATTAGTGATACAGGTAGAC
CTTTCGTAGAGATGTACAGTGAAATCCCCGAAATTATACACATGACTGAA
GGAAGGGAGCTCGTCATTCCCTGCCGGGTTACGTCACCTAACATCACTGT
TACTTTAAAAAAGTTTCCACTTGACACTTTGATCCCTGATGGAAAACGCA
TAATCTGGGACAGTAGAAAGGGCTTCATCATATCAAATGCAACGTACAAA
GAAATAGGGCTTCTGACCTGTGAAGCAACAGTCAATGGGCATTTGTATAA
GACAAACTATCTCACACATCGACAAACCAATACAATCATAGATGTCCAAA
TAAGCACACCACGCCCAGTCAAATTACTTAGAGGCCATACTCTTGTCCTC
AATTGTACTGCTACCACTCCCTTGAACACGAGAGTTCAAATGACCTGGAG
TTACCCTGATGAAAAAAATAAGAGAGCTTCCGTAAGGCGACGAATTGACC
AAAGCAATTCCCATGCCAACATATTCTACAGTGTTCTTACTATTGACAAA
ATGCAGAACAAAGACAAAGGACTTTATACTTGTCGTGTAAGGAGTGGACC
ATCATTCAAATCTGTTAACACCTCAGTGCATATATATGATAAAGCATTCA
TCACTGTGAAACATCGAAAACAGCAGGTGCTTGAAACCGTAGCTGGCAAG
CGGTCTTACCGGCTCTCTATGAAAGTGAAGGCATTTCCCTCGCCGGAAGT
TGTATGGTTAAAAGATGGGTTACCTGCGACTGAGAAATCTGCTCGCTATT
TGACTCGTGGCTACTCGTTAATTATCAAGGACGTAACTGAAGAGGATGCA
GGGAATTATACAATCTTGCTGAGCATAAAACAGTCAAATGTGTTTAAAAA
CCTCACTGCCACTCTAATTGTCAATGTGAAACCCCAGATTTACGAAAAGG
CCGTGTCATCGTTTCCAGACCCGGCTCTCTACCCACTGGGCAGCAGACAA
ATCCTGACTTGTACCGCATATGGTATCCCTCAACCTACAATCAAGTGGTT
CTGGCACCCCTGTAACCATAATCATTCCGAAGCAAGGTGTGACTTTTGTT
CCAATAATGAAGAGTCCTTTATCCTGGATGCTGACAGCAACATGGGAAAC
AGAATTGAGAGCATCACTCAGCGCATGGCAATAATAGAAGGAAAGAATAA
GATGGCTAGCACCTTGGTTGTGGCTGACTCTAGAATTTCTGGAATCTACA
TTTGCATAGCTTCCAATAAAGTTGGGACTGTGGGAAGAAACATAAGCTTT
TATATCACAGATGTGCCAAATGGGTTTCATGTTAACTTGGAAAAAATGCC
GACGGAAGGAGAGGACCTGAAACTGTCTTGCACAGTTAACAAGTTCTTAT
ACAGAGACGTTACTTGGATTTTACTGCGGACAGTTAATAACAGAACAATG
CACTACAGTATTAGCAAGCAAAAAATGGCCATCACTAAGGAGCACTCCAT
CACTCTTAATCTTACCATCATGAATGTTTCCCTGCAAGATTCAGGCACCT
ATGCCTGCAGAGCCAGGAATGTATACACAGGGGAAGAAATCCTCCAGAAG
AAAGAAATTACAATCAGAGGTGAGCACTGCAACAAAAAGGCTGTTTTCTC
TCGGATCTCCAAATTTAAAAGCACAAGGAATGATTGTACCACACAAAGTA
ATGTAAAACATTAA
Human VEGF Receptor 1 Isoform 3 Protein
Sequence (sFlt1-14)
(SEQ ID NO: 19)
MVSYWDTGVLLCALLSCLLLTGSSSGSKLKDPELSLKGTQHIMQAGQTLH
LQCRGEAAHKWSLPEMVSKESERLSITKSACGRNGKQFCSTLTLNTAQAN
HTGFYSCKYLAVPTSKKKETESAIYIFISDTGRPFVEMYSEIPEIIHMTE
GRELVIPCRVTSPNITVTLKKFPLDTLIPDGKRIIWDSRKGFIISNATYK
EIGLLTCEATVNGHLYKTNYLTHRQTNTIIDVQISTPRPVKLLRGHTLVL
NCTATTPLNTRVQMTWSYPDEKNKRASVRRRIDQSNSHANIFYSVLTIDK
MQNKDKGLYTCRVRSGPSFKSVNTSVHIYDKAFITVKHRKQQVLETVAGK
RSYRLSMKVKAFPSPEVVWLKDGLPATEKSARYLTRGYSLIIKDVTEEDA
GNYTILLSIKQSNVFKNLTATLIVNVKPQIYEKAVSSFPDPALYPLGSRQ
ILTCTAYGIPQPTIKWFWHPCNHNHSEARCDFCSNNEESFILDADSNMGN
RIESITQRMAIIEGKNKMASTLVVADSRISGIYICIASNKVGTVGRNISF
YITDVPNGFHVNLEKMPTEGEDLKLSCTVNKFLYRDVTWILLRTVNNRTM
HYSISKQKMAITKEHSITLNLTIMNVSLQDSGTYACRARNVYTGEEILQK
KEITIRDQEAPYLLRNLSDHTVAISSSTTLDCHANGVPEPQITWFKNNHK
IQQEPELYTSTSPSSSSSSPLSSSSSSSSSSSS
Human VEGF Receptor 1 Isoform 3 cDNA
(SEQ ID NO: 20)
ATGGTCAGCTACTGGGACACCGGGGTCCTGCTGTGCGCGCTGCTCAGCTG
TCTGCTTCTCACAGGATCTAGTTCAGGTTCAAAATTAAAAGATCCTGAAC
TGAGTTTAAAAGGCACCCAGCACATCATGCAAGCAGGCCAGACACTGCAT
CTCCAATGCAGGGGGGAAGCAGCCCATAAATGGTCTTTGCCTGAAATGGT
GAGTAAGGAAAGCGAAAGGCTGAGCATAACTAAATCTGCCTGTGGAAGAA
ATGGCAAACAATTCTGCAGTACTTTAACCTTGAACACAGCTCAAGCAAAC
CACACTGGCTTCTACAGCTGCAAATATCTAGCTGTACCTACTTCAAAGAA
GAAGGAAACAGAATCTGCAATCTATATATTTATTAGTGATACAGGTAGAC
CTTTCGTAGAGATGTACAGTGAAATCCCCGAAATTATACACATGACTGAA
GGAAGGGAGCTCGTCATTCCCTGCCGGGTTACGTCACCTAACATCACTGT
TACTTTAAAAAAGTTTCCACTTGACACTTTGATCCCTGATGGAAAACGCA
TAATCTGGGACAGTAGAAAGGGCTTCATCATATCAAATGCAACGTACAAA
GAAATAGGGCTTCTGACCTGTGAAGCAACAGTCAATGGGCATTTGTATAA
GACAAACTATCTCACACATCGACAAACCAATACAATCATAGATGTCCAAA
TAAGCACACCACGCCCAGTCAAATTACTTAGAGGCCATACTCTTGTCCTC
AATTGTACTGCTACCACTCCCTTGAACACGAGAGTTCAAATGACCTGGAG
TTACCCTGATGAAAAAAATAAGAGAGCTTCCGTAAGGCGACGAATTGACC
AAAGCAATTCCCATGCCAACATATTCTACAGTGTTCTTACTATTGACAAA
ATGCAGAACAAAGACAAAGGACTTTATACTTGTCGTGTAAGGAGTGGACC
ATCATTCAAATCTGTTAACACCTCAGTGCATATATATGATAAAGCATTCA
TCACTGTGAAACATCGAAAACAGCAGGTGCTTGAAACCGTAGCTGGCAAG
CGGTCTTACCGGCTCTCTATGAAAGTGAAGGCATTTCCCTCGCCGGAAGT
TGTATGGTTAAAAGATGGGTTACCTGCGACTGAGAAATCTGCTCGCTATT
TGACTCGTGGCTACTCGTTAATTATCAAGGACGTAACTGAAGAGGATGCA
GGGAATTATACAATCTTGCTGAGCATAAAACAGTCAAATGTGTTTAAAAA
CCTCACTGCCACTCTAATTGTCAATGTGAAACCCCAGATTTACGAAAAGG
CCGTGTCATCGTTTCCAGACCCGGCTCTCTACCCACTGGGCAGCAGACAA
ATCCTGACTTGTACCGCATATGGTATCCCTCAACCTACAATCAAGTGGTT
CTGGCACCCCTGTAACCATAATCATTCCGAAGCAAGGTGTGACTTTTGTT
CCAATAATGAAGAGTCCTTTATCCTGGATGCTGACAGCAACATGGGAAAC
AGAATTGAGAGCATCACTCAGCGCATGGCAATAATAGAAGGAAAGAATAA
GATGGCTAGCACCTTGGTTGTGGCTGACTCTAGAATTTCTGGAATCTACA
TTTGCATAGCTTCCAATAAAGTTGGGACTGTGGGAAGAAACATAAGCTTT
TATATCACAGATGTGCCAAATGGGTTTCATGTTAACTTGGAAAAAATGCC
GACGGAAGGAGAGGACCTGAAACTGTCTTGCACAGTTAACAAGTTCTTAT
ACAGAGACGTTACTTGGATTTTACTGCGGACAGTTAATAACAGAACAATG
CACTACAGTATTAGCAAGCAAAAAATGGCCATCACTAAGGAGCACTCCAT
CACTCTTAATCTTACCATCATGAATGTTTCCCTGCAAGATTCAGGCACCT
ATGCCTGCAGAGCCAGGAATGTATACACAGGGGAAGAAATCCTCCAGAAG
AAAGAAATTACAATCAGAGATCAGGAAGCACCATACCTCCTGCGAAACCT
CAGTGATCACACAGTGGCCATCAGCAGTTCCACCACTTTAGACTGTCATG
CTAATGGTGTCCCCGAGCCTCAGATCACTTGGTTTAAAAACAACCACAAA
ATACAACAAGAGCCTGAACTGTATACATCAACGTCACCATCGTCATCGTC
ATCATCACCATTGTCATCATCATCATCATCGTCATCATCATCATCATCAT
AG
Human VEGF Receptor 1 Isoform 4 Protein
Sequence
(SEQ ID NO: 21)
MVSYWDTGVLLCALLSCLLLTGSSSGSKLKDPELSLKGTQHIMQAGQTLH
LQCRGEAAHKWSLPEMVSKESERLSITKSACGRNGKQFCSTLTLNTAQAN
HTGFYSCKYLAVPTSKKKETESAIYIFISDTGRPFVEMYSEIPEIIHMTE
GRELVIPCRVTSPNITVTLKKFPLDTLIPDGKRIIWDSRKGFIISNATYK
EIGLLTCEATVNGHLYKTNYLTHRQTNTIIDVQISTPRPVKLLRGHTLVL
NCTATTPLNTRVQMTWSYPDEKNKRASVRRRIDQSNSHANIFYSVLTIDK
MQNKDKGLYTCRVRSGPSFKSVNTSVHIYDKAFITVKHRKQQVLETVAGK
RSYRLSMKVKAFPSPEVVWLKDGLPATEKSARYLTRGYSLIIKDVTEEDA
GNYTILLSIKQSNVFKNLTATLIVNVKPQIYEKAVSSFPDPALYPLGSRQ
ILTCTAYGIPQPTIKWFWHPCNHNHSEARCDFCSNNEESFILDADSNMGN
RIESITQRMAIIEGKNKLPPANSSFMLPPTSFSSNYFHFLP
Human VEGF Receptor 1 Isoform 4 cDNA
(SEQ ID NO: 22)
ATGGTCAGCTACTGGGACACCGGGGTCCTGCTGTGCGCGCTGCTCAGCTG
TCTGCTTCTCACAGGATCTAGTTCAGGTTCAAAATTAAAAGATCCTGAAC
TGAGTTTAAAAGGCACCCAGCACATCATGCAAGCAGGCCAGACACTGCAT
CTCCAATGCAGGGGGGAAGCAGCCCATAAATGGTCTTTGCCTGAAATGGT
GAGTAAGGAAAGCGAAAGGCTGAGCATAACTAAATCTGCCTGTGGAAGAA
ATGGCAAACAATTCTGCAGTACTTTAACCTTGAACACAGCTCAAGCAAAC
CACACTGGCTTCTACAGCTGCAAATATCTAGCTGTACCTACTTCAAAGAA
GAAGGAAACAGAATCTGCAATCTATATATTTATTAGTGATACAGGTAGAC
CTTTCGTAGAGATGTACAGTGAAATCCCCGAAATTATACACATGACTGAA
GGAAGGGAGCTCGTCATTCCCTGCCGGGTTACGTCACCTAACATCACTGT
TACTTTAAAAAAGTTTCCACTTGACACTTTGATCCCTGATGGAAAACGCA
TAATCTGGGACAGTAGAAAGGGCTTCATCATATCAAATGCAACGTACAAA
GAAATAGGGCTTCTGACCTGTGAAGCAACAGTCAATGGGCATTTGTATAA
GACAAACTATCTCACACATCGACAAACCAATACAATCATAGATGTCCAAA
TAAGCACACCACGCCCAGTCAAATTACTTAGAGGCCATACTCTTGTCCTC
AATTGTACTGCTACCACTCCCTTGAACACGAGAGTTCAAATGACCTGGAG
TTACCCTGATGAAAAAAATAAGAGAGCTTCCGTAAGGCGACGAATTGACC
AAAGCAATTCCCATGCCAACATATTCTACAGTGTTCTTACTATTGACAAA
ATGCAGAACAAAGACAAAGGACTTTATACTTGTCGTGTAAGGAGTGGACC
ATCATTCAAATCTGTTAACACCTCAGTGCATATATATGATAAAGCATTCA
TCACTGTGAAACATCGAAAACAGCAGGTGCTTGAAACCGTAGCTGGCAAG
CGGTCTTACCGGCTCTCTATGAAAGTGAAGGCATTTCCCTCGCCGGAAGT
TGTATGGTTAAAAGATGGGTTACCTGCGACTGAGAAATCTGCTCGCTATT
TGACTCGTGGCTACTCGTTAATTATCAAGGACGTAACTGAAGAGGATGCA
GGGAATTATACAATCTTGCTGAGCATAAAACAGTCAAATGTGTTTAAAAA
CCTCACTGCCACTCTAATTGTCAATGTGAAACCCCAGATTTACGAAAAGG
CCGTGTCATCGTTTCCAGACCCGGCTCTCTACCCACTGGGCAGCAGACAA
ATCCTGACTTGTACCGCATATGGTATCCCTCAACCTACAATCAAGTGGTT
CTGGCACCCCTGTAACCATAATCATTCCGAAGCAAGGTGTGACTTTTGTT
CCAATAATGAAGAGTCCTTTATCCTGGATGCTGACAGCAACATGGGAAAC
AGAATTGAGAGCATCACTCAGCGCATGGCAATAATAGAAGGAAAGAATAA
GCTTCCACCAGCTAACAGTTCTTTCATGTTGCCACCTACAAGCTTCTCTT
CCAACTACTTCCATTTCCTTCCGTGA
Extracellular Region of Wildtype Human
VEGFR-1 (the seven Ig-like domains are
shown in bold and underlined)
(SEQ ID NO: 23)
sklk dpelslkgtq himgagqtlh lqcrgeaahk wslpemvske
serlsitksa cgrngkqfcs tltlntaqan htgfysckyl
avptskkket esaiyifisd tgrpfvemys eipeiihmte
grelvipery tspnitvtlk kfpldtlipd gkriiwdsrk
gfiisnatyk eiglltceat vnghlyktny lthrqtntii
dvqistprpv kllrghtivl nctattpint rvqmtwsypd
eknkrasvrr ridqsnshan ifysvltidk mqnkdkglyt
crvrsgpsfk svntsvhiyd kafitvkhrk qqvletvagk
rsyrlsmkvk afpspevvwl kdglpateks aryltrgysl
iikdvteeda gnytillsik qsnvfknita tlivnvkpqi
yekayssfpd palvplgsrq iltctaygip qptikwfwhp
cnhnhsearc dfcsnneesf ildadsnmgn riesitqrma
iiegknkmas tivvadsris giyiciasnk vgtvgrnisf
yitdvpngfh vnlekmpteg edlklsctvn kflyrdvtwi
llrtvnnrtm hysiskqkma itkehsitln ltimnvslqd
sgtyacrarn vytgeeilqk keitirdqea pyllrnlsdh
tvaisssttl dchangvpep qitwfknnhk iqqepqiilq
pgsstlfier vteedegvyh ckatnqkgsv essayltvqg
tsdksnle
Extracellular Region of Wildtype Mouse
VEGFR-1
(SEQ ID NO: 24)
ygsgsklk vpelslkgtq hvmqagqtlf lkcrgeaahs
wslpttvsqe dkrlsitpps acgrdnrqfc stltldtaqa
nhtglytcry 1ptstskkkk aessiyifvs dagspfiemh
tdipklvhmt egrqliiper vtspnvtvtl kkfpfdtltp
dgqritwdsr rgfiianaty keigllncea tvnghlyqtn
ylthrqtnti ldvqirppsp vrllhgqtiv lnctatteln
trvqmswnyp gkatkrasir qridrshshn nvfhsvlkin
nvesrdkgly tcrvksgssf qsfntsvhvy ekgfisvkhr
kqpvqettag rrsyrlsmkv kafpspeivw lkdgspatlk
sarylvhgys liikdvtted agdytillgi kqsrlfknit
atlivnvkpq iyeksysslp spplyplgsr qvltctvygi
prptitwlwh pchhnhsker ydfctenees fildpssnlg
nriesisqrm tviegtnktv stivvadsqt pgiyscrafn
kigtvernik fyvtdvpngf hvslekmpae gedlklscvv
nkflyrditw illrtvnnrt mhhsiskqkm attqdysitl
nlviknvsle dsgtyacrar niytgedilr ktevlvrdse
aphllqnlsd yevsisgstt ldcgargvpa pqitwfknnh
kiqqepgiil gpgnstlfie rvteedegvy rcratnqkga
vesaayltvq gtsdksnle
Extracellular Region of Wildtype Rat
VEGFR-1
(SEQ ID NO: 25)
ycsgsklk gpelslkgtq hvmqagqtlf lkcrgeaahs
wslpttvsqe dkklsvtrsa cgrnnrqfcs tltlnmaqan
htglyscryl pkstskekkm esaiyifvsd agspfiemhs
dipklvhmte greliipery tspnitvtlk kfpfdaltpd
gqriawdsrr gfiianatyk eiglltceat vnghlyqtsy
lthrqtntil dvqisppspv rflrgqtivl nctvttdlnt
rvqmswnypg katkrasirq ridqsnphsn vfhsvlkinn
vesrdkglyt crvksgssfr tfntsvhvye kgfisvkhrk
qqvqetiagk rshrlsmkvk afpspevvwl kdgvpateks
arysvhgysl iikdvtaeda gdytillgik qsklfrnita
tlivnvkpqi yeksysslps pplyplgsrq vltctvygip
qptikwlwhp chynhskern dfcfgseesf ildsssnign
riegitqrmm viegtnktvs tivvadsrtp gsysckafnk
igtverdirf yvtdvpngfh vslekipteg edlklscvvs
kflyrditwi llrtvnnrtm hhsiskqkma ttqdysitln
lviknvsled sgtyacrarn iytgeeilrk tevlvrdlea
plllqnlsdh evsisgsttl dcqargvpap qitwfknnhk
iqqepgiilg pgnstlfier vteedegvyr cratnqkgvv
essayltvqg tsdksnle
Extracellular Region of Wildtype Human
VEGFR-2 (the seven Ig-like domains are
shown in bold and underlined)
(SEQ ID NO: 26)
asvglpsysld lprlsiqkdi ltikanttlq itcrgqrdld
wlwpnngsgs eqrvevtecs dglfcktlti pkvigndtga
ykcfyretdl asviyvyvqd yrspfiasys dqhgvvyite
nknktvvipc lgsisnlnvs lcarypekrf vpdgnriswd
skkgftipsy misyagmvfc eakindesyq simyivvvvg
yriydvvlsp shgielsvge klvinctart elnvgidfnw
eypsskhqhk klvnrdlktq sgsemkkfls tltidgvtrs
dqglytcaas sglmtkknst fvrvhekpfv afgsgmeslv
eatvgervri pakylgyppp eikwykngip lesnhtikag
hvltimevse rdtgnytvil tnpiskekqs hvvslvvyvp
pqiqekslis pvdsvqygtt qtltctvyai ppphhihwyw
qleeecanep sqaysvtnpy pceewrsved fqggnkievn
knqfaliegk nktvstiviq aanvsalykc eavnkvgrge
rvisfhvtrg peitlqpdmq pteqesyslw ctadrstfen
ltwyklgpqp lpihvgelpt pvcknldtlw klnatmfsns
tndilimelk naslqdqgdy vclaqdrktk krhcvvrqlt
vlervaptit qnlengttsi gesievscta sgnpppqimw
fkdnetived sgivlkdgnr nltirrvrke deglytcqac
svlgcakvea ffiiegaqek tnle
Extracellular Region of Wildtype Mouse
VEGFR-2
(SEQ ID NO: 27)
asvglpgdflh ppklstqkdi ltilanttlq itcrgqrdld
wlwpnaqrds eervlvtecg ggdsifcktl tiprvvgndt
gaykcsyrdv diastvyvyv rdyrspfias vsdqhgivyi
tenknktvvi pergsisnln vslcarypek rfvpdgnris
wdseigftlp symisyagmv fceakindet yqsimyivvv
vgyriydvil sppheielsa geklvincta rtelnvgldf
twhsppsksh hkkivnrdvk pfpgtvakmf lstltiesvt
ksdqgeytcv assgrmikrn rtfvrvhtkp fiafgsgmks
lveatvgsqv ripvkylsyp apdikwyrng rpiesnytmi
vgdeltimev terdagnytv iltnpismek qshmvslvvn
vppqigekal ispmdsyqyg tmqtltctvy anpplhhiqw
ywqleeacsy rpgqtspyac kewrhvedfq ggnkievtkn
qyaliegknk tvstiviqaa nvsalykcea inkagrgery
isfhvirgpe itvqpaaqpt eqesysllct adrntfenit
wyklgsqats vhmgesltpv cknldalwkl ngtmfsnstn
dilivafqna slqdqgdyvc saqdkktkkr hclvkqliil
ermapmitgn lenqtttige tievtcpasg nptphitwfk
dnetivedsg ivirdgnrn1 tirrvrkedg glytcqacnv
lgcaraetlf iiegaqektn le
Extracellular Region of Wildtype
Rat VEGFR-2
(SEQ ID NO: 28)
asvglpgdslh ppklstqkdi ltilanttlq itcrgqrdld
wlwpntprds eervlvtecg dsifcktltv prvvgndtga
ykcfyrdtdv ssivyvyvqd hrspfiasys dehgivyite
nknktvvipc rgsisnlnvs lcarypekrf vpdgnriswd
sekgftipsy misyagmvfc eakindetyq simyivlvvg
yriydvvlsp pheielsage klvinctart elnvgldfsw
qfpsskhqhk kivnrdvksl pgtvakmfls tltidsvtks
dqgeytctay sglmtkknkt fvrvhtkpfi afgsgmkslv
eatvgsqvri pvkylsypap dikwyrngrp iesnytmivg
deltimevse rdagnytvil tnpismekqs hmvslvvnvp
pqigekalis pmdsyqygtm qtltctvyan pplhhiqwyw
qleeacsyrp sqtnpytcke wrhvkdfqgg nkievtknqy
aliegknktv stiviqaayv salykceain kagrgervis
fhvirgpeit vqpatqpter esmsllctad rntfenitwy
klgsqatsvh mgesltpvck nldalwklng tvfsnstndi
livafqnasl qdqgnyvcsa qdkktkkrhc lvkqlviler
mapmitgnle nqtttigeti evvcptsgnp tplitwfkdn
etivedsgiv lkdgnrniti rrvrkedggl ytcqacnvlg
caraetlfii egvqektnle
Extracellular Region of Wildtype
Human VEGFR-3 (the seven Ig-like
domains are shown in bold)
(SEQ ID NO: 29)
ysmtpp tlniteeshv idtgdslsis crgqhplewa
wpgaqeapat gdkdsedtgv vrdcegtdar pyckvlllhe
vhandtgsyv cyykyikari egttaassyv fvrdfeqpfi
nkpdtllvnr kdamwvpclv sipglnvtlr sgssvlwpdg
qevvwddrrg mlvstpllhd alylqcettw gdgdflsnpf
lvhitgnely diqllprksl ellvgeklvl nctvwaefns
gvtfdwdypg kqaergkwvp errsqqthte lssiltihnv
sqhdlgsyvc kanngiqrfr estevivhen pfisvewlkg
pileatagde lvklpvklaa vpppefgwyk dgkalsgrhs
phalvlkevt eastgtytla lwnsaaglrr nislelvvnv
ppqihekeas spsiysrhsr qaltctaygv plplsiqwhw
rpwtpckmfa qrslrrrqqq dlmpqcrdwr avttqdavnp
iesldtwtef vegknktvsk lvignanvsa mykcvvsnkv
gqderliyfy vttipdgfti eskpseelle ggpvllscqa
dsykyehlrw yrinlstlhd ahgnpllldc knvhlfatpl
aasleevapg arhatlslsi prvapehegh yvcevqdrrs
hdkhchkkyl svgaleaprl tqnitdllvn vsdslemgcl
vagahapsiv wykderllee ksgvdladsn qklsiqzvre
edagrylcsv cnakgcvnss asvavegsed kgsmeivilv
Extracellular Region of Wildtype Mouse
VEGFR-3
(SEQ ID NO: 30)
ysmtpp tlnitedsyv idtgdslsis crgqhplewt
wpgagevltt ggkdsedtry vhdcegtear pyckvillaq
thanntgsyh cyykyikari egttaastyv fvrdfkhpfi
nkpdtllvnr kdsmwvpclv sipglnitlr sqssalhpdg
qevlwddrrg mrvptqllrd alylqcettw gdqnflsnlf
vvhitgnely diglypkksm ellvgeklvl nctvwaefds
gvtfdwdypg kqaerakwvp errsqqthte lssiltihnv
sqndlgpyvc eanngiqrfr estevivhek pfisvewlkg
pvleatagde lvklpvklaa ypppefqwyk drkavtgrhn
phalvlkevt easagvytla lwnsaaglrq nislelvvnv
pphihekeas spsiysrhsr qtltctaygv pqplsvqwhw
rpwtpcktfa qrslrrrqqr dgmpqcrdwk evttqdavnp
iesldswtef vegknktvsk lviqdanvsa mykcvvvnkv
gqderliyfy vttipdgfsi esepsedple gqsvrlscra
dnytyehlrw yrinlstlhd aqgnpllldc knvhlfatpl
eanleeaepg arhatlslni prvapedegd yvcevqdrrs
qdkhchkkyl svgaleaprl tqnitdllvn vsdslemrcp
vagahvpsiv wykderllek esgidladsn qrlsiqrvre
edagrylcsv cnakgcvnss asvavegsed kgsme
Extracellular Region of Wildtype Rat
VEGFR-3
(SEQ ID NO: 31)
ysmtpp tlnitedsyv idtgdslsis crgqhplewt
wrgagevltt ggkdsedtqv vqdcegtear pyckvlslaq
thanntgsyy cyykyikari egttaastyv fvrdfeqpfi
nkpdtllvnr kdsmwvpclv sipglnitlr sqssvlhpdg
qevlwddrrg mrvpt111rd alylqcettw gdqdflsnpf
lvhitgnely diglypkks1 ellvgeklvl nctvwaefds
gvtfdwdypg kqaerakwvp errsqqthte lssiltihnv
sqhdlgpyvc eanngiqqfr estevivhek pfisvewlkg
pvleatagde mvklpvklaa ypppefqwyk drkavtgrhn
phalvlkevt easagvytla lwnsaaglrq nislelvvnv
pphihekeas spsiysrhsr qtltcttygv pqplsvqwhw
rpwtpcktfa qrslrrrqpr dgmpqcrdwk evttqdavnp
iesldtwtes vegknktvsk lviqdanvsa mykcvvfnkv
gqderliyfy vttipdgfsi esepsedple gqsvrlscra
dnytyehlrw yrinlstlhd aqgnpllldc knvhlfatpl
eanleeaepg arhatlslni prvapedegd yvcevqdrrs
qdkhchkkyl svgaleaprl tqnitdllvn vrtslemrcp
vagahvpsiv wykderllek esgidladsn qrlsiqrvre
edagrylcsv cnakgcvnss asvavegsed kgsme

In some examples, a soluble VEGF receptor can further include a stabilizing domain (e.g., a Fc domain or a portion of a Fc domain). For example, a stabilizing domain can be an IgG1 Fc domain (e.g., a human wildtype IgG1 Fc domain or a portion thereof). For example, a stabilizing domain can be an IgG2 Fc domain (e.g., a human wildtype IgG2 Fc domain or a portion thereof). For example, a stabilizing domain can be an IgG3 Fc domain (e.g., a human wildtype IgG3 domain or a portion thereof).

Non-limiting examples of human wildtype IgG1 Fc domain, human wildtype IgG2 Fc domain, and human wildtype IgG3 Fc domain are shown below.

Human Wildtype IgG1 FC Domain
(SEQ ID NO: 32)
pcpapellgg psvflfppkp kdtlmisrtp evtcvvvdvs
hedpevkfnw yvdgvevhna ktkpreeqyn styrvvsvlt
vlhqdwlngk eykckvsnka lpapiektis kakgqprepq
vytlppsrde ltknqvsltc lvkgfypsdi avewesngqp
ennykttppv ldsdgsffly skltvdksrw qqgnvfscsv
mhealhnhyt qkslslspgk
Human Wildtype IgG2 FC Region
(SEQ ID NO: 33)
vecppcpapp vagpsvflfp pkpkdtlmis rtpevtcvvv
dvshedpevq fnwyvdgvev hnaktkpree qfnstfrvvs
vltvvhqdwl ngkeykckvs nkglpapiek tisktkgqpr
epqvytlpps reemtknqvs ltclvkgfyp sdiavewesn
gqpennyktt ppmldsdgsf flyskltvdk srwqqgnvfs
csvmhealhn hytqkslsls pgk
Human Wildtype IgG3 FC Region
(SEQ ID NO: 34)
tcprcpapel lggpsvflfp pkpkdtlmis rtpevtcvvv
dvshedpevq fkwyvdgvev hnaktkpree qfnstfrvvs
vltvlhqdwl ngkeykckvs nkalpapiek tisktkgqpr
epqvytlpps reemtknqvs ltclvkgfyp sdiavewess
gqpennyktt ppmldsdgsf flyskltvdk srwqqgnifs
csvmhealhn rftqkslsls pgk

In some embodiments, the soluble VEGF receptor is aflibercept (Eylea®). Aflibercept includes portions of human VEGF receptors 1 and 2 extracellular domains fused to the Fc portion of human IgG1 (size ˜115 kDa). Aflibercept inhibits the activity of VEGF-A, VEGF-B, and PIGF. Aflibercept has a K_{D} for VEGF-A of 0.49 pM. See, e.g., WO 2017/218974.

Amino Encoding aflibercept
(SEQ ID NO: 12)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

In some embodiments of a soluble VEGF receptor includes a sequence that is at least 80% identical (e.g., at least 82%, at least 84%, at least 86%, at least 88%, at least 90%, at least 92%, at least 94%, at least 96%, at least 98%, or at least 99%) identical to SEQ ID NO: 12.

In some embodiments of the soluble VEGF receptor includes an extracellular domain that is or includes the sequence of SEQ ID NO: 12, except that it includes one, two, three, four, five, six, seven, eight, nine, ten, eleven, twelve, thirteen, fourteen, or fifteen amino acid substitutions in the sequence of SEQ ID NO: 12.

Additional examples of soluble VEGF receptors are described in, e.g., Kendall et al., PNAS 90: 10705-10709, 1993; Kendall et al., Biochem Biophys Res Commun 226: 324-328, 1996; Failla et al., Int J Mol Sci 19(5):pii. E1306, 2018; and Jung et al., PLoS One 7(9): e44572.

### Vectors

Recombinant AAV vectors or “rAAVs” are typically composed of, at a minimum, a transgene or a portion thereof and a regulatory sequence, and optionally 5′ and 3′ AAV inverted terminal repeats (ITRs). Such a recombinant AAV vector is packaged into a capsid and delivered to a selected target cell (e.g., a cochlear hair cell).

The AAV sequences of the vector typically comprise the cis-acting 5′ and 3′ ITR sequences (See, e.g., B. J. Carter, in “Handbook of Parvoviruses”, ed., P. Tijsser, CRC Press, pp. 155 168, 1990). Typical AAV ITR sequences are about 145 nucleotides in length. In some embodiments, at least 75% of a typical ITR sequence (e.g., at least 80%, at least 85%, at least 90%, or at least 95%) is incorporated into the AAV vector. The ability to modify these ITR sequences is within the skill of the art. (See, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996). In some embodiments, any of the coding sequences described herein is flanked by 5′ and 3′ AAV ITR sequences in the AAV vectors. The AAV ITR sequences may be obtained from any known AAV, including presently identified AAV types.

AAV vectors as described herein may include any of the regulatory elements described herein (e.g., one or more of a promoter, a polyadenylation (poly(A)) signal sequence, and an IRES).

In some embodiments, the vector(s) is an adenovirus (see, e.g., Dmitriev et al. (1998) J Virol. 72: 9706-9713; and Poulin et al., J Virol 8: 10074-10086, 2010). In some embodiments, the vector(s) is a retrovirus (see, e.g., Maier et al. (2010) Future Microbiol 5: 1507-23).

The vectors provided herein can be of different sizes. The choice of vector that is used in any of the compositions, kits, and methods described herein may depend on the size of the vector.

In some embodiments, the vector(s) can have a total number of nucleotides of up to 10 kb. In some embodiments, the viral vector(s) can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some embodiments, the vector(s) is a lentivirus and can have a total number of nucleotides of up to 8 kb. In some examples, the lentivirus(es) can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb.

In some embodiments, the vector(s) is an adenovirus and can have a total number of nucleotides of up to 8 kb. In some embodiments, the adenovirus(es) can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kh, about 6 kb to about 8 kb, or about 7 kb to about 8 kb.

In some embodiments, the vector(s) is an adeno-associated virus (AAV vector) and can include a total number of nucleotides of up to 5 kb. In some embodiments, the AAV vector(s) can include a total number of nucleotides in the range of about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

A variety of different methods known in the art can be used to introduce any of vectors disclosed herein into a mammalian cell (e.g., an inner ear cell, a cochlear inner hair cell). Non-limiting examples of methods for introducing nucleic acid into a mammalian cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection.

Any of the vectors described herein can further include a control sequence, e.g., a control sequence selected from the group of a transcription initiation sequence, a transcription termination sequence, a promoter sequence, an enhancer sequence, an RNA splicing sequence, a polyadenylation (polyA) signal, and a Kozak consensus sequence. Non-limiting examples of these control sequences are described herein. In some embodiments, a promoter can be a native promoter, a constitutive promoter, an inducible promoter, and/or a tissue-specific promoter.

### Promoters

The term “promoter” means a DNA sequence recognized by enzymes/proteins in a mammalian cell required to initiate the transcription of a specific gene. A promoter typically refers to, e.g., a nucleotide sequence to which an RNA polymerase and/or any associated factor binds and at which transcription is initiated. Non-limiting examples of promoters are described herein. Additional examples of promoters are known in the art.

In some embodiments, a vector (e.g., an adeno-associated virus (AAV) vector) encoding an antibody (e.g., an antibody that binds specifically to VEGF or an antigen-binding antibody fragment thereof,) can include a promoter and/or an enhancer. The vector encoding the antibody or antigen-binding antibody fragment can include any of the promoters and/or enhancers described herein or known in the art.

In some embodiments, the promoter is an inducible promoter, a constitutive promoter, a mammalian cell promoter, a viral promoter, a chimeric promoter, an engineered promoter, a tissue-specific promoter, or any other type of promoter known in the art. In some embodiments, the promoter is a RNA polymerase II promoter, such as a mammalian RNA polymerase II promoter. In some embodiments, the promoter is a RNA polymerase III promoter, including, but not limited to, a H1 promoter, a human U6 promoter, a mouse U6 promoter, or a swine U6 promoter. The promoter will generally be one that is able to promote transcription in an inner hair cell In some examples, the promoter is a cochlea-specific promoter or a cochlea-oriented promoter.

A variety of promoters are known in the art that can be used herein. Non-limiting examples of promoters that can be used herein include: human EF1a, human cytomegalovirus (CMV) (U.S. Pat. No. 5,168,062), human ubiquitin C (UBC), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g. human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2κ b, HSP70, proliferin, tumor necrosis factor, thyroid stimulating hormone α gene, immunoglobulin light chain, T-cell receptor, HLA DQα and DQβ, interleukin-2 receptor, MHC class II, MHC class II HLA-DRα, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, and Gibbon Ape Leukemia Virus (GALV) promoters. Additional examples of promoters are known in the art. See, e.g., Lodish, Molecular Cell Biology, Freeman and Company, New York 2007. In some embodiments, the promoter is the CMV immediate early promoter. In some embodiments, the promoter is a CAG promoter or a CAG/CBA promoter.

The term “constitutive” promoter refers to a nucleotide sequence that, when operably linked with a nucleic acid encoding a protein (e.g., an antibody or an antigen-binding antibody fragment), causes RNA to be transcribed from the nucleic acid in a mammalian cell under most or all physiological conditions.

Examples of constitutive promoters include, without limitation, the retroviral Rous sarcoma virus (RSV) LTR promoter, the cytomegalovirus (CMV) promoter (see, e.g., Boshart et al, Cell 41:521-530, 1985), the SV40 promoter, the dihydrofolate reductase promoter, the beta-actin promoter, the phosphoglycerol kinase (PGK) promoter, and the EF1-alpha promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Additional examples of inducible promoters are known in the art.

Examples of inducible promoters regulated by exogenously supplied compounds include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. U.S.A. 93:3346-3351, 1996), the tetracycline-repressible system (Gossen et al, Proc. Natl. Acad. Sci. U.S.A. 89:5547-5551, 1992), the tetracycline-inducible system (Gossen et al, Science 268:1766-1769, 1995, see also Harvey et al, Curr. Opin. Chem. Biol. 2:512-518, 1998), the RU486-inducible system (Wang et al, Nat. Biotech. 15:239-243, 1997) and Wang et al, Gene Ther. 4:432-441, 1997), and the rapamycin-inducible system (Magari et al. J. Clin. Invest. 100:2865-2872, 1997).

The term “tissue-specific” promoter refers to a promoter that is active only in certain specific cell types and/or tissues (e.g., transcription of a specific gene occurs only within cells expressing transcription regulatory proteins that bind to the tissue-specific promoter).

In some embodiments, the regulatory sequences impart tissue-specific gene expression capabilities. In some cases, the tissue-specific regulatory sequences bind tissue-specific transcription factors that induce transcription in a tissue-specific manner.

Exemplary tissue-specific promoters include but are not limited to the following: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, an alpha-myosin heavy chain (a-MHC) promoter, and a cardiac Troponin T (cTnT) promoter. Additional exemplary promoters include Beta-actin promoter, hepatitis B virus core promoter (Sandig et al., Gene Ther. 3:1002-1009, 1996), alpha-fetoprotein (AFP) promoter (Arbuthnot et al., Hum. Gene Ther. 7:1503-1514, 1996), bone osteocalcin promoter (Stein et al., Mol. Biol. Rep. 24:185-196, 1997); bone sialoprotein promoter (Chen et al., J. Bone Miner. Res. 11:654-664, 1996), CD2 promoter (Hansal et al., J. Immunol. 161:1063-1068, 1998); immunoglobulin heavy chain promoter; T cell receptor alpha-chain promoter, neuronal such as neuron-specific enolase (NSE) promoter (Andersen et al., Cell. Mol. Neurobiol. 13:503-515, 1993), neurofilament light-chain gene promoter (Piccioli et al., Proc. Natl. Acad. Sci. U.S.A. 88:5611-5615, 1991), and the neuron-specific vgf gene promoter (Piccioli et al., Neuron 15:373-384, 1995).

In some embodiments, the tissue-specific promoter is a cochlea-specific promoter. In some embodiments, the tissue-specific promoter is a cochlear hair cell-specific promoter. Non-limiting examples of cochlear hair cell-specific promoters include but are not limited to: a ATOH1 promoter, a POU4F3 promoter, a LHX3 promoter, a MYO7A promoter, a MYO6 promoter, a α9ACHR promoter, and a α10ACHR promoter. In some embodiments, the promoter is an cochlear hair cell-specific promoter such as a PRESTIN promoter or an ONCOMOD promoter. See, e.g., Zheng et al., Nature 405:149-155, 2000; Tian et al. Dev. Dyn. 231:199-203, 2004; and Ryan et al., Adv. Otorhinolaryngol. 66: 99-115, 2009.

### Enhancers

In some instances, a vector (e.g., an AAV vector) can include an enhancer sequence. The term “enhancer” refers to a nucleotide sequence that can increase the level of transcription of a nucleic acid encoding a protein of interest (e.g., an antibody that binds specifically to VEGF or an antigen-binding antibody fragment thereof, or a soluble VEGF receptor). Enhancer sequences (50-1500 basepairs in length) generally increase the level of transcription by providing additional binding sites for transcription-associated proteins (e.g., transcription factors). In some embodiments, an enhancer sequence is found within an intronic sequence. Unlike promoter sequences, enhancer sequences can act at much larger distance away from the transcription start site (e.g., as compared to a promoter). Non-limiting examples of enhancers include a RSV enhancer, a CMV enhancer, and a SV40 enhancer.

### Poly(A) Signal Sequence

In some embodiments, any of the vectors provided herein (e.g., an AAV vector) can include a polyadenylation (poly(A)) signal sequence. Most nascent eukaryotic mRNAs possess a poly(A) tail at their 3′ end which is added during a complex process that includes cleavage of the primary transcript and a coupled polyadenylation reaction driven by the poly(A) signal sequence (see, e.g., Proudfoot et al., Cell 108:501-512, 2002). The poly(A) tail confers mRNA stability and transferability (Molecular Biology of the Cell, Third Edition by B. Alberts et al., Garland Publishing, 1994). In some embodiments, the poly(A) signal sequence is positioned 3′ to the nucleic acid sequence encoding the antibody heavy chain, the antibody light chain, the antigen-binding antibody fragment, or the soluble VEGF receptor.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In higher eukaryotes, the poly(A) tail is added onto transcripts that contain a specific sequence, the polyadenylation (or poly(A)) signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but also can occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used, including those derived from bovine growth hormone (bgh) (Woychik et al., Proc. Natl. Acad. Sci. U.S.A. 81(13):3944-3948, 1984; U.S. Pat. No. 5,122,458), mouse-β-globin, mouse-α-globin (Orkin et al., EMBO J. 4(2):453-456, 1985; Thein et al., Blood 71(2):313-319, 1988), human collagen, polyoma virus (Batt et al., Mol. Cell Biol. 15(9):4783- 4790, 1995), the Herpes simplex virus thymidine kinase gene (HSV TK), IgG heavy-chain gene polyadenylation signal (U.S. 2006/0040354), human growth hormone (hGH) (Szymanski et al., Mol. Therapy 15(7):1340-1347, 2007), the group consisting of SV40 poly(A) site, such as the SV40 late and early poly(A) site (Schek et al., Mol. Cell Biol. 12(12):5386-5393, 1992).

The poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414).

In some embodiments, the poly(A) signal sequence can be a synthetic polyadenylation site (see, e.g., the pCl-neo expression vector of Promega that is based on Levitt el al, Genes Dev. 3(7):1019-1025, 1989). In some embodiments, the poly(A) signal sequence is the polyadenylation signal of soluble neuropilin-1 (sNRP) (AAATAAAATACGAAATG; SEQ ID NO: 11) (see, e.g., WO 05/073384). Additional examples of poly(A) signal sequences are known in the art.

### Internal Ribosome Entry Site (IRES)

In some embodiments, a vector (e.g., an adeno-associated virus (AAV) vector) encoding an antibody (e.g., an antibody heavy chain and an antibody light chain), an antigen-binding antibody fragment, or a soluble VEGF receptor can include a polynucleotide internal ribosome entry site (IRES). An IRES sequence is used to produce more than one polypeptide from a single gene transcript. An IRES forms a complex secondary structure that allows translation initiation to occur from any position with an mRNA immediately downstream from where the IRES is located (see, e.g., Pelletier and Sonenberg, Mol. Cell. Biol. 8(3):1103-1112, 1988).

There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV). See e.g., Alberts, Molecular Biology of the Cell, Garland Science, 2002; and Hellen et al., Genes Dev. 15(13):1593-612, 2001.

In some embodiments, the IRES sequence that is incorporated into the AAV vector is the foot and mouth disease virus (FMDV) 2A sequence. The Foot and Mouth Disease Virus 2A sequence is a small peptide (approximately 18 amino acids in length) that has been shown to mediate the cleavage of polyproteins (Ryan, M D et al., EMBO 4:928-933, 1994; Mattion et al., J. Virology 70:8124-8127, 1996; Furler et al., Gene Therapy 8:864-873, 2001; and Halpin et al., Plant Journal 4:453-459, 1999). The cleavage activity of the 2A sequence has previously been demonstrated in artificial systems including plasmids and gene therapy vectors (AAV and retroviruses) (Ryan et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999; de Felipe et al., Gene Therapy 6:198-208, 1999; de Felipe et al., Human Gene Therapy 11:1921-1931, 2000; and Klump et al., Gene Therapy 8:811-817, 2001).

### Reporter Sequences

Any of the AAVs provided herein can optionally include a sequence encoding a reporter protein (“a reporter sequence”). Non-limiting examples of reporter sequences include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. When associated with regulatory elements which drive their expression, the reporter sequence can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some embodiments, the reporter sequence is the LacZ gene, and the presence of a vector carrying the LacZ gene in a mammalian cell (e.g., a cochlear hair cell) is detected by assays for beta-galactosidase activity. When the reporter is a fluorescent protein (e.g., green fluorescent protein) or luciferase, the presence of a vector carrying the fluorescent protein or luciferase in a mammalian cell (e.g., a cochlear hair cell) may be measured by fluorescent techniques (e.g., fluorescent microscopy or FACS) or light production in a luminometer (e.g., a spectrophotometer or an IVIS imaging instrument). In some embodiments, the reporter sequence can be used to verify the tissue-specific targeting capabilities and tissue-specific promoter regulatory activity of any of the vectors described herein.

### Flanking Regions Untranslated Regions (UTRs)

In some embodiments, any of the adeno-associated virus (AAV) vectors can include an untranslated region, such as a 5′ UTR or a 3′ UTR.

Untranslated regions (UTRs) of a gene are transcribed but not translated. The 5′ UTR starts at the transcription start site and continues to the start codon but does not include the start codon. The 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into any of the vectors, compositions, kits, or methods as described herein to enhance the expression of an antibody (e.g., an antibody that binds specifically to VEGF), an antigen-binding antibody fragment (e.g., an antigen-binding fragment that binds specifically to VEGF), or a soluble VEGF receptor.

Natural 5′ UTRs include a sequence that plays a role in translation initiation. They harbor signatures like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. The 5′ UTRs have also been known to form secondary structures that are involved in elongation factor binding.

In some embodiments, a 5′ UTR is included in any of the vectors described herein. Non-limiting examples of 5′ UTRs, including those from the following genes: albumin, serum amyloid A, Apolipoprotein A/B/E, transferrin, alpha fetoprotein, erythropoietin, and Factor VIII, can be used to enhance expression of a nucleic acid molecule, such as a mRNA.

In some embodiments, a 5′ UTR from a mRNA that is transcribed by a cell in the cochlea can be included in any of the vectors, compositions, kits, and methods described herein.

3′ UTRs are known to have stretches of adenosines and uridines (in the RNA form) or thymidines (in the DNA form) embedded in them. These AU-rich signatures are particularly prevalent in genes with high rates of turnover. Based on their sequence features and functional properties, the AU-rich elements (AREs) can be separated into three classes (Chen et al., Mol. Cell. Biol. 15:5777-5788, 1995; Chen et al., Mol. Cell Biol. 15:2010-2018, 1995): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif. Two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

In some embodiments, the introduction, removal, or modification of 3′ UTR AREs can be used to modulate the stability of an mRNA encoding a protein of interest (e.g., any antibody described herein, any antigen-binding antibody fragment described herein, or any soluble VEGF receptor described herein). In other embodiments, AREs can be removed or mutated to increase the intracellular stability and thus increase translation and production of a protein of interest (e.g., any antibody described herein, any antigen-binding antibody fragment described herein, or any soluble VEGF receptor described herein).

In other embodiments, non-ARE sequences may be incorporated into the 5′ or 3′ UTRs. In some embodiments, introns or portions of intron sequences may be incorporated into the flanking regions of the polynucleotides in any of the vectors, compositions, kits, and methods provided herein. Incorporation of intronic sequences may increase protein production as well as mRNA levels.

Fc Mutations that Decrease the Half-Life of an Antibody, Antigen-Binding Antibody Fragment, or a Soluble VEGF Receptor in a Mammal

Any of the antibodies, antigen-binding antibody fragments, or soluble VEGF receptors described herein can include one or more (e.g., two, three, four, five, six, seven, eight, nine, or ten) amino acid substitutions in the Fc region that decrease the half-life of the antibody, the antigen-binding antibody fragment, or soluble VEGF receptor in a mammal, e.g., as compared to the half-life of an otherwise identical antibody, antigen-binding antibody fragment, or soluble VEGF receptor not including at least one of the one or more amino acid substitutions in the Fc region. Methods for determining the half-life of an antibody, antigen-binding antibody fragment, or soluble VEGF receptor in a mammal are well-known in the art.

Non-limiting examples of point mutations in a Fc mutation that can decrease the half-life of an antibody, an antigen-binding antibody fragment, or soluble VEGF receptor are described in Leabman et al., MAbs 5(6):896-903, 2013.

### Methods

Also provided herein are methods that include introducing into an inner ear of a mammal a therapeutically effective amount of an adeno-associated virus (AAV) vector that includes a nucleotide sequence encoding (a) a polypeptide including an antibody heavy chain variable domain (e.g., any of the exemplary antibody heavy chain variable domains described herein) operably linked to a signal peptide (e.g., any of the signal peptides described herein) and a polypeptide including an antibody light chain variable domain (e.g., any of the exemplary antibody light chain variable domains described herein) operably linked to a signal peptide (e.g., any of the signal peptides described herein); (b) a polypeptide including an antigen-binding antibody fragment (e.g., a scFv) (e.g., any of the exemplary antigen-binding antibody fragments described herein) operably linked to a signal peptide (e.g., any of the signal peptides described herein), or (c) a soluble VEGF receptor (e.g., any of the soluble VEGF receptors described herein) operably linked to a signal peptide (e.g., any of the signal peptides described herein). Also provided herein are methods for increasing the level of an antibody or an antigen-binding antibody fragment in an inner ear of a mammal in need thereof, that include: introducing into the inner ear of the mammal a therapeutically effective amount of an AAV vector that includes a nucleotide sequence encoding (a) a polypeptide including an antibody heavy chain variable domain (e.g., any of the antibody heavy chain variable domains described herein) operably linked to a signal peptide and a polypeptide including an antibody light chain variable domain (e.g., any of the antibody light chain variable domains described herein) operably linked to a signal peptide; or (b) a polypeptide including an antigen-binding antibody fragment (e.g., a scFv) (e.g., any of the exemplary antigen-binding antibody fragments described herein) operably linked to a signal peptide (e.g., any of the exemplary signal peptides described herein); where the introducing results in an increase (e.g., a 1% to 400% increase (or any of the subranges of this range described herein), or at least a 1%, at least a 10%, at least a 20%, at least a 30%, at least a 40%, at least a 50%, at least a 60%, at least a 70%, at least a 80%, at least a 90%, at least a 100%, at least a 150%, at least a 200%, at least a 250%, at least a 300%, at least a 350%, at least a 400%, at least a 450%, at least a 500%, at least a 550%, at least a 600%, at least a 650%, at least a 700% , at least a 750%, at least a 800%, at least a 850%, at least a 900%, at least a 950%, at least a 1000%, at least a 1100%, at least a 1200%, at least a 1300%, at least a 1400%, at least a 1500%, at least a 1600%, at least a 1700%, at least a 1800%, at least a 1900%, or at least a 2000% increase) in the level of the antibody or the antigen-binding antibody fragment in the inner ear of the mammal, e.g., as compared to the level of the antibody or the antigen-binding antibody fragment in the inner ear of the mammal prior to the administration.

Also provided herein are methods for increasing the level of a soluble VEGF receptor that include: introducing into the inner ear of the mammal a therapeutically effective amount of an AAV vector that includes a nucleotide sequence encoding a soluble VEGF receptor (e.g., any of the soluble VEGF receptors described herein) operably linked to a signal peptide (e.g., any of the signal peptides described herein);

where the introducing results in an increase (e.g., a 1% to 400% increase (or any of the subranges of this range described herein), or at least a 1%, at least a 10%, at least a 20%, at least a 30%, at least a 40%, at least a 50%, at least a 60%, at least a 70%, at least a 80%, at least a 90%, at least a 100%, at least a 150%, at least a 200%, at least a 250%, at least a 300%, at least a 350%, at least a 400%, at least a 450%, at least a 500%, at least a 550%, at least a 600%, at least a 650%, at least a 700% , at least a 750%, at least a 800%, at least a 850%, at least a 900%, at least a 950%, at least a 1000%, at least a 1100%, at least a 1200%, at least a 1300%, at least a 1400%, at least a 1500%, at least a 1600%, at least a 1700%, at least a 1800%, at least a 1900%, or at least a 2000% increase) in the level of the soluble VEGF receptor in the inner ear of the mammal, e.g., as compared to the level of the soluble VEGF receptor in the inner ear of the mammal prior to the administration.

Also provided herein are methods for treating an inner ear disorder in a mammal in need thereof that include introducing into the inner ear of the mammal a therapeutically effective amount of an AAV vector that comprises a nucleotide sequence encoding: (a) a polypeptide including an antibody heavy chain variable domain (e.g., e.g., any of the antibody heavy chain variable domains described herein) operably linked to a signal peptide (e.g., any of the signal peptides described herein) and a polypeptide comprising an antibody light chain variable domain (e.g., any of the antibody light chain variable domains described herein) operably linked to a signal peptide (e.g., any of the signal peptides described herein); (b) a polypeptide including an antigen-binding antibody fragment (e.g., any of the exemplary antigen-binding antibody fragments described herein) linked to a signal peptide (e.g., any of the signal peptides described herein); or (c) a soluble VEGF receptor (e.g., any of the soluble VEGR receptors described herein) operably linked to a signal peptide (e.g., any of the signal peptides described herein), where the introducing results in the treatment of the inner ear disorder in the mammal. In some embodiments, treatment of an inner ear disorder results in a reduction (e.g., a 1% to 100% reduction, or any of the subranges of this range described herein) in the severity, frequency, or number of symptoms of an inner ear disorder in a mammal following the introducing as compared to before the introducing. In some embodiments, treatment of any inner ear disorder results in an increase (e.g., a 1% to 400% increase, or any of the subranges of this range described herein) in the hearing (e.g., one or more metrics of hearing) of the mammal following the introducing as compared to before the introducing.

In some embodiments of any of these methods, the antibody or the antigen-binding antibody fragment, or the soluble VEGF receptor, binds specifically to a vascular endothelial growth factor (VEGF) (e.g., one of more of VEGF-A, VEGF-B, VEGF-C, and VEGF-D, e.g., one or more of human VEGF-A, human VEGF-B, human VEGF-C, and human VEGF-D). In some embodiments of any of these methods, the AAV vector further includes one or both of a promoter and a Kozak sequence that are operably linked to the sequence encoding the antibody or the antigen-binding antibody fragment. In some embodiments wherein the AAV vector comprises a promoter selected from the group consisting of: an inducible promoter, a constitutive promoter, and a tissue-specific promoter. In some embodiments of any of these methods, the AAV vector further includes a polyadenylation signal sequence. In some embodiments of any of these methods, the mammal is a human. In some embodiments of any of these methods, the mammal (e.g., the human) has been identified as having an inner ear disorder. In some embodiments of any of these methods, the mammal (e.g., the human) has previously been diagnosed as having an inner ear disorder. In some embodiments of any of these methods, the vector includes a nucleic acid sequence encoding a polypeptide comprising an antibody heavy chain and an antibody light chain. In some embodiments of any of these methods, the vector includes a nucleic acid sequence encoding an antigen-binding antibody fragment. In some embodiments of any of these methods, the vector include a nucleic acid sequence encoding a soluble VEGF receptor operably linked to a signal peptide.

Also provided herein are methods of reducing a VEGF activity (e.g., one or more of VEGF-A, VEGF-B, VEGF-C, and VEGF-D, e.g., one or more of human VEGF-A, human VEGF-B, human VEGF-C, and human VEGF-D) in an inner ear of a mammal in need thereof that include: introducing into the inner ear of the mammal a therapeutically effective amount of an AAV vector that includes a nucleotide sequence encoding (a) a polypeptide including an antibody heavy chain variable domain (e.g., any of the antibody heavy chain variable domains described herein) operably linked to a signal peptide (e.g., any of the signal peptides described herein) and a polypeptide including an antibody light chain variable domain (e.g., any of the antibody light chain variable domains described herein) operably linked to a signal peptide (e.g., any of the signal peptides described herein); (b) a polypeptide including an antigen-binding antibody fragment (e.g., a Fab or a scFv) (e.g., any of the antigen-binding antibody fragments described herein) operably linked to a signal peptide (e.g., any of the signal peptides described herein); or (c) a soluble VEGF receptor (e.g., any of the soluble VEGF receptors described herein) operably linked to a signal peptide (e.g., any of the signal peptides described herein), where the polypeptide of (a) includes an antibody that binds specifically to a VEGF and reduces a VEGF activity, the polypeptide of (b) includes an antigen-binding antibody fragment that binds specifically to a VEGF and reduces a VEGF activity, or the soluble VEGF receptor of (c) binds specifically to one or more VEGF proteins and reduces the activity of the one or more VEGF proteins; and where the introducing results in a reduction (e.g., a 1% to 100% reduction, or any of the subranges of this range described herein) in a VEGF activity (e.g., an activity of one or more of VEGF-A, VEGF-B, VEGF-C, and VEGF-D, e.g., one or more human VEGF-A, human VEGF-B, human VEGF-C, and human VEGF-D) in the inner ear of the mammal, e.g., as compared to the VEGF activity in the mammal prior to the introducing. A reduction in a VEGF activity in a mammal can be detected using any of the exemplary methods described herein.

Also provided herein are methods of treating acoustic neuroma, vestibular schwannoma, or neurofibromatosis type II (NF2) in an inner ear of a mammal that include: introducing into the inner ear of the mammal a therapeutically effective amount of an AAV vector that includes a nucleotide sequence encoding (a) a polypeptide including an antibody heavy chain variable domain (e.g., any of the antibody heavy chain variable domains described herein) operably linked to a signal peptide (e.g., any of the signal peptides described herein) and a polypeptide including an antibody light chain variable domain (e.g., any of the antibody light chain variable domains described herein) operably linked to a signal peptide (e.g., any of the signal peptides described herein); (b) a polypeptide including an antigen-binding antibody fragment (e.g., a Fab or a scFv) (e.g., any of the antigen-binding antibody fragments described herein) operably linked to a signal peptide (e.g., any of the signal peptides described herein), or (c) a soluble VEGF receptor (e.g., any of the soluble VEGF receptors described herein) operably linked to a signal peptide (e.g., any of the signal peptides described herein); where the polypeptide of (a) encodes an antibody that binds specifically to a VEGF (e.g., one or more of VEGF-A, VEGF-B, VEGF-C, and VEGF-D, e.g., one or more of human VEGF-A, human VEGF-B, human VEGF-C, and human VEGF-D) and reduces the VEGF activity, the polypeptide of (b) encodes an antigen-binding antibody fragment that binds specifically to a VEGF (e.g., one or more of VEGF-A, VEGF-B, VEGF-C, and VEGF-D, e.g., one or more of human VEGF-A, human VEGF-B, human VEGF-C, and human VEGF-D) and reduces the VEGF activity, or the soluble VEGF receptor of (c) binds to specifically to one or more of VEGF-A, VEGF-B, VEGF-C, and VEGF-D (e.g., one or more of human VEGF-A, human VEGF-B, human VEGF-C, and human VEGF-D) and where the introducing results in treatment of acoustic neuroma, vestibular schwannoma, or neurofibromatosis type II (NF2) in the inner ear of the mammal. As described herein, successful treatment of one or more of an acoustic neuroma, vestibular schwannoma, or neurofibromatosis type II can be detected by observing a reduction (e.g., a 1% to 100% decrease, or any of the subranges of this range described herein) in the number, severity, or frequency of one or more symptoms of an acoustic neuroma, vestibular schwannoma, or neurofibromatosis type II, respectively, in the mammal, e.g., as compared to before the introducing step.

In some embodiments of any of these methods, the vector includes a nucleic acid sequence encoding a polypeptide encoding an antibody heavy chain variable domain (e.g., any of the antibody heavy chains described herein) and an antibody light chain variable domain (e.g., any of the antibody light chain variable domains described herein). In some embodiments of any of these methods, the vector includes a nucleic acid sequence encoding a polypeptide comprising an antigen-binding antibody fragment (e.g., any of the antigen-binding antibody fragments described herein). In some embodiments of any of these methods, the vector includes a nucleic acid sequence encoding a soluble VEGF receptor (e.g., any of the soluble VEGF receptors described herein) operably linked to a signal peptide (e.g., any of the signal peptides described herein). In some embodiments of any of these methods, the AAV vector further includes one or both of a promoter and a Kozak sequence that are operably linked to the sequence encoding the antibody or the antigen-binding antibody fragment. In some embodiments, the AAV vector comprises a promoter, where the promoter is selected from the group consisting of: an inducible promoter, a constitutive promoter, or a tissue-specific promoter. In some embodiments, the AAV vector further includes a polyadenylation signal sequence. In some embodiments of any of these methods, the mammal is a human. In some embodiments of any of these methods, the mammal (e.g., the human) has been identified as having an inner ear disorder. In some embodiments of any of these methods, the mammal (e.g., the human) has previously been diagnosed as having an inner ear disorder. In some embodiments of any of these methods, the mammal (e.g., the human) has been identified or diagnosed as having drug-induced hearing loss. In some embodiments of any of these methods, the mammal (e.g., the human) has been identified or diagnosed as having age-related hearing loss.

In some embodiments, the antibody or antigen-binding fragment thereof includes a Fc region that includes one or more point mutations that decrease the half-life of the antibody or antigen-binding antibody fragment in vivo.

In some embodiments of any of these methods, two or more doses of any of the adeno-associated virus (AAV) vectors described herein are introduced or administered into the inner ear of the mammal or subject. Some embodiments of any of these methods can include introducing or administering a first dose of the adeno-associated virus (AAV) vectors into the inner ear of the mammal or subject, assessing hearing function of the mammal or subject following the introducing or the administering of the first dose, and administering an additional dose of the adeno-associated virus (AAV) vector into the inner ear of the mammal or subject found not to have a hearing function within a normal range (e.g., as determined using any test for hearing known in the art).

In some embodiments of any of the methods described herein, the adeno-associated virus (AAV) vectors can be formulated for intra-cochlear administration. In some embodiments of any of the methods described herein, the adeno-associated virus (AAV) vectors described herein can be administered via intra-cochlear administration or local administration. In some embodiments of any of the methods described herein, the adeno-associated virus (AAV) vectors are administered through the use of a medical device (e.g., any of the exemplary medical devices described herein).

In some embodiments, intra-cochlear administration can be performed using any of the methods described herein or known in the art. For example, an adeno-associated virus (AAV) vector can be administered or introduced into the cochlea using the following surgical technique: first using visualization with a 0 degree, 2.5-mm rigid endoscope, the external auditory canal is cleared and a round knife is used to sharply delineate an approximately 5-mm tympanomeatal flap. The tympanomeatal flap is then elevated and the middle ear is entered posteriorly. The chorda tympani nerve is identified and divided, and a currette is used to remove the scutal bone, exposing the round window membrane. To enhance apical distribution of the administered or introduced adeno-associated virus (AAV) vector, a surgical laser may be used to make a small 2-mm fenestration in the oval window to allow for perilymph displacement during trans-round window membrane infusion of the adeno-associated virus (AAV) vectors. The microinfusion device is then primed and brought into the surgical field. The device is maneuvered to the round window, and the tip is seated within the bony round window overhang to allow for penetration of the membrane by the microneedle(s). The footpedal is engaged to allow for a measured, steady infusion of the adeno-associated virus (AAV) vectors. The device is then withdrawn and the round window and stapes foot plate are sealed with a gelfoam patch.

In some embodiments of any of the methods described herein, the subject or mammal is a rodent, a non-human primate, or a human. In some embodiments of any of the methods described herein, the subject or mammal is an adult, a teenager, a juvenile, a child, a toddler, an infant, or a newborn. In some embodiments of any of the methods described herein, the subject or mammal is 1-5, 1-10, 1-20, 1-30, 1-40, 1-50, 1-60, 1-70, 1-80, 1-90, 1-100, 1-110, 2-5, 2-10, 10-20, 20-30, 30-40, 40-50, 50-60, 60-70, 70-80, 80-90, 90-100, 100-110, 10-30, 10-40, 10-50, 10-60, 10-70, 10-80, 10-90, 10-100, 10-110, 20-40, 20-50, 20-60, 20-70, 20-80, 20-90, 20-100, 20-110, 30-50, 30-60, 30-70, 30-80, 30-90, 30-100, 40-60, 40-70, 40-80, 40-90, 40-100, 50-70, 50-80, 50-90, 50-100, 60-80, 60-90, 60-100, 70-90, 70-100, 70-110, 80-100, 80-110, or 90-110 years of age. In some embodiments of any of the methods described herein, the subject or mammal is 1, 2, 3, 4, 5, 6, 7, 8, 9, 10, or 11 months of age.

In some embodiments of any of the methods described herein, the subject or mammal has or is at risk of developing hearing loss (e.g., drug-induced hearing loss). In some embodiments of any of the methods described herein, the subject or mammal has been previously identified as having a mutation in a VEGF gene.

In some embodiments, successful treatment of hearing loss (e.g., drug-induced hearing loss) can be determined in a subject using any of the conventional functional hearing tests known in the art. Non-limiting examples of functional hearing tests are various types of audiometric assays (e.g., pure-tone testing, speech testing, test of the middle ear, auditory brainstem response, and otoacoustic emissions).

Methods for introducing any of the adeno-associated virus (AAV) vectors described herein into a mammalian cell are known in the art (e.g., via lipofection or through the use of a viral vector, e.g., any of the viral vectors described herein).

### Pharmaceutical Compositions and Kits

In some embodiments, any of the compositions described herein can further include one or more agents that promote the entry of a nucleic acid or any of the vectors described herein into a mammalian cell (e.g., a liposome or cationic lipid). In some embodiments, any of the vectors described herein can be formulated using natural and/or synthetic polymers. Non-limiting examples of polymers that may be included in any of the compositions described herein can include, but are not limited to, DYNAMIC POLYCONJUGATE® (Arrowhead Research Corp., Pasadena, Calif), formulations from Mirus Bio (Madison, Wis.) and Roche Madison (Madison, Wis.), PhaseRX polymer formulations such as, without limitation, SMARTT POLYMER TECHNOLOGY® (PhaseRX, Seattle, Wash.), DMRI/DOPE, poloxamer, VAXFECTIN® adjuvant from Vical (San Diego, Calif), chitosan, cyclodextrin from Calando Pharmaceuticals (Pasadena, Calif), dendrimers and poly (lactic-co-glycolic acid) (PLGA) polymers, RONDEL™ (RNAi/Oligonucleotide Nanoparticle Delivery) polymers (Arrowhead Research Corporation, Pasadena, Calif.), and pH responsive co-block polymers, such as, but not limited to, those produced by PhaseRX (Seattle, Wash.). Many of these polymers have demonstrated efficacy in delivering oligonucleotides in vivo into a mammalian cell (see, e.g., deFougerolles, Human Gene Ther. 19:125-132, 2008; Rozema et al., Proc. Natl. Acad. Sci. U.S.A. 104:12982-12887, 2007; Rozema et al., Proc. Natl. Acad. Sci. U.S.A. 104:12982-12887, 2007; Hu-Lieskovan et al., Cancer Res. 65:8984-8982, 2005; Heidel et al., Proc. Natl. Acad. Sci. U.S.A. 104:5715-5721, 2007).

Any of the compositions described herein can be, e.g., a pharmaceutical composition. A pharmaceutical composition can include any of the compositions described herein and one or more pharmaceutically or physiologically acceptable carriers, diluents, or excipients. Such compositions may comprise one or more buffers, such as neutral-buffered saline, phosphate-buffered saline, and the like; one or more carbohydrates, such as glucose, mannose, sucrose, and dextran; mannitol; one or more proteins, polypeptides, or amino acids, such as glycine; one or more antioxidants; one or more chelating agents, such as EDTA or glutathione; and/or one or more preservatives.

In some embodiments, the composition includes a pharmaceutically acceptable carrier (e.g., phosphate buffered saline, saline, or bacteriostatic water). Upon formulation, solutions will be administered in a manner compatible with the dosage formulation and in such amount as is therapeutically effective. The formulations are easily administered in a variety of dosage forms such as injectable solutions, injectable gels, drug-release capsules, and the like.

As used herein, the term “pharmaceutically acceptable carrier” includes solvents, dispersion media, coatings, antibacterial agents, antifungal agents, and the like that are compatible with pharmaceutical administration. Supplementary active compounds can also be incorporated into any of the compostions described herein.

In some embodiments, a single dose of any of the compositions described herein can include a total sum amount of the at least two different vectors of at least 1 ng, at least 2 ng, at least 4 ng, about 6 ng, about 8 ng, at least 10 ng, at least 20 ng, at least 30 ng, at least 40 ng, at least 50 ng, at least 60 ng, at least 70 ng, at least 80 ng, at least 90 ng, at least 100 ng, at least 200 ng, at least 300 ng, at least 400 ng, at least 500 ng, at least 1 μg, at least 2 μg, at least 4 μg, at least 6 μg, at least 8 μg, at least 10 μg, at least 12 μg, at least 14 μg, at least 16 μg, at least 18 μg, at least 20 μg, at least 22 μg, at least 24 μg, at least 26 μg, at least 28 μg, at least 30 μg at least 32 μg, at least 34 μg, at least 36 μg, at least 38 μg, at least 40 μg, at least 42 μg, at least 44 μg, at least 46 μg, at least 48 μg, at least 50 μg, at least 52 μg, at least 54 μg, at least 56 μg, at least 58 μg, at least 60 μg, at least 62 μg, at least 64 μg, at least 66 μg, at least 68 μg, at least 70 μg, at least 72 μg, at least 74 μg, at least 76 μg, at least 78 μg, at least 80 μg, at least 82 μg, at least 84 μg, at least 86 μg, at least 88 μg, at least 90 μg, at least 92 μg, at least 94 μg, at least 96 μg, at least 98 μg, at least 100 μg, at least 102 μg, at least 104 μg, at least 106 μg, at least 108 μg, at least 110 μg, at least 112 μg, at least 114 μg, at least 116 μg, at least 118 μg, at least 120 μg, at least 122 μg, at least 124 μg, at least 126 μg, at least 128 μg, at least 130 μg at least 132 μg, at least 134 μg, at least 136 μg, at least 138 μg, at least 140 μg, at least 142 μg, at least 144 μg, at least 146 μg, at least 148 μg, at least 150 μg, at least 152 μg, at least 154 μg, at least 156 μg, at least 158 μg, at least 160 μg, at least 162 μg, at least 164 μg, at least 166 μg, at least 168 μg, at least 170 μg, at least 172 μg, at least 174 μg, at least 176 μg, at least 178 μg, at least 180 μg, at least 182 μg, at least 184 μg, at least 186 μg, at least 188 μg, at least 190 μg, at least 192 μg, at least 194 μg, at least 196 μg, at least 198 μg, or at least 200 μg, e.g., in a buffered solution.

The compositions provided herein can be, e.g., formulated to be compatible with their intended route of administration. A non-limiting example of an intended route of administration is local administration (e.g., intra-cochlear administration). In some embodiments, the therapeutic compositions are formulated to include a lipid nanoparticle. In some embodiments, the therapeutic compositions are formulated to include a polymeric nanoparticle. In some embodiments, the therapeutic compositions are formulated to comprise a mini-circle DNA. In some embodiments, the therapeutic compositions are formulated to comprise a CELiD DNA. In some embodiments, the therapeutic compositions are formulated to comprise a synthetic perilymph solution. An exemplary synthetic perilymph solution includes 20-200 mM NaCl; 1-5 mM KCl; 0.1-10 mM CaCl₂; 1-10 mM glucose; 2-50 mM HEPES, having a pH of between about 6 and about 9.

Also provided are kits including any of the compositions described herein. In some embodiments, a kit can include a solid composition (e.g., a lyophilized composition including the at least two different vectors described herein) and a liquid for solubilizing the lyophilized composition. In some embodiments, a kit can include a pre-loaded syringe including any of the compositions described herein.

In some embodiments, the kit includes a vial comprising any of the compositions described herein (e.g., formulated as an aqueous composition, e.g., an aqueous pharmaceutical composition).

In some embodiments, the kits can include instructions for performing any of the methods described herein.

### Devices and Surgical Methods

Provided herein are therapeutic delivery systems for treating hearing loss (e.g., acoustic neuromas/vestibular schwannomas and associated-hearing loss). In one aspect, the therapeutic delivery systems include i) a medical device capable of creating one or a plurality of incisions in a round window membrane of an inner ear of a human subject in need thereof, and ii) an effective dose of a composition (e.g., any of the compositions described herein). In some embodiments, the medical device includes a plurality of micro-needles.

Also provided herein are surgical methods for treatment of hearing loss (e.g., acoustic neuromas/vestibular schwannomas and associated-hearing loss). In some embodiments, the methods include the steps of: introducing into a cochlea of a human subject a first incision at a first incision point; and administering intra-cochlearly a therapeutically effective amount of any of the compositions provided herein. In some embodiments, the composition is administered to the subject at the first incision point. In some embodiments, the composition is administered to the subject into or through the first incision.

In some embodiments of any of the methods described herein, any of the compositions described herein is administered to the subject into or through the cochlea oval window membrane. In some embodiments of any of the methods described herein, any of the compositions described herein is administered to the subject into or through the cochlea round window membrane. In some embodiments of any of the methods described herein, the composition is administered using a medical device capable of creating a plurality of incisions in the round window membrane. In some embodiments, the medical device includes a plurality of micro-needles. In some embodiments, the medical device includes a plurality of micro-needles including a generally circular first aspect, where each micro-needle has a diameter of at least about 10 microns. In some embodiments, the medical device includes a base and/or a reservoir capable of holding the composition. In some embodiments, the medical device includes a plurality of hollow micro-needles individually including a lumen capable of transferring the composition. In some embodiments, the medical device includes a means for generating at least a partial vacuum.

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations that become evident as a result of the teaching provided herein.

Without further description, it is believed that one of ordinary skill in the art can, using the preceding description and the following illustrative examples, make and utilize the compositions of the present invention and practice the claimed methods. The following working examples specifically point out various aspects of the present invention, and are not to be construed as limiting in any way the remainder of the disclosure.

### EXAMPLES

### Example 1. Construction of Viral Vectors

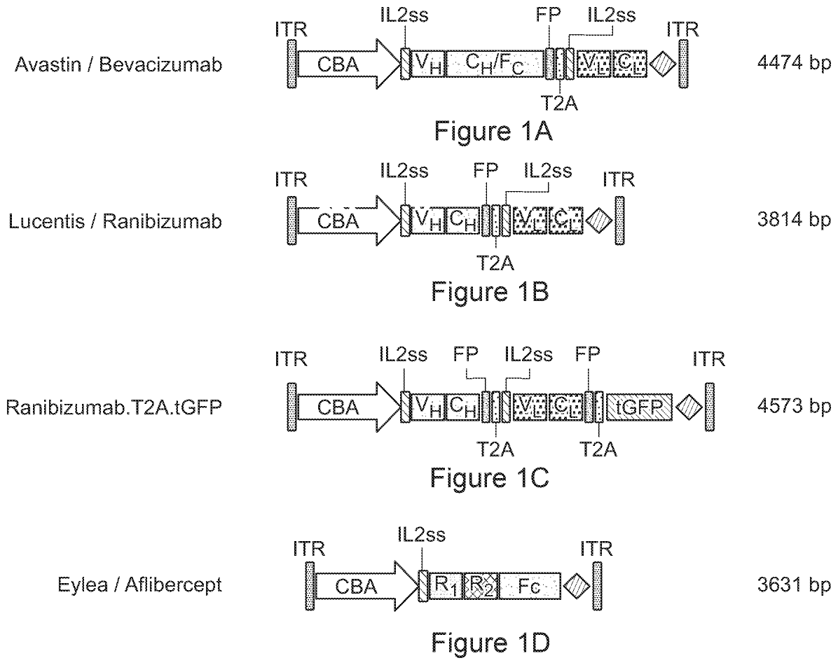

Four different recombinant AAV vectors were generated and are shown in FIGS. 1A-D.

The vector in FIG. 1A is an exemplary AAV vector of 4474 bp (SEQ ID NO: 35) that includes the following sub-sequences going in the 5′ to 3′ direction:

CCTGCAGGCAGCTGCGCGCTCGCTCGCTCACTGAGGCCGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTGCGGCCGCACGCGT(5′
ITR; SEQ ID NO: 36);
GACATTGATTATTGACTAGTTATTAATAGTAATCAATTACGGGGTCATTA
GTTCATAGCCCATATATGGAGTTCCGCGTTACATAACTTACGGTAAATGG
CCCGCCTGGCTGACCGCCCAACGACCCCCGCCCATTGACGTCAATAATGA
CGTATGTTCCCATAGTAACGCCAATAGGGACTTTCCATTGACGTCAATGG
GTGGACTATTTACGGTAAACTGCCCACTTGGCAGTACATCAAGTGTATCA
TATGCCAAGTACGCCCCCTATTGACGTCAATGACGGTAAATGGCCCGCCT
GGCATTATGCCCAGTACATGACCTTATGGGACTTTCCTACTTGGCAGTAC
ATCTACGTATTAGTCATCGCTATTACCATGGGTCGAGGTGAGCCCCACGT
TCTGCTTCACTCTCCCCATCTCCCCCCCCTCCCCACCCCCAATTTTGTAT
TTATTTATTTTTTAATTATTTTGTGCAGCGATGGGGGCGGGGGGGGGGGG
GGCGCGCGCCAGGCGGGGCGGGGCGGGGCGAGGGGCGGGGCGGGGCGAGG
CGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGC
GGCGGGCGGGAGTCGCTGCGTTGCCTTCGCCCCGTGCCCCGCTCCGCGCC
GCCTCGCGCCGCCCGCCCCGGCTCTGACTGACCGCGTTACTCCCACAGGT
GAGCGGGCGGGACGGCCCTTCTCCTCCGGGCTGTAATTAGCGCTTGGTTT
AATGACGGCTCGTTTCTTTTCTGTGGCTGCGTGAAAGCCTTAAAGGGCTC
CGGGAGGGCCCTTTGTGCGGGGGGGAGCGGCTCGGGGGGTGCGTGCGTGT
GTGTGTGCGTGGGGAGCGCCGCGTGCGGCCCGCGCTGCCCGGCGGCTGTG
AGCGCTGCGGGCGCGGCGCGGGGCTTTGTGCGCTCCGCGTGTGCGCGAGG
GGAGCGCGGCCGGGGGCGGTGCCCCGCGGTGCGGGGGGGCTGCGAGGGGA
ACAAAGGCTGCGTGCGGGGTGTGTGCGTGGGGGGGTGAGCAGGGGGTGTG
GGCGCGGCGGTCGGGCTGTAACCCCCCCCTGCACCCCCCTCCCCGAGTTG
CTGAGCACGGCCCGGCTTCGGGTGCGGGGCTCCGTGCGGGGCGTGGCGCG
GGGCTCGCCGTGCCGGGCGGGGGGTGGCGGCAGGTGGGGGTGCCGGGCGG
GGCGGGGCCGCCTCGGGCCGGGGAGGGCTCGGGGGAGGGGCGCGGCGGCC
CCCGGAGCGCCGGCGGCTGTCGAGGCGCGGCGAGCCGCAGCCATTGCCTT
TTATGGTAATCGTGCGAGAGGGCGCAGGGACTTCCTTTGTCCCAAATCTG
TGCGGAGCCGAAATCTGGGAGGCGCCGCCGCACCCCCTCTAGCGGGCGCG
GGGCGAAGCGGTGCGGCGCCGGCAGGAAGGAAATGGGCGGGGAGGGCCTT
CGTGCGTCGCCGCGCCGCCGTCCCCTTCTCCCTCTCCAGCCTCGGGGCTG
TCCGCGGGGGGACGGCTGCCTTCGGGGGGGACGGGGCAGGGCGGGGTTCG
GCTTCTGGCGTGTGACCGGCGGCTCTAGAGCCTCTGCTAACCATGTTCAT
GCCTTCTTCTTTTTCCTACAG(CBA sequence; SEQ ID NO:
37);
CTCCTGGGCAACGTGCTGGTTATTGTGACCGGTGCCACC(spacer;
SEQ ID NO: 38);
ATGTACCGGATGCAGCTGCTGAGCTGTATCGCCCTGTCTCTGGCCCTGGT
CACCAATTCT (IL-2 secretion signal sequence; SEQ ID
NO: 39);
GAGGTGCAGCTGGTGGAATCTGGCGGCGGACTTGTTCAACCTGGCGGCTC
TCTGAGACTGAGCTGTGCCGCTTCTGGCTACACCTTCACCAACTACGGCA
TGAACTGGGTCCGACAGGCCCCTGGCAAAGGCCTTGAATGGGTCGGATGG
ATCAACACCTACACCGGCGAGCCAACATACGCCGCCGACTTCAAGCGGAG
ATTCACCTTCAGCCTGGACACCAGCAAGAGCACCGCCTACCTGCAGATGA
ACAGCCTGAGAGCCGAGGACACCGCCGTGTACTACTGCGCCAAGTATCCC
CACTACTACGGCAGCAGCCACTGGTACTTTGACGTGTGGGGACAGGGCAC
ACTGGTCACAGTGTCTAGCGCCTCTACAAAGGGCCCCAGCGTTTTCCCAC
TGGCTCCTAGCAGCAAGTCTACCAGCGGAGGAACAGCCGCTCTGGGCTGT
CTGGTCAAGGACTACTTTCCCGAGCCTGTGACCGTGTCCTGGAATTCTGG
CGCTCTGACAAGCGGCGTGCACACCTTTCCAGCTGTGCTGCAAAGCAGCG
GCCTGTACTCTCTGAGCAGCGTCGTGACAGTGCCAAGCAGCTCTCTGGGC
ACCCAGACCTACATCTGCAATGTGAACCACAAGCCTAGCAACACCAAGGT
GGACAAGAAGGTGGAACCCAAGAGCTGCGACAAGACCCACACCTGTCCTC
CATGTCCTGCTCCAGAACTGCTCGGCGGACCTTCCGTGTTCCTGTTTCCT
CCAAAGCCTAAGGACACCCTGATGATCAGCAGAACCCCTGAAGTGACCTG
CGTGGTGGTGGATGTGTCCCACGAGGATCCCGAAGTGAAGTTCAATTGGT
ACGTGGACGGCGTGGAAGTGCACAACGCCAAGACCAAGCCTAGAGAGGAA
CAGTACAACAGCACCTACAGAGTGGTGTCCGTGCTGACCGTGCTGCACCA
GGATTGGCTGAACGGCAAAGAGTACAAGTGCAAGGTGTCCAACAAGGCCC
TGCCTGCTCCTATCGAGAAAACCATCAGCAAGGCCAAGGGCCAGCCTAGG
GAACCCCAGGTTTACACACTGCCTCCAAGCCGGGAAGAGATGACCAAGAA
CCAGGTGTCCCTGACCTGCCTCGTGAAGGGCTTCTACCCTTCCGATATCG
CCGTGGAATGGGAGAGCAATGGCCAGCCAGAGAACAACTACAAGACAACC
CCTCCTGTGCTGGACAGCGACGGCTCATTCTTCCTGTACAGCAAGCTGAC
AGTGGACAAGTCCAGATGGCAGCAGGGCAACGTGTTCAGCTGCAGCGTGA
TGCACGAGGCCCTGCACAACCACTACACCCAGAAGTCTCTGAGCCTGTCT
CCTGGCAAG(sequence encoding heavy chain of
bevacizumab; SEQ ID NO: 40);
CGGAAGAGAAGA(linker sequence; SEQ ID NO: 41);
GGCTCTGGCGAAGGCAGAGGCAGCCTGCTTACATGTGGCGACGTGGAAGA
GAACCCCGGACCT(T2A sequence; SEQ ID NO: 42);
ATGTATAGAATGCAGCTCCTGTCCTGCATTGCCCTGAGCCTGGCTCTCGT
GACCAACAGC(IL-2 secretion signal sequence; SEQ ID
NO: 43);
GACATCCAGATGACACAGAGCCCCAGCAGCCTGTCTGCCTCTGTGGGAGA
CAGAGTGACCATCACCTGTAGCGCCAGCCAGGACATCTCCAACTACCTGA
ACTGGTATCAGCAAAAGCCCGGCAAGGCCCCTAAGGTGCTGATCTACTTC
ACAAGCAGCCTGCACTCCGGCGTGCCCAGCAGATTTTCTGGCTCTGGCAG
CGGCACCGACTTCACCCTGACCATATCTAGCCTGCAGCCTGAGGACTTCG
CCACCTACTACTGCCAGCAGTACAGCACCGTGCCTTGGACATTTGGCCAG
GGCACAAAGGTGGAAATCAAGCGGACTGTGGCCGCTCCTAGCGTGTTCAT
CTTTCCACCTAGCGACGAGCAGCTGAAGTCTGGCACAGCCTCTGTCGTGT
GCCTGCTGAACAACTTCTACCCCAGAGAAGCCAAGGTGCAGTGGAAAGTG
GACAATGCCCTGCAGAGCGGCAACAGCCAAGAGAGCGTGACAGAGCAGGA
CTCCAAGGATAGCACCTATAGCCTGAGCAGCACCCTGACACTGAGCAAGG
CCGACTACGAGAAGCACAAAGTGTACGCCTGCGAAGTGACCCACCAGGGC
CTTTCTAGCCCTGTGACCAAGAGCTTCAACCGGGGCGAATGTTAA
(sequence encoding light chain of bevacizumab; SEQ
ID NO: 44);
GAGCTCGCTGATCAGCCTCGA(linker sequence; SEQ ID
NO: 45);
CTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGA
GGAAATTGCATCGCATTGTCTGAGTAGGTGTCATTCTATTCTGGGGGGTG
GGGTGGGGCAGGACAGCAAGGGGGAGGATTGGGAAGACAATAGCAGGCAT
GCTGGGGATGCGGTGGGCTCTATGG(bovine growth hormone
polyA tail sequence; SEQ ID NO: 46);
AAGCTTGAATTCAGCTGACGTGCCTCGGACCGCT(linker
sequence; SEQ ID NO: 47);
and
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGCTGCCTGCAGG(3′ ITR;
SEQ ID NO: 48).

The IL-2 signal sequence encoded by each of SEQ ID NOs: 39 and 43 is MYRMQLLSCIALSLALVTNS (SEQ ID NO: 49). The T2A sequence encoded by SEQ ID NO: 42 is GSGEGRGSLLTCGDVEENPGP (SEQ ID NO: 50). SEQ ID NO: 40 encodes the heavy chain of bevacizumab (SEQ ID NO: 6). SEQ ID NO: 44 encodes the light chain of bevacizumab (SEQ ID NO: 5). The last three nucleotides in SEQ ID NO: 44 are a stop codon.

The vector in FIG. 1B is an exemplary AAV vector of 3814 bp (SEQ ID NO: 51) that includes the following sub-sequences going in the 5′ to 3′ direction:

CCTGCAGGCAGCTGCGCGCTCGCTCGCTCACTGAGGCCGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTGCGGCCGCACGCGT(3′
ITR; SEQ ID NO: 36);
GACATTGATTATTGACTAGTTATTAATAGTAATCAATTACGGGGTCATTA
GTTCATAGCCCATATATGGAGTTCCGCGTTACATAACTTACGGTAAATGG
CCCGCCTGGCTGACCGCCCAACGACCCCCGCCCATTGACGTCAATAATGA
CGTATGTTCCCATAGTAACGCCAATAGGGACTTTCCATTGACGTCAATGG
GTGGACTATTTACGGTAAACTGCCCACTTGGCAGTACATCAAGTGTATCA
TATGCCAAGTACGCCCCCTATTGACGTCAATGACGGTAAATGGCCCGCCT
GGCATTATGCCCAGTACATGACCTTATGGGACTTTCCTACTTGGCAGTAC
ATCTACGTATTAGTCATCGCTATTACCATGGGTCGAGGTGAGCCCCACGT
TCTGCTTCACTCTCCCCATCTCCCCCCCCTCCCCACCCCCAATTTTGTAT
TTATTTATTTTTTAATTATTTTGTGCAGCGATGGGGGCGGGGGGGGGGGG
GGCGCGCGCCAGGCGGGGCGGGGCGGGGCGAGGGGCGGGGCGGGGCGAGG
CGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGC
GGCGGGCGGGAGTCGCTGCGTTGCCTTCGCCCCGTGCCCCGCTCCGCGCC
GCCTCGCGCCGCCCGCCCCGGCTCTGACTGACCGCGTTACTCCCACAGGT
GAGCGGGCGGGACGGCCCTTCTCCTCCGGGCTGTAATTAGCGCTTGGTTT
AATGACGGCTCGTTTCTTTTCTGTGGCTGCGTGAAAGCCTTAAAGGGCTC
CGGGAGGGCCCTTTGTGCGGGGGGGAGCGGCTCGGGGGGTGCGTGCGTGT
GTGTGTGCGTGGGGAGCGCCGCGTGCGGCCCGCGCTGCCCGGCGGCTGTG
AGCGCTGCGGGCGCGGCGCGGGGCTTTGTGCGCTCCGCGTGTGCGCGAGG
GGAGCGCGGCCGGGGGCGGTGCCCCGCGGTGCGGGGGGGCTGCGAGGGGA
ACAAAGGCTGCGTGCGGGGTGTGTGCGTGGGGGGGTGAGCAGGGGGTGTG
GGCGCGGCGGTCGGGCTGTAACCCCCCCCTGCACCCCCCTCCCCGAGTTG
CTGAGCACGGCCCGGCTTCGGGTGCGGGGCTCCGTGCGGGGCGTGGCGCG
GGGCTCGCCGTGCCGGGCGGGGGGTGGCGGCAGGTGGGGGTGCCGGGCGG
GGCGGGGCCGCCTCGGGCCGGGGAGGGCTCGGGGGAGGGGCGCGGCGGCC
CCCGGAGCGCCGGCGGCTGTCGAGGCGCGGCGAGCCGCAGCCATTGCCTT
TTATGGTAATCGTGCGAGAGGGCGCAGGGACTTCCTTTGTCCCAAATCTG
TGCGGAGCCGAAATCTGGGAGGCGCCGCCGCACCCCCTCTAGCGGGCGCG
GGGCGAAGCGGTGCGGCGCCGGCAGGAAGGAAATGGGCGGGGAGGGCCTT
CGTGCGTCGCCGCGCCGCCGTCCCCTTCTCCCTCTCCAGCCTCGGGGCTG
TCCGCGGGGGGACGGCTGCCTTCGGGGGGGACGGGGCAGGGCGGGGTTCG
GCTTCTGGCGTGTGACCGGCGGCTCTAGAGCCTCTGCTAACCATGTTCAT
GCCTTCTTCTTTTTCCTACAG(CBA sequence; SEQ ID NO: 37);
CTCCTGGGCAACGTGCTGGTTATTGTGACCGGTGCCACC(linker
sequence; SEQ ID NO: 38);
ATGTACCGGATGCAGCTGCTGAGCTGTATCGCCCTGTCTCTGGCCCTGGT
CACCAATTCT(IL-2 secretion signal sequence; SEQ ID
NO: 39);
GAGGTGCAGCTGGTGGAATCTGGCGGCGGACTTGTTCAACCTGGCGGCTC
TCTGAGACTGAGCTGTGCCGCTTCTGGCTACGACTTCACCCACTACGGCA
TGAACTGGGTCCGACAGGCCCCTGGCAAAGGCCTTGAATGGGTCGGATGG
ATCAACACCTACACCGGCGAGCCAACATACGCCGCCGACTTCAAGCGGAG
ATTCACCTTCAGCCTGGACACCAGCAAGAGCACCGCCTACCTGCAGATGA
ACAGCCTGAGAGCCGAGGACACCGCCGTGTACTACTGCGCCAAGTATCCC
TACTACTACGGCACCAGCCACTGGTACTTTGACGTGTGGGGACAGGGCAC
ACTGGTCACAGTGTCTAGCGCCTCTACAAAGGGCCCCAGCGTTTTCCCAC
TGGCTCCTAGCAGCAAGTCTACCAGCGGAGGAACAGCCGCTCTGGGCTGT
CTGGTCAAGGACTACTTTCCCGAGCCTGTGACCGTGTCCTGGAATTCTGG
CGCTCTGACAAGCGGCGTGCACACCTTTCCAGCTGTGCTGCAAAGCAGCG
GCCTGTACTCTCTGAGCAGCGTCGTGACAGTGCCAAGCAGCTCTCTGGGC
ACCCAGACCTACATCTGCAATGTGAACCACAAGCCTAGCAACACCAAGGT
GGACAAGAAGGTGGAACCCAAGAGCTGCGACAAGACCCACACCGGCAAG
(sequence encoding ranibizumab heavy chain; SEQ ID
NO: 52);
CGGAAGAGAAGA(linker sequence; SEQ ID NO: 41);
GGCTCTGGCGAAGGCAGAGGCAGCCTGCTTACATGTGGCGACGTGGAAGA
GAACCCCGGACCT(T2A sequence; SEQ ID NO: 42);
ATGTATAGAATGCAGCTCCTGTCCTGCATTGCCCTGAGCCTGGCTCTCGT
GACCAACAGC(IL-2 signal secretion sequence; SEQ ID
NO: 43);
GACATCCAGCTGACACAGAGCCCCAGCAGCCTGTCTGCCTCTGTGGGAGA
CAGAGTGACCATCACCTGTAGCGCCAGCCAGGACATCTCCAACTACCTGA
ACTGGTATCAGCAAAAGCCCGGCAAGGCCCCTAAGGTGCTGATCTACTTC
ACAAGCAGCCTGCACTCCGGCGTGCCCAGCAGATTTTCTGGCTCTGGCAG
CGGCACCGACTTCACCCTGACCATATCTAGCCTGCAGCCTGAGGACTTCG
CCACCTACTACTGCCAGCAGTACAGCACCGTGCCTTGGACATTTGGCCAG
GGCACAAAGGTGGAAATCAAGCGGACTGTGGCCGCTCCTAGCGTGTTCAT
CTTTCCACCTAGCGACGAGCAGCTGAAGTCTGGCACAGCCTCTGTCGTGT
GCCTGCTGAACAACTTCTACCCCAGAGAAGCCAAGGTGCAGTGGAAAGTG
GACAATGCCCTGCAGAGCGGCAACAGCCAAGAGAGCGTGACAGAGCAGGA
CTCCAAGGATAGCACCTATAGCCTGAGCAGCACCCTGACACTGAGCAAGG
CCGACTACGAGAAGCACAAAGTGTACGCCTGCGAAGTGACCCACCAGGGC
CTTTCTAGCCCTGTGACCAAGAGCTTCAACCGGGGCGAATGTTAA
(sequence encoding ranibizumab light chain; SEQ ID
NO: 53).
GAGCTCGCTGATCAGCCTCGA(linker sequence; SEQ ID NO:
45);
CTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGA
GGAAATTGCATCGCATTGTCTGAGTAGGTGTCATTCTATTCTGGGGGGTG
GGGTGGGGCAGGACAGCAAGGGGGAGGATTGGGAAGACAATAGCAGGCAT
GCTGGGGATGCGGTGGGCTCTATGG(bovine growth hormone
polyA tail sequence; SEQ ID NO: 46);
and
AAGCTTGAATTCAGCTGACGTGCCTCGGACCGCT(linker; SEQ ID
NO: 47);
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGCTGCCTGCAGG(SEQ ID
NO: 48).

The IL-2 signal sequence encoded by each of SEQ ID NOs: 39 and 43 is MYRMQLLSCIALSLALVTNS (SEQ ID NO: 49). The T2A sequence encoded by SEQ ID NO: 42 is GSGEGRGSLLTCGDVEENPGP (SEQ ID NO: 50). SEQ ID NO: 52 encodes the heavy chain of ranibizumab

SEQ ID NO: 53
(EVQLVESGGGLVQPGGSLRLSCAASGYDFTHYGMNWVRQAPGKGLEWVG
WINTYTGEPTYAADFKRRFTFSLDTSKSTAYLQMNSLRAEDTAVYYCAKY
PYYYGTSHWYFDVWGQGTLVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKKVEPKSCDKTHTGK; SEQ ID NO: 54).

encodes the light chain of bevacizumab (SEQ ID NO: 7). The last three nucleotides in SEQ ID NO: 53 are a stop codon.

FIG. 1C is an exemplary AAV vector of 4573 bp (SEQ ID NO: 55) that includes the following sub-sequences going in the 5′ to 3′ direction:

CCTGCAGGCAGCTGCGCGCTCGCTCGCTCACTGAGGCCGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTGCGGCCGCACGCGT(5′
ITR; SEQ ID NO: 36);
GACATTGATTATTGACTAGTTATTAATAGTAATCAATTACGGGGTCATTA
GTTCATAGCCCATATATGGAGTTCCGCGTTACATAACTTACGGTAAATGG
CCCGCCTGGCTGACCGCCCAACGACCCCCGCCCATTGACGTCAATAATGA
CGTATGTTCCCATAGTAACGCCAATAGGGACTTTCCATTGACGTCAATGG
GTGGACTATTTACGGTAAACTGCCCACTTGGCAGTACATCAAGTGTATCA
TATGCCAAGTACGCCCCCTATTGACGTCAATGACGGTAAATGGCCCGCCT
GGCATTATGCCCAGTACATGACCTTATGGGACTTTCCTACTTGGCAGTAC
ATCTACGTATTAGTCATCGCTATTACCATGGGTCGAGGTGAGCCCCACGT
TCTGCTTCACTCTCCCCATCTCCCCCCCCTCCCCACCCCCAATTTTGTAT
TTATTTATTTTTTAATTATTTTGTGCAGCGATGGGGGCGGGGGGGGGGGG
GGCGCGCGCCAGGCGGGGCGGGGCGGGGCGAGGGGCGGGGCGGGGCGAGG
CGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGC
GGCGGGCGGGAGTCGCTGCGTTGCCTTCGCCCCGTGCCCCGCTCCGCGCC
GCCTCGCGCCGCCCGCCCCGGCTCTGACTGACCGCGTTACTCCCACAGGT
GAGCGGGCGGGACGGCCCTTCTCCTCCGGGCTGTAATTAGCGCTTGGTTT
AATGACGGCTCGTTTCTTTTCTGTGGCTGCGTGAAAGCCTTAAAGGGCTC
CGGGAGGGCCCTTTGTGCGGGGGGGAGCGGCTCGGGGGGTGCGTGCGTGT
GTGTGTGCGTGGGGAGCGCCGCGTGCGGCCCGCGCTGCCCGGCGGCTGTG
AGCGCTGCGGGCGCGGCGCGGGGCTTTGTGCGCTCCGCGTGTGCGCGAGG
GGAGCGCGGCCGGGGGCGGTGCCCCGCGGTGCGGGGGGGCTGCGAGGGGA
ACAAAGGCTGCGTGCGGGGTGTGTGCGTGGGGGGGTGAGCAGGGGGTGTG
GGCGCGGCGGTCGGGCTGTAACCCCCCCCTGCACCCCCCTCCCCGAGTTG
CTGAGCACGGCCCGGCTTCGGGTGCGGGGCTCCGTGCGGGGCGTGGCGCG
GGGCTCGCCGTGCCGGGCGGGGGGTGGCGGCAGGTGGGGGTGCCGGGCGG
GGCGGGGCCGCCTCGGGCCGGGGAGGGCTCGGGGGAGGGGCGCGGCGGCC
CCCGGAGCGCCGGCGGCTGTCGAGGCGCGGCGAGCCGCAGCCATTGCCTT
TTATGGTAATCGTGCGAGAGGGCGCAGGGACTTCCTTTGTCCCAAATCTG
TGCGGAGCCGAAATCTGGGAGGCGCCGCCGCACCCCCTCTAGCGGGCGCG
GGGCGAAGCGGTGCGGCGCCGGCAGGAAGGAAATGGGCGGGGAGGGCCTT
CGTGCGTCGCCGCGCCGCCGTCCCCTTCTCCCTCTCCAGCCTCGGGGCTG
TCCGCGGGGGGACGGCTGCCTTCGGGGGGGACGGGGCAGGGCGGGGTTCG
GCTTCTGGCGTGTGACCGGCGGCTCTAGAGCCTCTGCTAACCATGTTCAT
GCCTTCTTCTTTTTCCTACAG(CBA sequence; SEQ ID NO: 37);
CTCCTGGGCAACGTGCTGGTTATTGTGACCGGTGCCACC(linker
sequence; SEQ ID NO: 38);
ATGTACCGGATGCAGCTGCTGAGCTGTATCGCCCTGTCTCTGGCCCTGGT
CACCAATTCT(IL-2 secretion signal sequence; SEQ ID
NO: 39);
GAGGTGCAGCTGGTGGAATCTGGCGGCGGACTTGTTCAACCTGGCGGCTC
TCTGAGACTGAGCTGTGCCGCTTCTGGCTACGACTTCACCCACTACGGCA
TGAACTGGGTCCGACAGGCCCCTGGCAAAGGCCTTGAATGGGTCGGATGG
ATCAACACCTACACCGGCGAGCCAACATACGCCGCCGACTTCAAGCGGAG
ATTCACCTTCAGCCTGGACACCAGCAAGAGCACCGCCTACCTGCAGATGA
ACAGCCTGAGAGCCGAGGACACCGCCGTGTACTACTGCGCCAAGTATCCC
TACTACTACGGCACCAGCCACTGGTACTTTGACGTGTGGGGACAGGGCAC
ACTGGTCACAGTGTCTAGCGCCTCTACAAAGGGCCCCAGCGTTTTCCCAC
TGGCTCCTAGCAGCAAGTCTACCAGCGGAGGAACAGCCGCTCTGGGCTGT
CTGGTCAAGGACTACTTTCCCGAGCCTGTGACCGTGTCCTGGAATTCTGG
CGCTCTGACAAGCGGCGTGCACACCTTTCCAGCTGTGCTGCAAAGCAGCG
GCCTGTACTCTCTGAGCAGCGTCGTGACAGTGCCAAGCAGCTCTCTGGGC
ACCCAGACCTACATCTGCAATGTGAACCACAAGCCTAGCAACACCAAGGT
GGACAAGAAGGTGGAACCCAAGAGCTGCGACAAGACCCACACCGGCAAG
(sequence encoding ranibizumab heavy chain; SEQ ID
NO: 52);
CGGAAGAGAAGA(linker sequence; SEQ ID NO: 41);
GGCTCTGGCGAAGGCAGAGGCAGCCTGCTTACATGTGGCGACGTGGAAGA
GAACCCCGGACCT(T2A sequence;SEQ ID NO: 42);
ATGTATAGAATGCAGCTCCTGTCCTGCATTGCCCTGAGCCTGGCTCTCGT
GACCAACAGC(IL-2 signal secretion sequence; SEQ ID
NO: 43);
GACATCCAGCTGACACAGAGCCCCAGCAGCCTGTCTGCCTCTGTGGGAGA
CAGAGTGACCATCACCTGTAGCGCCAGCCAGGACATCTCCAACTACCTGA
ACTGGTATCAGCAAAAGCCCGGCAAGGCCCCTAAGGTGCTGATCTACTTC
ACAAGCAGCCTGCACTCCGGCGTGCCCAGCAGATTTTCTGGCTCTGGCAG
CGGCACCGACTTCACCCTGACCATATCTAGCCTGCAGCCTGAGGACTTCG
CCACCTACTACTGCCAGCAGTACAGCACCGTGCCTTGGACATTTGGCCAG
GGCACAAAGGTGGAAATCAAGCGGACTGTGGCCGCTCCTAGCGTGTTCAT
CTTTCCACCTAGCGACGAGCAGCTGAAGTCTGGCACAGCCTCTGTCGTGT
GCCTGCTGAACAACTTCTACCCCAGAGAAGCCAAGGTGCAGTGGAAAGTG
GACAATGCCCTGCAGAGCGGCAACAGCCAAGAGAGCGTGACAGAGCAGGA
CTCCAAGGATAGCACCTATAGCCTGAGCAGCACCCTGACACTGAGCAAGG
CCGACTACGAGAAGCACAAAGTGTACGCCTGCGAAGTGACCCACCAGGGC
CTTTCTAGCCCTGTGACCAAGAGCTTCAACCGGGGCGAATGT
(sequence encoding ranibizumab light chain; SEQ ID
NO: 56);
GGCTCCGGAGAGGGCAGAGGAAGTCTGCTAACATGCGGTGACGTCGAGGA
GAATCCTGGCCCA(linker sequence; SEQ ID NO: 57);
ATGGAGAGCGACGAGAGCGGCCTGCCCGCCATGGAGATCGAGTGCCGCAT
CACCGGCACCCTGAACGGCGTGGAGTTCGAGCTGGTGGGCGGCGGAGAGG
GCACCCCCGAGCAGGGCCGCATGACCAACAAGATGAAGAGCACCAAAGGC
GCCCTGACCTTCAGCCCCTACCTGCTGAGCCACGTGATGGGCTACGGCTT
CTACCACTTCGGCACCTACCCCAGCGGCTACGAGAACCCCTTCCTGCACG
CCATCAACAACGGCGGCTACACCAACACCCGCATCGAGAAGTACGAGGAC
GGCGGCGTGCTGCACGTGAGCTTCAGCTACCGCTACGAGGCCGGCCGCGT
GATCGGCGACTTCAAGGTGATGGGCACCGGCTTCCCCGAGGACAGCGTGA
TCTTCACCGACAAGATCATCCGCAGCAACGCCACCGTGGAGCACCTGCAC
CCCATGGGCGATAACGATCTGGATGGCAGCTTCACCCGCACCTTCAGCCT
GCGCGACGGCGGCTACTACAGCTCCGTGGTGGACAGCCACATGCACTTCA
AGAGCGCCATCCACCCCAGCATCCTGCAGAACGGGGGCCCCATGTTCGCC
TTCCGCCGCGTGGAGGAGGATCACAGCAACACCGAGCTGGGCATCGTGGA
GTACCAGCACGCCTTCAAGACCCCGGATGCAGATGCCGGTGAAGAATAA
(sequence encoding Turbo GFP; SEQ ID NO: 58);
GAGCTCGCTGATCAGCCTCGA(linker sequence; SEQ ID NO:
45);
CTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGA
GGAAATTGCATCGCATTGTCTGAGTAGGTGTCATTCTATTCTGGGGGGTG
GGGTGGGGCAGGACAGCAAGGGGGAGGATTGGGAAGACAATAGCAGGCAT
GCTGGGGATGCGGTGGGCTCTATGG(bovine growth hormone
polyA tail sequence; SEQ ID NO: 46);
AAGCTTGAATTCAGCTGACGTGCCTCGGACCGCT(linker
sequence; SEQ ID NO: 47);
and
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGCTGCCTGCAGG(3′ ITR;
SEQ ID NO: 48).

The IL-2 signal sequence encoded by each of SEQ ID NOs: 39 and 43 is MYRMQLLSCIALSLALVTNS (SEQ ID NO: 49). The T2A sequence encoded by SEQ ID NO: 42 is GSGEGRGSLLTCGDVEENPGP (SEQ ID NO: 50). SEQ ID NO: 52 encodes the heavy chain of ranibizumab (SEQ ID NO: 54). SEQ ID NO: 56 encodes the light chain of bevacizumab (SEQ ID NO: 7). SEQ ID NO: 58 encodes TurboGFP

(MESDESGLPAMEIECRITGTLNGVEFELVGGGEGTPEQGRMTNKMKSTKGA LTFSPYLLSHVMGYGFYHFGTYPSGYENPFLHAINNGGYTNTRIEKYEDGGV LHVSFSYRYEAGRVIGDFKVMGTGFPEDSVIFTDKIIRSNATVEHLHPMGDND LDGSFTRTFSLRDGGYYSSVVDSHMHFKSAIHPSILQNGGPMFAFRRVEEDHS NTELGIVEYQHAFKTPDADAGEE; SEQ ID NO: 59). The last three nucleotides in SEQ ID NO: 58 is a stop codon.

FIG. 1D is an exemplary AAV vector of 3631 bp (SEQ ID NO: 60) that includes the following sub-sequences going in the 5′ to 3′ direction:

CCTGCAGGCAGCTGCGCGCTCGCTCGCTCACTGAGGCCGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTGCGGCCGCACGCGT(5′
ITR; SEQ ID NO: 36);
GACATTGATTATTGACTAGTTATTAATAGTAATCAATTACGGGGTCATTA
GTTCATAGCCCATATATGGAGTTCCGCGTTACATAACTTACGGTAAATGG
CCCGCCTGGCTGACCGCCCAACGACCCCCGCCCATTGACGTCAATAATGA
CGTATGTTCCCATAGTAACGCCAATAGGGACTTTCCATTGACGTCAATGG
GTGGACTATTTACGGTAAACTGCCCACTTGGCAGTACATCAAGTGTATCA
TATGCCAAGTACGCCCCCTATTGACGTCAATGACGGTAAATGGCCCGCCT
GGCATTATGCCCAGTACATGACCTTATGGGACTTTCCTACTTGGCAGTAC
ATCTACGTATTAGTCATCGCTATTACCATGGGTCGAGGTGAGCCCCACGT
TCTGCTTCACTCTCCCCATCTCCCCCCCCTCCCCACCCCCAATTTTGTAT
TTATTTATTTTTTAATTATTTTGTGCAGCGATGGGGGCGGGGGGGGGGGG
GGCGCGCGCCAGGCGGGGCGGGGCGGGGCGAGGGGCGGGGCGGGGCGAGG
CGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGC
GGCGGGCGGGAGTCGCTGCGTTGCCTTCGCCCCGTGCCCCGCTCCGCGCC
GCCTCGCGCCGCCCGCCCCGGCTCTGACTGACCGCGTTACTCCCACAGGT
GAGCGGGCGGGACGGCCCTTCTCCTCCGGGCTGTAATTAGCGCTTGGTTT
AATGACGGCTCGTTTCTTTTCTGTGGCTGCGTGAAAGCCTTAAAGGGCTC
CGGGAGGGCCCTTTGTGCGGGGGGGAGCGGCTCGGGGGGTGCGTGCGTGT
GTGTGTGCGTGGGGAGCGCCGCGTGCGGCCCGCGCTGCCCGGCGGCTGTG
AGCGCTGCGGGCGCGGCGCGGGGCTTTGTGCGCTCCGCGTGTGCGCGAGG
GGAGCGCGGCCGGGGGCGGTGCCCCGCGGTGCGGGGGGGCTGCGAGGGGA
ACAAAGGCTGCGTGCGGGGTGTGTGCGTGGGGGGGTGAGCAGGGGGTGTG
GGCGCGGCGGTCGGGCTGTAACCCCCCCCTGCACCCCCCTCCCCGAGTTG
CTGAGCACGGCCCGGCTTCGGGTGCGGGGCTCCGTGCGGGGCGTGGCGCG
GGGCTCGCCGTGCCGGGCGGGGGGTGGCGGCAGGTGGGGGTGCCGGGCGG
GGCGGGGCCGCCTCGGGCCGGGGAGGGCTCGGGGGAGGGGCGCGGCGGCC
CCCGGAGCGCCGGCGGCTGTCGAGGCGCGGCGAGCCGCAGCCATTGCCTT
TTATGGTAATCGTGCGAGAGGGCGCAGGGACTTCCTTTGTCCCAAATCTG
TGCGGAGCCGAAATCTGGGAGGCGCCGCCGCACCCCCTCTAGCGGGCGCG
GGGCGAAGCGGTGCGGCGCCGGCAGGAAGGAAATGGGCGGGGAGGGCCTT
CGTGCGTCGCCGCGCCGCCGTCCCCTTCTCCCTCTCCAGCCTCGGGGCTG
TCCGCGGGGGGACGGCTGCCTTCGGGGGGGACGGGGCAGGGCGGGGTTCG
GCTTCTGGCGTGTGACCGGCGGCTCTAGAGCCTCTGCTAACCATGTTCAT
GCCTTCTTCTTTTTCCTACAG(CBA sequence; SEQ ID NO: 37);
CTCCTGGGCAACGTGCTGGTTATTGTGACCGGTGCCACC(spacer;
SEQ ID NO: 38);
ATGTACCGGATGCAGCTGCTGAGCTGTATCGCCCTGTCTCTGGCCCTGGT
CACCAATTCT(IL-2 secretion signal sequence; SEQ ID
NO: 39);
AGCGATACCGGCAGACCCTTCGTGGAAATGTACAGCGAGATCCCCGAGAT
CATCCACATGACCGAGGGCAGAGAGCTGGTCATCCCCTGCAGAGTGACAA
GCCCCAACATCACCGTGACTCTGAAGAAGTTCCCTCTGGACACACTGATC
CCCGACGGCAAGAGAATCATCTGGGACAGCCGGAAGGGCTTCATCATCAG
CAACGCCACCTACAAAGAGATCGGCCTGCTGACCTGTGAAGCCACCGTGA
ATGGCCACCTGTACAAGACCAACTACCTGACACACAGACAGACCAACACC
ATCATCGACGTGGTGCTGAGCCCTAGCCACGGCATTGAACTGTCTGTGGG
CGAGAAGCTGGTGCTGAACTGTACCGCCAGAACCGAGCTGAACGTGGGCA
TCGACTTCAACTGGGAGTACCCCAGCAGCAAGCACCAGCACAAGAAACTG
GTCAACCGGGACCTGAAAACCCAGAGCGGCAGCGAGATGAAGAAATTCCT
GAGCACCCTGACCATCGACGGCGTGACCAGATCTGACCAGGGCCTGTACA
CATGTGCCGCCAGCTCTGGCCTGATGACCAAGAAAAACAGCACCTTCGTG
CGGGTGCACGAGAAGGACAAGACCCACACCTGTCCTCCATGTCCTGCTCC
AGAACTGCTCGGCGGACCTTCCGTGTTCCTGTTTCCTCCAAAGCCTAAGG
ACACCCTGATGATCAGCAGAACCCCTGAAGTGACCTGCGTGGTGGTGGAT
GTGTCCCACGAGGATCCCGAAGTGAAGTTCAATTGGTACGTGGACGGCGT
GGAAGTGCACAACGCCAAGACCAAGCCTAGAGAGGAACAGTACAATAGCA
CCTACAGAGTGGTGTCCGTGCTGACCGTGCTGCACCAGGATTGGCTGAAC
GGCAAAGAGTACAAGTGCAAGGTGTCCAACAAGGCCCTGCCTGCTCCTAT
CGAGAAAACCATCTCCAAGGCCAAGGGCCAGCCTAGGGAACCCCAGGTTT
ACACACTGCCTCCAAGCAGGGACGAGCTGACAAAGAACCAGGTGTCCCTG
ACCTGCCTGGTCAAGGGCTTCTACCCTTCCGATATCGCCGTGGAATGGGA
GAGCAATGGCCAGCCTGAGAACAACTACAAGACAACCCCTCCTGTGCTGG
ACAGCGACGGCTCATTCTTCCTGTACAGCAAGCTGACAGTGGACAAGAGC
AGATGGCAGCAGGGCAACGTGTTCAGCTGCAGCGTGATGCACGAGGCCCT
GCACAACCACTACACCCAGAAGTCCCTGAGCCTGTCTCCTGGATAA
(sequence encoding a flibercept; SEQ ID NO: 61);
GAGCTCGCTGATCAGCCTCGA(linker sequence; SEQ ID NO:
45);
CTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGA
GGAAATTGCATCGCATTGTCTGAGTAGGTGTCATTCTATTCTGGGGGGTG
GGGTGGGGCAGGACAGCAAGGGGGAGGATTGGGAAGACAATAGCAGGCAT
GCTGGGGATGCGGTGGGCTCTATGG(bovine growth hormone
polyA tail sequence; SEQ ID NO: 46);
AAGCTTGAATTCAGCTGACGTGCCTCGGACCGCT(linker
sequence; SEQ ID NO: 47);
and
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGCTGCCTGCAGG(3′ ITR;
SEQ ID NO: 48).

The IL-2 signal sequence encoded by SEQ ID NO: 39 is MYRMQLLSCIALSLALVTNS (SEQ ID NO: 49). SEQ ID NO: 61 encodes aflibercept (SEQ ID NO: 12). The last three nucleotides in SEQ ID NO: 61 is a stop codon.

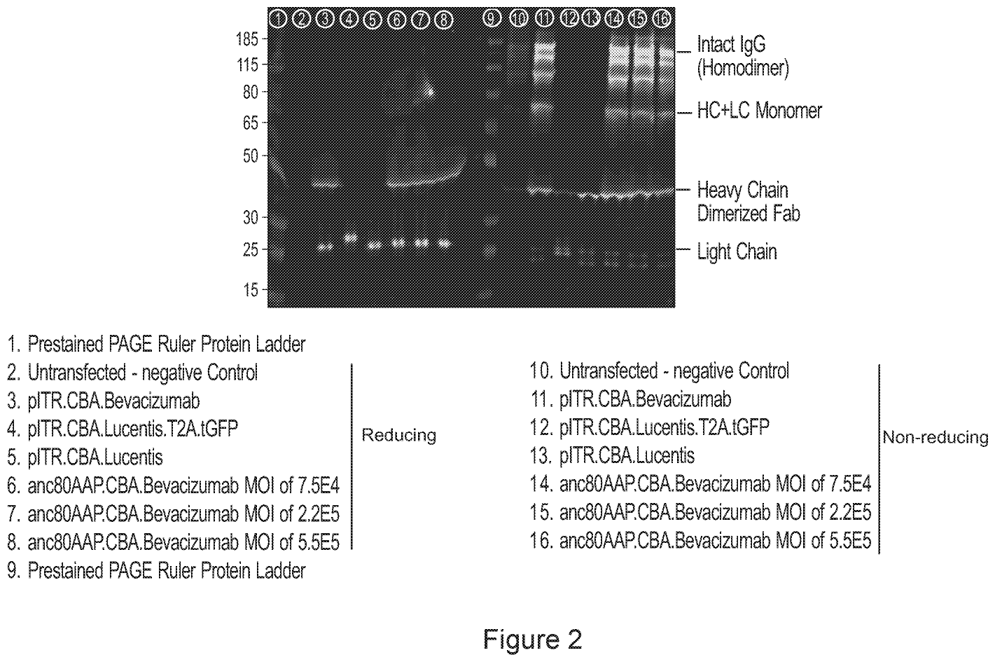

To determine protein expression driven by the AAV vectors shown in FIGS. 1A-1C, HEK293FT cells were seeded overnight at 7×10⁴ cells/well (400 μL per well) in wells of a 24-well plate. HEK293FT cells were transfected at ˜800 ng with the AAV vectors shown in FIGS. 1A-1D using a Jetprime Polypus reagent (used to generate the data in Lanes 2-5 and 10-13 of FIG. 2). HEK293FT cells were also seeded for six hours at 4×10⁴ cells/well (50 μL per well) in wells of a 96-well plate in the presence of 2 μM etoposide (used to generate the data in Lanes 6-8 and 14-16 of FIG. 2). The AAV vector shown in FIG. 1A was added into the media with a multiplicity of infection (MOI) of 7.5×10⁴, 2.2×10⁵, or 5.5×10⁵. The supernatant was harvested at 72 hours post-treatment from well and was loaded onto a 4-12% Bolt protein gel in reducing (lanes 2-8 of FIG. 2) and non-reducing conditions (lanes 10-16 of FIG. 2). An anti-ranibizumab antibody detecting the Fab region was used as a primary antibody, and anti-human IgG was used as the second antibody.

As shown in FIG. 2, the heavy chain and light chain ranibizumab were detected in Lanes 3 and 6-8, and intact ranibizumab (heterodimer) was detected in lanes 11 and 14-16.

### Example 2. Binding Activity of Anti-Human VEGF Monoclonal Antibodies

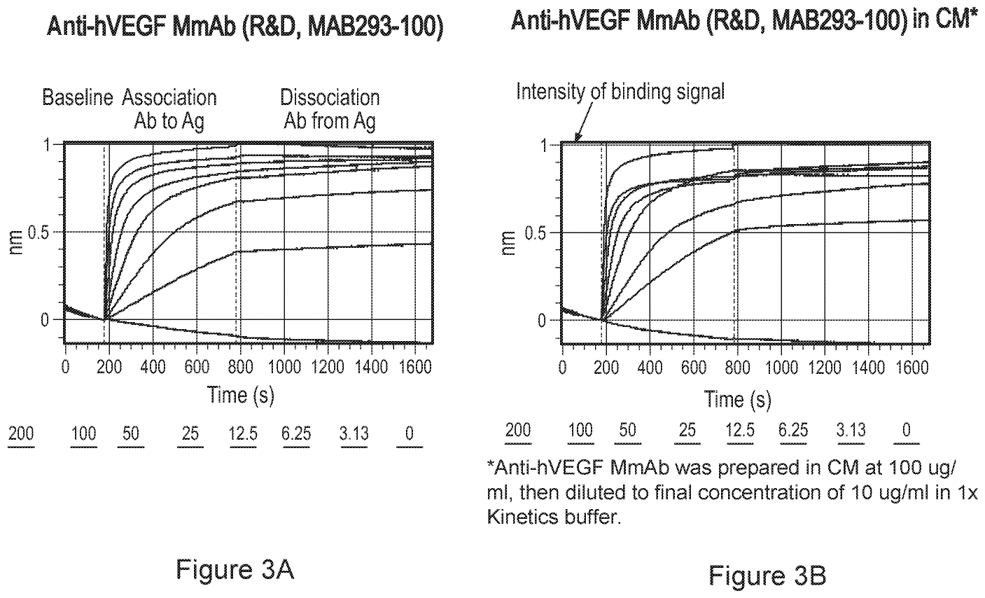

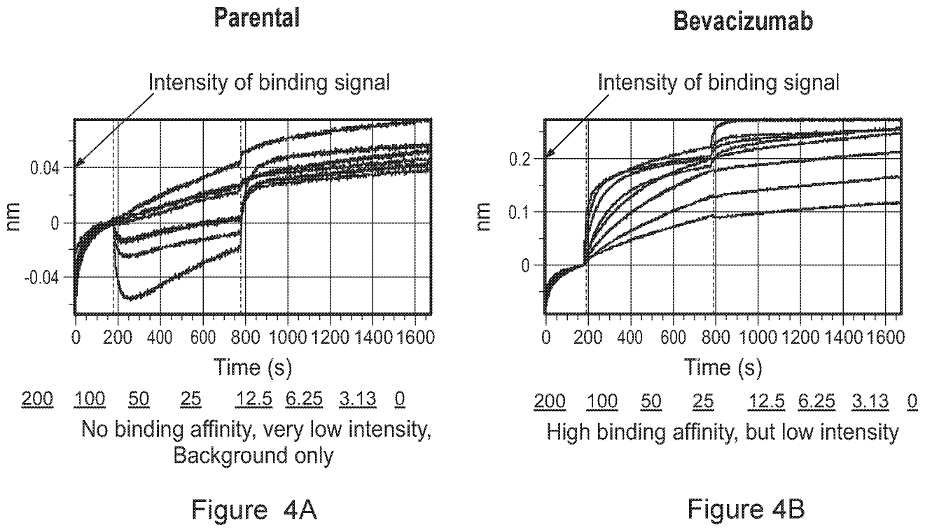

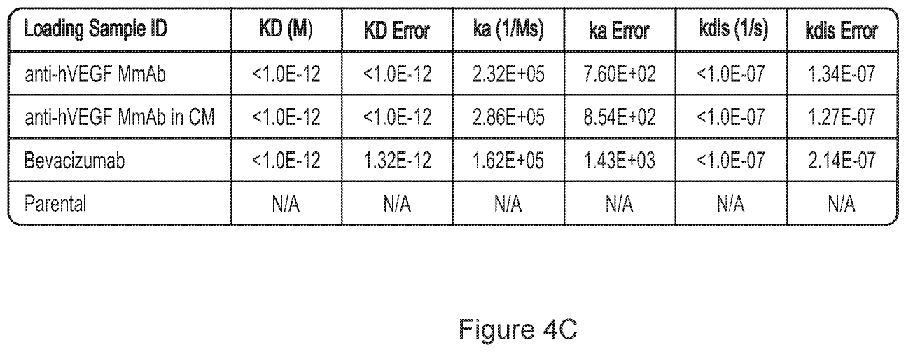

A set of experiments were performed to determine the binding activity of bevacizumab produced in HEK293FT cells following transfection with the AAV vector shown in FIG. 1A. A first set of control experiments were performed to calibrate the plasmon surface resonance instrumentation (using a mouse anti-human VEGF monoclonal antibody (anti-hVEGF MmAb; R&D, MAB293-100) in buffer or in conditioned medium (FIG. 3A and 3B, respectivey) using recombinant human VEGF as the binding agent. A second set of experiments were performed to determine the human VEGF-binding activity of control conditioned medium and conditioned medium from HEK293TF cells following transfection with the AAV vector shown in FIG. 1A (FIGS. 4A and 4B, respectively).

The samples, bevacizumab in medium from HEK293TF cells transfected with the AAV vector shown in FIG. 1A or conditioned medium), were prepared by diluting 1:10 in 1×kinetics buffer (Fortebio, 18-1105) into a 384-well sample plate. Anti-hVEGF MmAb (R&D, MAB293-100) was diluted at a concentration of 10 μg/mL as a positive control. The capture agent, recombinant human VEGF (R&D, 293-VE-010) was diluted in a series of 1:2 dilution ratio from 200 nM to 3.125 nM.

The binding affinities of the conditioned medium samples and mouse anti-human VEGF antibody (R&D) samples were measured in 1×kinetics buffer in Octet® HTX biosensor instrument. The binding features and K_{D} values were generated by the Octet® analysis software, Data Analysis HT10.0. As shown in FIGS. 3A-B, the K_{D} of anti-hVEGF MmAb in buffer was <1.0×10⁻¹² M, and the anti-hVEGF MmAb in conditioned medium was <1.0×1 0⁻¹² M. The conditioned medium itself had no binding affinity and very low intensity (background signal only) (FIG. 4A). In contrast, the conditioned medium including bevacizumab produced by HEK293TF cells transfected with the AAV vector shown in FIG. 1A had high binding affinity, but low intensity (FIG. 4B; K_{D} <1.0×10⁻¹² M). FIG. 4C shows a table of the loading samples and the respective K_{D}, K_{D} errors, equilibrium association constant (kₐ), and the dissociation (k_{dis}), and k_{dis} error.

In summary, the anti-hVEGF mouse antibody (R&D) showed high binding affinity (K_{D} was lower than measurable range of 1.0×10⁻¹² M). The bevacizumab conditioned medium sample showed high binding affinity (K_{D} was lower than measurable range). No K_{D} value could be extrapolated from the binding data of control conditioned medium sample.

In sum, these data show that the AAV vectors provided herein can result in expression and secretion of anti-VEGF antibodies and can be used to express anti-VEGF antibodies in the inner ear of a mammal.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. Section headings and any descriptions of materials, methods, and examples are illustrative only and not intended to be limiting.

1-112. (canceled)

113. An adeno-associated virus (AAV) vector comprising a nucleotide sequence that comprises:
(i) a first coding sequence that encodes a first polypeptide, wherein the first polypeptide comprises an antibody heavy chain variable domain operably linked to a first signal peptide, and
(ii) a second coding sequence that encodes a second polypeptide, wherein the second polypeptide comprises an antibody light chain variable domain operably linked to a second signal peptide,
wherein the first coding sequence comprises a sequence having at least 96% identity to SEQ ID NO: 40, and the second coding sequence comprises a sequence having at least 96% identity to SEQ ID NO: 44, or both, and
wherein the first and second polypeptides specifically bind to one or more mammalian VEGF proteins.

114. An adeno-associated virus (AAV) vector comprising a nucleotide sequence that comprises:
a coding sequence that encodes a polypeptide, wherein the polypeptide comprises one or more soluble vascular endothelial growth factor (VEGF) receptors operably linked to a signal peptide, wherein the coding sequence comprises a sequence having at least 96% identity to SEQ ID NO: 61, and wherein the polypeptide specifically binds to one or more mammalian VEGF proteins.

115. The AAV vector of claim 113, wherein the nucleotide sequence comprises a promoter, a Kozak sequence, or both.

116. The AAV vector of claim 115, wherein the promoter is an inducible promoter, a constitutive promoter, or a tissue-specific promoter.

117. The AAV vector of claim 116, wherein the promoter is a CAG promoter, a CBA promoter, or a CMV promoter.

118. The AAV vector of claim 113, wherein the nucleotide sequence further comprises a polyadenylation signal sequence.

119. The AAV vector of claim 113, wherein the first and second polypeptides comprise:
(i) the amino sequence of SEQ ID NO: 6,
(ii) the amino sequence of SEQ ID NO: 5, or
(iii) both (i) and (ii).

120. The AAV vector of claim 119, wherein the first and second polypeptides comprise bevacizumab.

121. The AAV vector of claim 113, wherein the nucleotide sequence comprises one or more sequences encoding a thosea asigna virus 2A (T2A) peptide.

122. The AAV vector of claim 114, wherein the soluble VEGF receptor comprises:
(i) a portion of an extracellular region of VEGF receptor-1 (VEGFR-1);
(ii) a portion of an extracellular region of VEGF receptor-2 (VEGFR-2);
(iii) a portion of an extracellular region of VEGF receptor-3 (VEGFR-3); or
(iv) any combination of (i)-(iii).

123. The AAV vector of claim 122, wherein the polypeptide comprises aflibercept.

124. The AAV vector of claim 113, wherein one or both of the first and second signal peptides comprise an IL2 signal peptide.

125. The AAV vector of any claim 113, wherein the nucleotide sequence further comprises two AAV inverted terminal repeats (ITRs), wherein the two AAV ITRs flank the coding sequences and promoter.

126. The AAV vector of claim 125, wherein the two AAV ITRs are or are derived from AAV2 ITRs.

127. A composition comprising the AAV vector of claim 113, wherein the composition is formulated for administration to the inner ear.

128. A composition comprising the AAV vector of claim 114, wherein the composition is formulated for administration to the inner ear.

129. A cell comprising the AAV vector of claim 113.

130. A cell comprising the AAV vector of claim 114.

131. A method of treating an inner ear disorder in a mammal, comprising:
administering a therapeutically effective amount of the adeno-associated virus (AAV) vector of claim 113, into an inner ear of a mammal.

132. A method of treating an inner ear disorder in a mammal, comprising:
administering a therapeutically effective amount of the adeno-associated virus (AAV) vector of claim 114, into an inner ear of a mammal.


SEQUENCE LISTING (61 sequences)

SEQ ID NO: 1 - protein, 412 aa, Homo sapiens
  source 1..412: note = Human
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 2 - DNA, 1239 bp, Homo sapiens
  source 1..1239: mol_type = unassigned DNA; note = Human
ctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 3 - protein, 389 aa, Homo sapiens
  source 1..389: note = Human
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 4 - DNA, 1170 bp, Homo sapiens
  source 1..1170: mol_type = unassigned DNA; note = Human
ctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccgtccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 5 - protein, 214 aa, synthetic construct
  REGION 1..214: note = Bevacizumab light chain variable domain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 453 aa, synthetic construct
  REGION 1..453: note = Bevacizumab heavy chain variable domain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 214 aa, synthetic construct
  REGION 1..214: note = Ranibizumab light chain variable domain
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 231 aa, synthetic construct
  REGION 1..231: note = Ranibizumab heavy chain variable domain
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 9 - protein, 22 aa, synthetic construct
  REGION 1..22: note = Signal peptide
MEFFKKTALAALVMGFSGAALA

SEQ ID NO: 10 - protein, 22 aa, synthetic construct
  REGION 1..22: note = Signal peptide
MKYLLPTAAAGLLLLAAQPAMA

SEQ ID NO: 11 - DNA, 17 bp, Homo sapiens
  source 1..17: mol_type = unassigned DNA; note = Human
aaataaaatacgaaatg

SEQ ID NO: 12 - protein, 431 aa, synthetic construct
  REGION 1..431: note = Aflibercept
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 206 aa, Homo sapiens
  source 1..206: note = Human
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 14 - protein, 186 aa, Homo sapiens
  source 1..186: note = Human
PVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 15 - protein, 116 aa, Homo sapiens
  source 1..116: note = Human
AHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRR

SEQ ID NO: 16 - protein, 117 aa, Homo sapiens
  source 1..117: note = Human
FAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRR

SEQ ID NO: 17 - protein, 687 aa, Homo sapiens
  source 1..687: note = Human
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRGEHCNKKAVFSRISKFKSTRNDCTTQSNVKH

SEQ ID NO: 18 - DNA, 2064 bp, Homo sapiens
  source 1..2064: mol_type = unassigned DNA; note = Human
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagaggtgagcactgcaacaaaaaggctgttttctctcggatctccaaatttaaaagcacaaggaatgattgtaccacacaaagtaatgtaaaacattaa

SEQ ID NO: 19 - protein, 733 aa, Homo sapiens
  source 1..733: note = Human
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPELYTSTSPSSSSSSPLSSSSSSSSSSSS

SEQ ID NO: 20 - DNA, 2202 bp, Homo sapiens
  source 1..2202: mol_type = unassigned DNA; note = Human
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtccccgagcctcagatcacttggtttaaaaacaaccacaaaatacaacaagagcctgaactgtatacatcaacgtcaccatcgtcatcgtcatcatcaccattgtcatcatcatcatcatcgtcatcatcatcatcatcatag

SEQ ID NO: 21 - protein, 541 aa, Homo sapiens
  source 1..541: note = Human
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKLPPANSSFMLPPTSFSSNYFHFLP

SEQ ID NO: 22 - DNA, 1626 bp, Homo sapiens
  source 1..1626: mol_type = unassigned DNA; note = Human
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagcttccaccagctaacagttctttcatgttgccacctacaagcttctcttccaactacttccatttccttccgtga

SEQ ID NO: 23 - protein, 732 aa, Homo sapiens
  source 1..732: note = Human
SKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLE

SEQ ID NO: 24 - protein, 737 aa, Mus sp.
  source 1..737: note = Mouse
YGSGSKLKVPELSLKGTQHVMQAGQTLFLKCRGEAAHSWSLPTTVSQEDKRLSITPPSACGRDNRQFCSTLTLDTAQANHTGLYTCRYLPTSTSKKKKAESSIYIFVSDAGSPFIEMHTDIPKLVHMTEGRQLIIPCRVTSPNVTVTLKKFPFDTLTPDGQRITWDSRRGFIIANATYKEIGLLNCEATVNGHLYQTNYLTHRQTNTILDVQIRPPSPVRLLHGQTLVLNCTATTELNTRVQMSWNYPGKATKRASIRQRIDRSHSHNNVFHSVLKINNVESRDKGLYTCRVKSGSSFQSFNTSVHVYEKGFISVKHRKQPVQETTAGRRSYRLSMKVKAFPSPEIVWLKDGSPATLKSARYLVHGYSLIIKDVTTEDAGDYTILLGIKQSRLFKNLTATLIVNVKPQIYEKSVSSLPSPPLYPLGSRQVLTCTVYGIPRPTITWLWHPCHHNHSKERYDFCTENEESFILDPSSNLGNRIESISQRMTVIEGTNKTVSTLVVADSQTPGIYSCRAFNKIGTVERNIKFYVTDVPNGFHVSLEKMPAEGEDLKLSCVVNKFLYRDITWILLRTVNNRTMHHSISKQKMATTQDYSITLNLVIKNVSLEDSGTYACRARNIYTGEDILRKTEVLVRDSEAPHLLQNLSDYEVSISGSTTLDCQARGVPAPQITWFKNNHKIQQEPGIILGPGNSTLFIERVTEEDEGVYRCRATNQKGAVESAAYLTVQGTSDKSNLE

SEQ ID NO: 25 - protein, 736 aa, Rattus sp.
  source 1..736: note = Rat
YCSGSKLKGPELSLKGTQHVMQAGQTLFLKCRGEAAHSWSLPTTVSQEDKKLSVTRSACGRNNRQFCSTLTLNMAQANHTGLYSCRYLPKSTSKEKKMESAIYIFVSDAGSPFIEMHSDIPKLVHMTEGRELIIPCRVTSPNITVTLKKFPFDALTPDGQRIAWDSRRGFIIANATYKEIGLLTCEATVNGHLYQTSYLTHRQTNTILDVQISPPSPVRFLRGQTLVLNCTVTTDLNTRVQMSWNYPGKATKRASIRQRIDQSNPHSNVFHSVLKINNVESRDKGLYTCRVKSGSSFRTFNTSVHVYEKGFISVKHRKQQVQETIAGKRSHRLSMKVKAFPSPEVVWLKDGVPATEKSARYSVHGYSLIIKDVTAEDAGDYTILLGIKQSKLFRNLTATLIVNVKPQIYEKSVSSLPSPPLYPLGSRQVLTCTVYGIPQPTIKWLWHPCHYNHSKERNDFCFGSEESFILDSSSNIGNRIEGITQRMMVIEGTNKTVSTLVVADSRTPGSYSCKAFNKIGTVERDIRFYVTDVPNGFHVSLEKIPTEGEDLKLSCVVSKFLYRDITWILLRTVNNRTMHHSISKQKMATTQDYSITLNLVIKNVSLEDSGTYACRARNIYTGEEILRKTEVLVRDLEAPLLLQNLSDHEVSISGSTTLDCQARGVPAPQITWFKNNHKIQQEPGIILGPGNSTLFIERVTEEDEGVYRCRATNQKGVVESSAYLTVQGTSDKSNLE

SEQ ID NO: 26 - protein, 745 aa, Homo sapiens
  source 1..745: note = Human
ASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLE

SEQ ID NO: 27 - protein, 743 aa, Mus sp.
  source 1..743: note = Mouse
ASVGLPGDFLHPPKLSTQKDILTILANTTLQITCRGQRDLDWLWPNAQRDSEERVLVTECGGGDSIFCKTLTIPRVVGNDTGAYKCSYRDVDIASTVYVYVRDYRSPFIASVSDQHGIVYITENKNKTVVIPCRGSISNLNVSLCARYPEKRFVPDGNRISWDSEIGFTLPSYMISYAGMVFCEAKINDETYQSIMYIVVVVGYRIYDVILSPPHEIELSAGEKLVLNCTARTELNVGLDFTWHSPPSKSHHKKIVNRDVKPFPGTVAKMFLSTLTIESVTKSDQGEYTCVASSGRMIKRNRTFVRVHTKPFIAFGSGMKSLVEATVGSQVRIPVKYLSYPAPDIKWYRNGRPIESNYTMIVGDELTIMEVTERDAGNYTVILTNPISMEKQSHMVSLVVNVPPQIGEKALISPMDSYQYGTMQTLTCTVYANPPLHHIQWYWQLEEACSYRPGQTSPYACKEWRHVEDFQGGNKIEVTKNQYALIEGKNKTVSTLVIQAANVSALYKCEAINKAGRGERVISFHVIRGPEITVQPAAQPTEQESVSLLCTADRNTFENLTWYKLGSQATSVHMGESLTPVCKNLDALWKLNGTMFSNSTNDILIVAFQNASLQDQGDYVCSAQDKKTKKRHCLVKQLIILERMAPMITGNLENQTTTIGETIEVTCPASGNPTPHITWFKDNETLVEDSGIVLRDGNRNLTIRRVRKEDGGLYTCQACNVLGCARAETLFIIEGAQEKTNLE

SEQ ID NO: 28 - protein, 741 aa, Rattus sp.
  source 1..741: note = Rat
ASVGLPGDSLHPPKLSTQKDILTILANTTLQITCRGQRDLDWLWPNTPRDSEERVLVTECGDSIFCKTLTVPRVVGNDTGAYKCFYRDTDVSSIVYVYVQDHRSPFIASVSDEHGIVYITENKNKTVVIPCRGSISNLNVSLCARYPEKRFVPDGNRISWDSEKGFTIPSYMISYAGMVFCEAKINDETYQSIMYIVLVVGYRIYDVVLSPPHEIELSAGEKLVLNCTARTELNVGLDFSWQFPSSKHQHKKIVNRDVKSLPGTVAKMFLSTLTIDSVTKSDQGEYTCTAYSGLMTKKNKTFVRVHTKPFIAFGSGMKSLVEATVGSQVRIPVKYLSYPAPDIKWYRNGRPIESNYTMIVGDELTIMEVSERDAGNYTVILTNPISMEKQSHMVSLVVNVPPQIGEKALISPMDSYQYGTMQTLTCTVYANPPLHHIQWYWQLEEACSYRPSQTNPYTCKEWRHVKDFQGGNKIEVTKNQYALIEGKNKTVSTLVIQAAYVSALYKCEAINKAGRGERVISFHVIRGPEITVQPATQPTERESMSLLCTADRNTFENLTWYKLGSQATSVHMGESLTPVCKNLDALWKLNGTVFSNSTNDILIVAFQNASLQDQGNYVCSAQDKKTKKRHCLVKQLVILERMAPMITGNLENQTTTIGETIEVVCPTSGNPTPLITWFKDNETLVEDSGIVLKDGNRNLTIRRVRKEDGGLYTCQACNVLGCARAETLFIIEGVQEKTNLE

SEQ ID NO: 29 - protein, 756 aa, Homo sapiens
  source 1..756: note = Human
YSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILV

SEQ ID NO: 30 - protein, 751 aa, Mus sp.
  source 1..751: note = Mouse
YSMTPPTLNITEDSYVIDTGDSLSISCRGQHPLEWTWPGAQEVLTTGGKDSEDTRVVHDCEGTEARPYCKVLLLAQTHANNTGSYHCYYKYIKARIEGTTAASTYVFVRDFKHPFINKPDTLLVNRKDSMWVPCLVSIPGLNITLRSQSSALHPDGQEVLWDDRRGMRVPTQLLRDALYLQCETTWGDQNFLSNLFVVHITGNELYDIQLYPKKSMELLVGEKLVLNCTVWAEFDSGVTFDWDYPGKQAERAKWVPERRSQQTHTELSSILTIHNVSQNDLGPYVCEANNGIQRFRESTEVIVHEKPFISVEWLKGPVLEATAGDELVKLPVKLAAYPPPEFQWYKDRKAVTGRHNPHALVLKEVTEASAGVYTLALWNSAAGLRQNISLELVVNVPPHIHEKEASSPSIYSRHSRQTLTCTAYGVPQPLSVQWHWRPWTPCKTFAQRSLRRRQQRDGMPQCRDWKEVTTQDAVNPIESLDSWTEFVEGKNKTVSKLVIQDANVSAMYKCVVVNKVGQDERLIYFYVTTIPDGFSIESEPSEDPLEGQSVRLSCRADNYTYEHLRWYRLNLSTLHDAQGNPLLLDCKNVHLFATPLEANLEEAEPGARHATLSLNIPRVAPEDEGDYVCEVQDRRSQDKHCHKKYLSVQALEAPRLTQNLTDLLVNVSDSLEMRCPVAGAHVPSIVWYKDERLLEKESGIDLADSNQRLSIQRVREEDAGRYLCSVCNAKGCVNSSASVAVEGSEDKGSME

SEQ ID NO: 31 - protein, 751 aa, Rattus sp.
  source 1..751: note = Rat
YSMTPPTLNITEDSYVIDTGDSLSISCRGQHPLEWTWRGAQEVLTTGGKDSEDTQVVQDCEGTEARPYCKVLSLAQTHANNTGSYYCYYKYIKARIEGTTAASTYVFVRDFEQPFINKPDTLLVNRKDSMWVPCLVSIPGLNITLRSQSSVLHPDGQEVLWDDRRGMRVPTLLLRDALYLQCETTWGDQDFLSNPFLVHITGNELYDIQLYPKKSLELLVGEKLVLNCTVWAEFDSGVTFDWDYPGKQAERAKWVPERRSQQTHTELSSILTIHNVSQHDLGPYVCEANNGIQQFRESTEVIVHEKPFISVEWLKGPVLEATAGDEMVKLPVKLAAYPPPEFQWYKDRKAVTGRHNPHALVLKEVTEASAGVYTLALWNSAAGLRQNISLELVVNVPPHIHEKEASSPSIYSRHSRQTLTCTTYGVPQPLSVQWHWRPWTPCKTFAQRSLRRRQPRDGMPQCRDWKEVTTQDAVNPIESLDTWTESVEGKNKTVSKLVIQDANVSAMYKCVVFNKVGQDERLIYFYVTTIPDGFSIESEPSEDPLEGQSVRLSCRADNYTYEHLRWYRLNLSTLHDAQGNPLLLDCKNVHLFATPLEANLEEAEPGARHATLSLNIPRVAPEDEGDYVCEVQDRRSQDKHCHKKYLSVQALEAPRLTQNLTDLLVNVRTSLEMRCPVAGAHVPSIVWYKDERLLEKESGIDLADSNQRLSIQRVREEDAGRYLCSVCNAKGCVNSSASVAVEGSEDKGSME

SEQ ID NO: 32 - protein, 220 aa, Homo sapiens
  source 1..220: note = Human
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 223 aa, Homo sapiens
  source 1..223: note = Human
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 34 - protein, 223 aa, Homo sapiens
  source 1..223: note = Human
TCPRCPAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYKTTPPMLDSDGSFFLYSKLTVDKSRWQQGNIFSCSVMHEALHNRFTQKSLSLSPGK

SEQ ID NO: 35 - DNA, 4474 bp, synthetic construct
  misc_feature 1..4474: note = pITR.CBA.Bevacizumab
  source 1..4474: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgtaccggatgcagctgctgagctgtatcgccctgtctctggccctggtcaccaattctgaggtgcagctggtggaatctggcggcggacttgttcaacctggcggctctctgagactgagctgtgccgcttctggctacaccttcaccaactacggcatgaactgggtccgacaggcccctggcaaaggccttgaatgggtcggatggatcaacacctacaccggcgagccaacatacgccgccgacttcaagcggagattcaccttcagcctggacaccagcaagagcaccgcctacctgcagatgaacagcctgagagccgaggacaccgccgtgtactactgcgccaagtatccccactactacggcagcagccactggtactttgacgtgtggggacagggcacactggtcacagtgtctagcgcctctacaaagggccccagcgttttcccactggctcctagcagcaagtctaccagcggaggaacagccgctctgggctgtctggtcaaggactactttcccgagcctgtgaccgtgtcctggaattctggcgctctgacaagcggcgtgcacacctttccagctgtgctgcaaagcagcggcctgtactctctgagcagcgtcgtgacagtgccaagcagctctctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctcgtgaagggcttctacccttccgatatcgccgtggaatgggagagcaatggccagccagagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtctctgagcctgtctcctggcaagcggaagagaagaggctctggcgaaggcagaggcagcctgcttacatgtggcgacgtggaagagaaccccggacctatgtatagaatgcagctcctgtcctgcattgccctgagcctggctctcgtgaccaacagcgacatccagatgacacagagccccagcagcctgtctgcctctgtgggagacagagtgaccatcacctgtagcgccagccaggacatctccaactacctgaactggtatcagcaaaagcccggcaaggcccctaaggtgctgatctacttcacaagcagcctgcactccggcgtgcccagcagattttctggctctggcagcggcaccgacttcaccctgaccatatctagcctgcagcctgaggacttcgccacctactactgccagcagtacagcaccgtgccttggacatttggccagggcacaaaggtggaaatcaagcggactgtggccgctcctagcgtgttcatctttccacctagcgacgagcagctgaagtctggcacagcctctgtcgtgtgcctgctgaacaacttctaccccagagaagccaaggtgcagtggaaagtggacaatgccctgcagagcggcaacagccaagagagcgtgacagagcaggactccaaggatagcacctatagcctgagcagcaccctgacactgagcaaggccgactacgagaagcacaaagtgtacgcctgcgaagtgacccaccagggcctttctagccctgtgaccaagagcttcaaccggggcgaatgttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 36 - DNA, 144 bp, synthetic construct
  misc_feature 1..144: note = ITR sequence
  source 1..144: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgt

SEQ ID NO: 37 - DNA, 1671 bp, synthetic construct
  misc_feature 1..1671: note = CBA sequence
  source 1..1671: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 38 - DNA, 39 bp, synthetic construct
  misc_feature 1..39: note = spacer
  source 1..39: mol_type = other DNA
ctcctgggcaacgtgctggttattgtgaccggtgccacc

SEQ ID NO: 39 - DNA, 60 bp, synthetic construct
  misc_feature 1..60: note = IL-2 secretion signal sequence
  source 1..60: mol_type = other DNA
atgtaccggatgcagctgctgagctgtatcgccctgtctctggccctggtcaccaattct

SEQ ID NO: 40 - DNA, 1359 bp, synthetic construct
  misc_feature 1..1359: note = Sequence encoding heavy chain of bevacizumab
  source 1..1359: mol_type = other DNA
gaggtgcagctggtggaatctggcggcggacttgttcaacctggcggctctctgagactgagctgtgccgcttctggctacaccttcaccaactacggcatgaactgggtccgacaggcccctggcaaaggccttgaatgggtcggatggatcaacacctacaccggcgagccaacatacgccgccgacttcaagcggagattcaccttcagcctggacaccagcaagagcaccgcctacctgcagatgaacagcctgagagccgaggacaccgccgtgtactactgcgccaagtatccccactactacggcagcagccactggtactttgacgtgtggggacagggcacactggtcacagtgtctagcgcctctacaaagggccccagcgttttcccactggctcctagcagcaagtctaccagcggaggaacagccgctctgggctgtctggtcaaggactactttcccgagcctgtgaccgtgtcctggaattctggcgctctgacaagcggcgtgcacacctttccagctgtgctgcaaagcagcggcctgtactctctgagcagcgtcgtgacagtgccaagcagctctctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctcgtgaagggcttctacccttccgatatcgccgtggaatgggagagcaatggccagccagagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtctctgagcctgtctcctggcaag

SEQ ID NO: 41 - DNA, 12 bp, synthetic construct
  misc_feature 1..12: note = Linker sequence
  source 1..12: mol_type = other DNA
cggaagagaaga

SEQ ID NO: 42 - DNA, 63 bp, synthetic construct
  misc_feature 1..63: note = T2A sequence
  source 1..63: mol_type = other DNA
ggctctggcgaaggcagaggcagcctgcttacatgtggcgacgtggaagagaaccccggacct

SEQ ID NO: 43 - DNA, 60 bp, synthetic construct
  misc_feature 1..60: note = IL-2 secretion signal sequence
  source 1..60: mol_type = other DNA
atgtatagaatgcagctcctgtcctgcattgccctgagcctggctctcgtgaccaacagc

SEQ ID NO: 44 - DNA, 645 bp, synthetic construct
  misc_feature 1..645: note = Sequence encoding light chain of bevacizumab
  source 1..645: mol_type = other DNA
gacatccagatgacacagagccccagcagcctgtctgcctctgtgggagacagagtgaccatcacctgtagcgccagccaggacatctccaactacctgaactggtatcagcaaaagcccggcaaggcccctaaggtgctgatctacttcacaagcagcctgcactccggcgtgcccagcagattttctggctctggcagcggcaccgacttcaccctgaccatatctagcctgcagcctgaggacttcgccacctactactgccagcagtacagcaccgtgccttggacatttggccagggcacaaaggtggaaatcaagcggactgtggccgctcctagcgtgttcatctttccacctagcgacgagcagctgaagtctggcacagcctctgtcgtgtgcctgctgaacaacttctaccccagagaagccaaggtgcagtggaaagtggacaatgccctgcagagcggcaacagccaagagagcgtgacagagcaggactccaaggatagcacctatagcctgagcagcaccctgacactgagcaaggccgactacgagaagcacaaagtgtacgcctgcgaagtgacccaccagggcctttctagccctgtgaccaagagcttcaaccggggcgaatgttaa

SEQ ID NO: 45 - DNA, 21 bp, synthetic construct
  misc_feature 1..21: note = Linker sequence
  source 1..21: mol_type = other DNA
gagctcgctgatcagcctcga

SEQ ID NO: 46 - DNA, 225 bp, synthetic construct
  misc_feature 1..225: note = Bovine growth hormone polyA tail sequence
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 47 - DNA, 34 bp, synthetic construct
  misc_feature 1..34: note = Linker sequence
  source 1..34: mol_type = other DNA
aagcttgaattcagctgacgtgcctcggaccgct

SEQ ID NO: 48 - DNA, 141 bp, synthetic construct
  misc_feature 1..141: note = ITR
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 49 - protein, 20 aa, synthetic construct
  REGION 1..20: note = IL-2 signal sequence
MYRMQLLSCIALSLALVTNS

SEQ ID NO: 50 - protein, 21 aa, synthetic construct
  REGION 1..21: note = T2A sequence
GSGEGRGSLLTCGDVEENPGP

SEQ ID NO: 51 - DNA, 3814 bp, synthetic construct
  misc_feature 1..3814: note = pITR.CBA.lucentis
  source 1..3814: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgtaccggatgcagctgctgagctgtatcgccctgtctctggccctggtcaccaattctgaggtgcagctggtggaatctggcggcggacttgttcaacctggcggctctctgagactgagctgtgccgcttctggctacgacttcacccactacggcatgaactgggtccgacaggcccctggcaaaggccttgaatgggtcggatggatcaacacctacaccggcgagccaacatacgccgccgacttcaagcggagattcaccttcagcctggacaccagcaagagcaccgcctacctgcagatgaacagcctgagagccgaggacaccgccgtgtactactgcgccaagtatccctactactacggcaccagccactggtactttgacgtgtggggacagggcacactggtcacagtgtctagcgcctctacaaagggccccagcgttttcccactggctcctagcagcaagtctaccagcggaggaacagccgctctgggctgtctggtcaaggactactttcccgagcctgtgaccgtgtcctggaattctggcgctctgacaagcggcgtgcacacctttccagctgtgctgcaaagcagcggcctgtactctctgagcagcgtcgtgacagtgccaagcagctctctgggcacccagacctacatctgcaatgtgaaccacaagcctagcaacaccaaggtggacaagaaggtggaacccaagagctgcgacaagacccacaccggcaagcggaagagaagaggctctggcgaaggcagaggcagcctgcttacatgtggcgacgtggaagagaaccccggacctatgtatagaatgcagctcctgtcctgcattgccctgagcctggctctcgtgaccaacagcgacatccagctgacacagagccccagcagcctgtctgcctctgtgggagacagagtgaccatcacctgtagcgccagccaggacatctccaactacctgaactggtatcagcaaaagcccggcaaggcccctaaggtgctgatctacttcacaagcagcctgcactccggcgtgcccagcagattttctggctctggcagcggcaccgacttcaccctgaccatatctagcctgcagcctgaggacttcgccacctactactgccagcagtacagcaccgtgccttggacatttggccagggcacaaaggtggaaatcaagcggactgtggccgctcctagcgtgttcatctttccacctagcgacgagcagctgaagtctggcacagcctctgtcgtgtgcctgctgaacaacttctaccccagagaagccaaggtgcagtggaaagtggacaatgccctgcagagcggcaacagccaagagagcgtgacagagcaggactccaaggatagcacctatagcctgagcagcaccctgacactgagcaaggccgactacgagaagcacaaagtgtacgcctgcgaagtgacccaccagggcctttctagccctgtgaccaagagcttcaaccggggcgaatgttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 52 - DNA, 699 bp, synthetic construct
  misc_feature 1..699: note = Sequence encoding ranibizumab heavy chain
  source 1..699: mol_type = other DNA
gaggtgcagctggtggaatctggcggcggacttgttcaacctggcggctctctgagactgagctgtgccgcttctggctacgacttcacccactacggcatgaactgggtccgacaggcccctggcaaaggccttgaatgggtcggatggatcaacacctacaccggcgagccaacatacgccgccgacttcaagcggagattcaccttcagcctggacaccagcaagagcaccgcctacctgcagatgaacagcctgagagccgaggacaccgccgtgtactactgcgccaagtatccctactactacggcaccagccactggtactttgacgtgtggggacagggcacactggtcacagtgtctagcgcctctacaaagggccccagcgttttcccactggctcctagcagcaagtctaccagcggaggaacagccgctctgggctgtctggtcaaggactactttcccgagcctgtgaccgtgtcctggaattctggcgctctgacaagcggcgtgcacacctttccagctgtgctgcaaagcagcggcctgtactctctgagcagcgtcgtgacagtgccaagcagctctctgggcacccagacctacatctgcaatgtgaaccacaagcctagcaacaccaaggtggacaagaaggtggaacccaagagctgcgacaagacccacaccggcaag

SEQ ID NO: 53 - DNA, 645 bp, synthetic construct
  misc_feature 1..645: note = Sequence encoding ranibizumab light chain
  source 1..645: mol_type = other DNA
gacatccagctgacacagagccccagcagcctgtctgcctctgtgggagacagagtgaccatcacctgtagcgccagccaggacatctccaactacctgaactggtatcagcaaaagcccggcaaggcccctaaggtgctgatctacttcacaagcagcctgcactccggcgtgcccagcagattttctggctctggcagcggcaccgacttcaccctgaccatatctagcctgcagcctgaggacttcgccacctactactgccagcagtacagcaccgtgccttggacatttggccagggcacaaaggtggaaatcaagcggactgtggccgctcctagcgtgttcatctttccacctagcgacgagcagctgaagtctggcacagcctctgtcgtgtgcctgctgaacaacttctaccccagagaagccaaggtgcagtggaaagtggacaatgccctgcagagcggcaacagccaagagagcgtgacagagcaggactccaaggatagcacctatagcctgagcagcaccctgacactgagcaaggccgactacgagaagcacaaagtgtacgcctgcgaagtgacccaccagggcctttctagccctgtgaccaagagcttcaaccggggcgaatgttaa

SEQ ID NO: 54 - protein, 233 aa, synthetic construct
  REGION 1..233: note = Ranibizumab Heavy Chain
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGK

SEQ ID NO: 55 - DNA, 4573 bp, synthetic construct
  misc_feature 1..4573: note = pITR.CBA.Lucentis.tGFP
  source 1..4573: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgtaccggatgcagctgctgagctgtatcgccctgtctctggccctggtcaccaattctgaggtgcagctggtggaatctggcggcggacttgttcaacctggcggctctctgagactgagctgtgccgcttctggctacgacttcacccactacggcatgaactgggtccgacaggcccctggcaaaggccttgaatgggtcggatggatcaacacctacaccggcgagccaacatacgccgccgacttcaagcggagattcaccttcagcctggacaccagcaagagcaccgcctacctgcagatgaacagcctgagagccgaggacaccgccgtgtactactgcgccaagtatccctactactacggcaccagccactggtactttgacgtgtggggacagggcacactggtcacagtgtctagcgcctctacaaagggccccagcgttttcccactggctcctagcagcaagtctaccagcggaggaacagccgctctgggctgtctggtcaaggactactttcccgagcctgtgaccgtgtcctggaattctggcgctctgacaagcggcgtgcacacctttccagctgtgctgcaaagcagcggcctgtactctctgagcagcgtcgtgacagtgccaagcagctctctgggcacccagacctacatctgcaatgtgaaccacaagcctagcaacaccaaggtggacaagaaggtggaacccaagagctgcgacaagacccacaccggcaagcggaagagaagaggctctggcgaaggcagaggcagcctgcttacatgtggcgacgtggaagagaaccccggacctatgtatagaatgcagctcctgtcctgcattgccctgagcctggctctcgtgaccaacagcgacatccagctgacacagagccccagcagcctgtctgcctctgtgggagacagagtgaccatcacctgtagcgccagccaggacatctccaactacctgaactggtatcagcaaaagcccggcaaggcccctaaggtgctgatctacttcacaagcagcctgcactccggcgtgcccagcagattttctggctctggcagcggcaccgacttcaccctgaccatatctagcctgcagcctgaggacttcgccacctactactgccagcagtacagcaccgtgccttggacatttggccagggcacaaaggtggaaatcaagcggactgtggccgctcctagcgtgttcatctttccacctagcgacgagcagctgaagtctggcacagcctctgtcgtgtgcctgctgaacaacttctaccccagagaagccaaggtgcagtggaaagtggacaatgccctgcagagcggcaacagccaagagagcgtgacagagcaggactccaaggatagcacctatagcctgagcagcaccctgacactgagcaaggccgactacgagaagcacaaagtgtacgcctgcgaagtgacccaccagggcctttctagccctgtgaccaagagcttcaaccggggcgaatgtggctccggagagggcagaggaagtctgctaacatgcggtgacgtcgaggagaatcctggcccaatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 56 - DNA, 642 bp, synthetic construct
  misc_feature 1..642: note = Sequence encoding ranibizumab light chain
  source 1..642: mol_type = other DNA
gacatccagctgacacagagccccagcagcctgtctgcctctgtgggagacagagtgaccatcacctgtagcgccagccaggacatctccaactacctgaactggtatcagcaaaagcccggcaaggcccctaaggtgctgatctacttcacaagcagcctgcactccggcgtgcccagcagattttctggctctggcagcggcaccgacttcaccctgaccatatctagcctgcagcctgaggacttcgccacctactactgccagcagtacagcaccgtgccttggacatttggccagggcacaaaggtggaaatcaagcggactgtggccgctcctagcgtgttcatctttccacctagcgacgagcagctgaagtctggcacagcctctgtcgtgtgcctgctgaacaacttctaccccagagaagccaaggtgcagtggaaagtggacaatgccctgcagagcggcaacagccaagagagcgtgacagagcaggactccaaggatagcacctatagcctgagcagcaccctgacactgagcaaggccgactacgagaagcacaaagtgtacgcctgcgaagtgacccaccagggcctttctagccctgtgaccaagagcttcaaccggggcgaatgt

SEQ ID NO: 57 - DNA, 63 bp, synthetic construct
  misc_feature 1..63: note = Linker sequence
  source 1..63: mol_type = other DNA
ggctccggagagggcagaggaagtctgctaacatgcggtgacgtcgaggagaatcctggccca

SEQ ID NO: 58 - DNA, 699 bp, synthetic construct
  misc_feature 1..699: note = Sequence encoding Turbo GFP
  source 1..699: mol_type = other DNA
atggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaataa

SEQ ID NO: 59 - protein, 232 aa, synthetic construct
  REGION 1..232: note = Turbo GFP
MESDESGLPAMEIECRITGTLNGVEFELVGGGEGTPEQGRMTNKMKSTKGALTFSPYLLSHVMGYGFYHFGTYPSGYENPFLHAINNGGYTNTRIEKYEDGGVLHVSFSYRYEAGRVIGDFKVMGTGFPEDSVIFTDKIIRSNATVEHLHPMGDNDLDGSFTRTFSLRDGGYYSSVVDSHMHFKSAIHPSILQNGGPMFAFRRVEEDHSNTELGIVEYQHAFKTPDADAGEE

SEQ ID NO: 60 - DNA, 3631 bp, synthetic construct
  misc_feature 1..3631: note = pITR.CBA.Eylea
  source 1..3631: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgtaccggatgcagctgctgagctgtatcgccctgtctctggccctggtcaccaattctagcgataccggcagacccttcgtggaaatgtacagcgagatccccgagatcatccacatgaccgagggcagagagctggtcatcccctgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagatctgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 61 - DNA, 1296 bp, synthetic construct
  misc_feature 1..1296: note = Sequence encoding aflibercept
  source 1..1296: mol_type = other DNA
agcgataccggcagacccttcgtggaaatgtacagcgagatccccgagatcatccacatgaccgagggcagagagctggtcatcccctgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagatctgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggataa